(19) United States
(12) Patent Application Publication
(10) Pub. No.: US 2022/0370396 A1
(43) Pub. Date: Nov. 24, 2022

(54) METHODS FOR MODULATING CONDITIONS OF THE EYE USING NOVEL LATANOPROST COMPOUND OPHTHALMIC COMPOSITIONS

(71) Applicant: Somerset Therapeutics LLC, Hollywood, FL (US)

(72) Inventors: Mandar V. Shah, Rockaway, NJ (US); Veerappan Subramanian, Warren, NJ (US); Ilango Subramanian, Warren, NJ (US)

(21) Appl. No.: 17/736,089

(22) Filed: May 3, 2022

Related U.S. Application Data
(60) Provisional application No. 63/183,634, filed on May 4, 2021.

(51) Int. Cl. A61K 31/222; A61P 27/06; A61K 9/00; A61K 47/44

(52) U.S. Cl. CPC A61K 31/222; A61P 27/06; A61K 9/0048; A61K 47/44

(57) ABSTRACT

The present invention relates to methods of preparing ophthalmological compositions comprising latanoprost along with or replaced by other prostaglandin analogs (e.g., latanoprost derivatives or analogs), a stabilizing amount of a suitable and stabilizing polyoxyl castor oil (e.g., a hydrogenated polyoxyl castor oil), and one or more pharmaceutically acceptable excipients, wherein the composition remains stable when stored at room temperature conditions and/or accelerated conditions. Further, the invention also provides methods of use of the ophthalmological compositions for conditions including lowering intra-ocular pressure and treatment of glaucoma.

### CROSS REFERENCE TO RELATED APPLICATIONS

This patent Application claims priority to U.S. Provisional Patent Application No. 63/183,634 filed May 4, 2021, entitled “Methods for Modulating Conditions of the Eye Using Novel Latanoprost Compound Ophthalmic Compositions”. This application claims the benefit of priority to, and incorporates by reference the entirety of, the above-referenced priority application.

### FIELD OF THE INVENTION

This invention relates to methods of making ophthalmological compositions, and methods of using such compositions to modulate conditions of the eye, such as in the treatment of glaucoma and related conditions.

### BACKGROUND OF THE INVENTION

Glaucoma is a widespread, sight-threatening disease usually associated with an elevated intraocular pressure (IOP) that, if not treated properly, potentially causes irreversible optic nerve and visual field damage. Management of the disorder requires life-long treatment, usually comprising the application of therapeutic eye drops.

A large range of eye drops, containing beta-blockers, alpha-2 agonists, carbonic anhydrase inhibitors, and prostaglandin analogs (sometimes simply called “prostaglandins”), are available for treating glaucoma, and for more than 20 years, prostaglandin analogs have been successfully used as a first-line treatment in glaucoma patients.

Developed and marketed prostaglandins used in treatment of eye conditions include travoprost (sold as, e.g., TRAVATAN Z™), tafluprost (sold as, e.g., ZIOPTAN™), and bimatoprost (sold as, e.g. LUMIGAN®). One of the prostaglandin derivatives, latanoprost, which is a selective prostaglandin F receptor (“FP receptor”; sometimes also referred to as an F-series prostanoid receptor) agonist, lowers intraocular pressure by promoting outflow of an aqueous humor. Latanoprost is chemically known as isopropyl (Z)-7[(1R,2R,3R,5S)3,5-dihydroxy-2-[(3R)-3-hydroxy-5-phenylpentyl] cyclopentyl]-5-heptanoate and has a molecular weight of 432.58. Its molecular formula is C₂₆H₄₀O₅. The chemical structure of Latanoprost is:

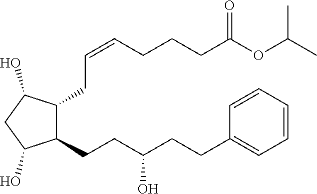 [SMILES: CC(C)OC(=O)CCC/C=C\C[C@@H]1[C@@H](CC[C@@H](O)CCc2ccccc2)[C@H](O)C[C@@H]1O]

Despite overlapping or similar functions and properties, the prostaglandins have been repeatedly demonstrated to exhibit significant differences in effects at both biological/cellular and physiological levels, such as with respect to, e.g., irritation and other adverse effects. For example, pharmacologic and pharmacokinetic data suggest the existence of a unique bimatoprost receptor, distinct from the known FP receptors; however, this receptor is yet to be cloned and bimatoprost has not been shown to work independent of FP receptor activation. See, e.g., Winkler N S et al. J Ocul Pharmacol Ther. 2014; 30(2-3):102-109. Another study reports that bimatoprost appears to reduce the IOP of patients who are unresponsive to latanoprost, suggesting that the prostamide bimatoprost and the FP receptor agonist latanoprost stimulate different receptor populations. See, e.g., Gandolfi S A, Cimino L. Ophthalmology. 2003 March; 110(3):609-14. Cytokine expression studies relating to prostaglandins have also evidenced that these compounds stimulate different cytokine pathways. For example, MMP-9 expression was higher in eyes receiving latanoprost while the MMP-2 expression was higher in eyes receiving bimatoprost with MMP1 protein levels being higher in the former. See, e.g., Reddy S et al. PLoS One. 2018 Aug. 6; 13(8). Differences in fibronectin expression and in aquaporin-1 expression in response to bimatoprost as compared to latanoprost also has been linked to variability in the IOP-lowering efficacy in some studies. See, e.g., Li X et al. PLoS One. 2016 Mar. 24. In terms of distribution, bimatoprost has been shown to reach target tissues differently than latanoprost and findings of intact bimatoprost in the target ciliary body have indicated its direct involvement in reducing IOP, and bimatoprost has been linked with poor corneal penetration when compared to latanoprost and travoprost. See, e.g., Eisenberg, D. Ophthalmology. 110(9): 1861-1862 (2003). Topical bimatoprost therapy also causes more periocular changes than latanoprost therapy. See, e.g., Karslioğlu M Z et al. Turk J Med Sci. 2015; 45(4):925-30. Prostaglandin products also are associated with differences in terms of active pharmaceutical ingredient (API) stability. For example, bimatoprost has been shown to remain stable under conditions where latanoprost and travoprost undergo degradation. See, e.g., Johnson T V et al. J Ocul Pharmacol Ther. 2011 February; 27(1):51-9.

The prostaglandins also exhibit clinical differences. For example, fewer latanoprost-treated patients reported ocular adverse events (P<0.001, latanoprost vs bimatoprost), fewer reported hyperemia (P=0.001, latanoprost vs bimatoprost), and average hyperemia scores were lower at week 12 (P=0.001, latanoprost vs bimatoprost) compared to bimatoprost-treated patients. See, e.g., Parrish R K et al. Am J Ophthalmol. 2003 May; 135(5):688-703. While latanoprost, bimatoprost, and travoprost have in some studies been shown to be comparable in their ability to reduce IOP in open angle glaucoma (OAG) and ocular hypertension (OH) patients, latanoprost exhibited greater ocular tolerability. Id. That said, nausea, vomiting, and sometimes diarrhea are considered adverse effects of travoprost and latanoprost; however, in one exemplary report, bimatoprost did not induce the same gastrointestinal adverse effects, probably due to its different chemical structure and receptors. See, e.g., Yu M et al. BMJ Case Rep. 2009; 2009:bcr08.2008.0618. Conjunctival hyperemia is more commonly associated with bimatoprost therapy, while headache was more frequent with latanoprost. See, e.g., Gandolfi S et al. Adv Ther. 2001 May-June; 18(3):110-21. However, bimatoprost provided lower mean pressures than latanoprost at every time point when studied and was statistically superior in achieving low target pressures. Id. Patients using travoprost or bimatoprost have been shown to have a significantly lower rate of adjunctive medication use compared to patients starting on latanoprost monotherapy (22.5%, 23.2%, and 30.2%, respectively), reflecting differences in overall efficacy of prostaglandins. See, e.g., Covert D et al. Curr Med Res Opin. 2006 May; 22(5):971-6.

Various formulation limitations which prostaglandin formulators face while formulating prostaglandins include poor water solubility of such active pharmaceutical ingredients; stability of such formulations over time under various conditions (including, e.g., at ambient temperature); and absorption, adsorption, or degradation of such formulations when stored in polyethylene containers. A variety of approaches have been taken in the art to attempt to address these problems. For example, these problems were considered in various prior art references which include, e.g., European Patent Publication Nos. EP 330 511 and EP 435 682, which disclose stabilization of various prostaglandins by complexing them with different cyclodextrins.

Surfactants or solubilizers have been used with other types of drugs having low water solubility. For example, U.S. Pat. Nos. 4,960,799 and 4,075,333 disclose use of non-ionic surfactants, such as polyethoxylated castor oils, as solubilizing agents in emulsion formulation/emulsion-based product formulations. Another example, U.S. Pat. No. 5,185,372, discloses topically administrable ophthalmic formulations of vitamin A which are stable preparations in which a non-ionic surfactant is used to form an emulsion of vitamin A in an aqueous medium. However, the addition of surfactants and/or solubilizers may enhance or adversely affect the chemical stability of drug compounds. See, e.g., Attwood et al. Surfactant Systems, Their Chemistry, Pharmacy, and Biology, Chapman and Hall, New York, 1983, Ch. 11, pp. 698-714. However, the efficacy of surfactants can differ significantly with respect to conditions of use, associated active pharmaceutical ingredient(s), and the amount(s) of both thereof included in the formulation.

For example, U.S. Pat. No. 5,631,287 (the “'287 patent”) describes the use of different nonionic surfactants (e.g., polysorbate 80, Alkamuls® EL-620, and Cremophor® EL) to stabilize a wide variety (more than 20 types) of prostaglandins (including a passing reference to latanoprost). Based on a few experiments performed using these surfactants and one prostaglandin (results for the other prostaglandin formulation were not shown), the '287 patent concluded that polyethoxylated castor oils enhance the chemical stability of prostaglandins, particularly as compared to polysorbate 80. A number of observations can be derived from the results of the '287 patent's experiments and associated disclosure. First, formulations with a pH of 5 performed markedly better than formulations with a pH of 7.4. Formulations with less than 0.01% prostaglandin content performed markedly (several fold) poorer than formulations with 0.01% or greater prostaglandin content. The only polyethoxylated castor oils actually tested in the '287 patent were non-hydrogenated polyethoxylated castor oils comprising 30 or 35 PEG groups, which the '287 patent indicates are preferred. These polyethoxylated castor oils performed similarly to one another in the experiments disclosed in the '287 patent. The formulations of the '287 patent include a number of consistent excipients, such as boric acid, mannitol, and disodium EDTA. The '287 patent does not discuss or investigate the effect of these excipients on prostaglandin stability. The '287 patent also does not appear to have performed any tests with formulations comprising latanoprost.

Despite the alleged enhancement in prostaglandin stability reported in the '287 patent, no latanoprost drugs incorporating such formulations were developed by either Alcon (the assignee of the '287 patent) or any other pharmaceutical company. This may be because while the stability of the undisclosed prostaglandin formulations disclosed in the '287 patent was markedly improved with the tested polyethoxylated castor oils as compared to polysorbate 80, none of the results obtained in such experiments demonstrated any kind of stability that would be suitable for pharmaceutical development (e.g., 15-25% of active pharmaceutical ingredient (“active”) was lost within 45 days in the preferred formulation at 65° C. at pH 5, a greater than 20% loss of active ingredient was shown after 50 days at 55° C. at pH 5, and greater than 50% loss (for the Alkamuis® EL-620 formulation) or 60% loss (for the Cremophor® EL formulation) of the active was reported after 50 days at 55° C. and pH 7.4. Another reason for a lack of commercial development may be because concerns about the safety of polyethoxylated castor oils were raised in the art not long after the issuance of the '287 patent, even in the context of cancer treatment (where a relatively greater number of adverse events may be acceptable compared to in the context of treating other conditions, such as most ophthalmological conditions). For example, Cremophor® EL was determined to not be “an inert vehicle, but [rather] exerts a range of biological effects, some of which have important clinical implications,” and “use [of which] has been associated with severe anaphylactoid hypersensitivity reactions, hyperlipidemia, abnormal lipoprotein patterns, [and] aggregation of erythrocytes and peripheral neuropathy.” Gelderblom H et al. Cremophor EL: the drawbacks and advantages of vehicle selection for drug formulation. Eur J Cancer. 2001 September; 37(13):1590-8. Further, Cremophor® EL's use was identified as affecting “the disposition of various drugs by changing the unbound drug concentration through micellar encapsulation”, and Cremophor® EL has been tied to toxicity, causing experts to recommend that “alternative formulations should be pursued.” Id. Regardless of the reason, the art does not appear to have attempted to further develop or improve upon the limited disclosure of the '287 patent, at least with respect to latanoprost formulations.

U.S. Pat. No. 6,235,781 (“US '781”) similarly discloses that prostaglandin derivatives are known to be unstable either because of absorption, adsorption, or degradation when stored in polyethylene containers. US '781 generally describes and claims a method of increasing the stability of an aqueous prostaglandin composition comprising a prostaglandin and a pharmaceutically acceptable surfactant wherein the method comprises: packaging the aqueous prostaglandin composition in a polypropylene container, provided that the polypropylene container is not packaged in a bag containing an iron oxide oxygen scavenger. US '781 makes passing reference to latanoprost, but only among numerous other prostaglandins, and provides no insight regarding the performance of latanoprost formulations per se. In terms of actual experimental data, the US '781 patent discloses when an aqueous composition of one or two prostaglandin derivatives (neither of which were latanoprost), including a 0.5% polyoxyl castor oil composition (Cremophor® EL), is stored in polyethylene containers, the percentage of prostaglandin derivative drug (active pharmaceutical ingredient (“API”)) remaining in the container was less than about 80% when stored at 65° C. for only four weeks, indicating unacceptable loss of the prostaglandin derivatives. Similarly, US '781 discloses that a 1.5% Cremophor® EL and EDTA formulation lost 15% of the formulation's prostaglandin derivative API when stored at 65° C. for only four weeks, an approximately 10% loss of prostaglandin derivative API in a 2% Cremophor® EL and EDTA formulation when stored at 65° C., and, finally, a nearly 40% loss of prostaglandin derivative API from a 1% Cremophor® EL and EDTA formulation when stored for four weeks at 65° C. Polypropylene containers performed markedly better, but the reported loss from, e.g., the 1% Cremophor® EL plus EDTA formulation even in polypropylene containers was about 15% of the prostaglandin API after 4 weeks of storage at 65° C. Glass containers sometimes performed similarly or better than the polypropylene containers, but generally still suffered similar losses of API in these experiments. As with the '287 patent, it does not appear that the art adopted the teaching of the '781 patent with respect to latanoprost formulations. Rather, several different solutions have since been proposed for improving latanoprost stability.

U.S. Patent Publication No. 2005/0228048 ('048) discloses latanoprost ophthalmic solution which can be stored at room temperature with stability, wherein the latanoprost is stabilized by 1) adjusting pH of the solution to 5.0 to 6.25 and/or 2) adding ε-aminocaproic acid to the solution. The '048 application makes one reference to possible inclusion of polyoxyethylene hydrogenated castor oil in connection with the various formulations disclosed therein (amongst over 30 different excipients, of various types, which apparently can be combined in an unlimited number of ways), but, of the several stabilizing agents tested did not include polyoxyethylene hydrogenated castor oil. Among the eight potential stabilizers tested in the evaluated latanoprost formulations, the '048 application found that ε-aminocaproic acid outperformed all others. Other experiments reported in the 048 application also indicated peak stability results were obtained at a pH of 5 or 5.5.

U.S. Pat. No. 8,637,054 ('054 patent) discloses preservative-free ophthalmic solutions comprising at least one prostaglandin; a solubilizing agent; a gelling agent of the carbomer type; a carbomer polymerization-inhibiting agent; and a co-gelling/co-solubilizing agent. The '054 patent includes experiments comparing a carbomer-containing latanoprost formulation to a marketed latanoprost product and reporting that the carbomer-containing formulation outperformed the on-market product in several respects unrelated to latanoprost stability. Although noting that 0.5% Cremophor® EL had been suggested as a supplement for benzalkonium chloride (“BAK” or “BKC”) in reduced BAK concentration prostaglandin formulations (citing WO 97/29752) and was incorporated with BAK into an on-market travoprost formulation, the '054 patent makes no other disclosure concerning the use of polyoxyl castor oil in regard to latanoprost formulations.

U.S. Pat. No. 9,539,262 discloses pharmaceutical compositions suitable for ophthalmic use comprising one or more prostaglandin derivatives or salts, including several references to latanoprost, in combination with a stabilizing amount of polyethylene glycol hydroxy stearate and other excipients in a pharmaceutically acceptable vehicle, sometimes including castor oil (0.15% w/v), propylene glycol, and sodium edetate. The castor oil-free formulation appears to have had better stability than the castor oil-containing formulation, and even with respect to the better stability formulation the inventors concluded that the formulation should probably be stored under refrigerated conditions or in coated containers.

As indicated, the various approaches to address the problems of latanoprost stability, such as those described in the '287 patent has not been employed in actual ophthalmological products incorporating latanoprost, whether developed at about the same time or well after the issuance of the '287 patent.

Latanoprost ophthalmic solution, 0.005%, was first approved in 1996 by the United States Food and Drug administration (US-FDA or FDA) for the reduction of elevated intraocular pressure in patients with open-angle glaucoma (OAG) or ocular hypertension (OH), and is commercially available in the United States of America, under the brand name of XALATAN® by Pharmacia & Upjohn Co. XALATAN® (or Xalatan®) is supplied as a sterile, isotonic, buffered aqueous solution of latanoprost with a pH of approximately 6.7 and an osmolality of approximately 267 mOsmol/kg. Each mL of XALATAN® contains 50 mcg of latanoprost. Benzalkonium chloride, 0.02% is included in the final formulation as a preservative. The other inactive ingredients in XALATAN® are sodium chloride, sodium dihydrogen phosphate monohydrate, disodium hydrogen phosphate anhydrous, and water for injection. One drop of XALATAN® typically contains approximately 1.5 μg of latanoprost. As used herein, reference to XALATAN® (or Xalatan®) refers to the product/composition approved by the United States Food and Drug Administration under NDA number 020597 as of Jun. 5, 1996; Jun. 5, 2006; Jun. 5, 2016; or as of May 3, 2021. Reference to XALATAN® (or Xalatan®) can be interpreted as being any one or more such compositions/products approved by US-FDA under DNA number 202597 if such products are different.

Aspects of XALATAN® and related compositions, methods, and other technical disclosures are provided in, e.g., U.S. Pat. Nos. 5,296,504, 5,422,368, and 6,429,226; US Patent Publication Number 20030149294; Alm A: Clin Ophthalmol. 2014 Sep. 26; 8:1967-85; and Patel S S and Spencer C M. Drugs Aging. 1996 November; 9(5):363-78.

In Europe, latanoprost 0.005% was also approved in 2012 as a preservative-free formulation and marketed under the brand name MONOPROST® by Laboratoires Théa, Clermont-Ferrand, France. MONOPROST® is supplied in a single-dose container as a sterile solution of latanoprost with a pH of 6.5-7.5 and an osmolality of 250-310. The MONOPROST® formulation further includes macrogolglycerol hydroxystearate 40, sorbitol, carbomer 974 P, macrogol 4000, disodium edetate, sodium hydroxide, and water for injection (“WFI”).

More recently, a benzalkonium chloride (BAK/BKC)-free 0.005% latanoprost emulsion was approved by the FDA and is marketed by Sun Pharmaceuticals under the tradename XELPROS®. The XELPROS® formulation further includes potassium sorbate 0.47%, boric acid, disodium EDTA, castor oil, sodium borate, propylene glycol, sodium hydroxide, and WFI. Aspects of XELPROS™ and related compositions, methods, and technology are described in U.S. Pat. Nos. 9,629,852 and 9,539,262.

### Construction, Definitions, and Acronyms

The following principles apply to the disclosure provided here unless contradicted by express statement, context, or plausibility.

“Uncontradicted” means not contradicted explicitly, clearly by context, or by inoperability/impossibility.

Terms e.g., “here” and “herein” means “in this disclosure.” The abbreviation “TD” similarly means “this disclosure.” Uncontradicted, any part if this disclosure is applicable to any other suitable part of TD.

The invention has several different, but related aspects. Uncontradicted, the term “aspects” refers to “aspects of this invention” (“AOTI”). The invention encompasses all aspects, as described individually, and in any suitable combination.

The primary intended audience for this disclosure (“readers”) are persons having ordinary skill in the art in the practice of the technologies discussed herein (“skilled persons”). Technological aspects of elements/steps provided here are sometimes omitted in view of the knowledge of readers. The terms “technology” and “art” here refer to the knowledge of or readily available to such skilled persons. In cases, citation of reference(s) adaptable to or otherwise related to AOTI are included here. All such patent documents and other cited publications, including those in the Background, are hereby incorporated by reference to the same extent as if each reference were individually and specifically indicated to be incorporated by reference and were set forth in its entirety herein. Uncontradicted, the content of such references (can be combinable with this disclosure e.g., components of compositions or methods therein can be combined with compositions and methods described herein). However, incorporation of patent documents is limited to the technical disclosure thereof and does not reflect on the validity, patentability, or enforceability thereof. Moreover, in the event of any conflict between TD and the teachings of such documents, the content of this disclosure will control regarding properly understanding aspects of the invention. Readers will understand that some features of cited art are not applicable to all aspects of the invention.

Heading(s) and subheadings are included for convenience. In general, heading(s) do not limit the scope of any AOTI. Uncontradicted, aspects described under one heading can apply to other aspect in TD.

The inclusion of “(s)” after an element (a step, component, feature, or the like) indicates that one or greater than one (≥1) of such an element can be present, performed, etc. E.g., “an element (or system) comprising component(s)” means an element (or system) including 1 component and an element comprising 2 or more components, each part of the statement being separate aspects and collectively representing a higher level (genus) aspect.

Uncontradicted, “a,” “an,”, “the,” and similar referents indicate both the singular and the plural form of any associated element. Uncontradicted, terms presented in the singular implicitly convey the plural and vice versa here (e.g., a passage referring to use of an “element” implicitly discloses use of corresponding “elements,” and vice versa). Uncontradicted, “also” means “also or alternatively” (abbreviated “AOA”). Terms like “combination,” “a combination,” or “and combinations,” regarding listed elements mean “a combination of any or all of such elements.” The abbreviation “CT” means “combination thereof,” with the same meaning.

The term “i.a.” (sometimes italicized) means “inter alia” or “among other things.” “AKA” means “also known as” (“also referred to as”). Uncontradicted, “elsewhere” means “elsewhere herein.”

Uncontradicted, the term “some” in respect of elements of a collection/group/class means “2 or more” of the collection/group and the term “some” regarding part of a whole means “at least 5%” (i.e., ≥5%).

Ranges here concisely refer to values within the range within an order of magnitude of the smallest endpoint. E.g., readers should interpret “1-2” as implicitly disclosing each of 1.0, 1.1, 1.2, . . . and 2.0, “5-20” as implicitly disclosing each of 5, 5.1, 5.2, . . . , 6, 6.1, 6.2, . . . 19, 19.1, . . . , 19.9, and 20, and “10-20” is to be interpreted as implicitly providing support for each of 10, 11, 12, 13, . . . , 19, and 20. Uncontradicted, ranges here include end points, regardless of how the range is described (e.g., a range “between” 1 and 5 will include 1 and 5 in addition to 2, 2.1, . . . , 3, 3.1, . . . , 4, 4.1, . . . , and 4.9), regardless of the terminology used to describe the range. Uncontradicted, applying a modifier to 1 or 2 endpoints does not change the range's value (e.g., “about 10-20” means “about 10-about 20”).

Terms of approximation, e.g., “about” or “approximately” (sometimes abbreviated as “˜”) here refer to a range of closely related values, a value that is difficult/impossible to precisely measure, or both, and, thus, include the precise value as an aspect of the disclosure (e.g., “10” is an aspect of a disclosure of “about 10” or ˜10). Similarly, readers should understand that precise values provided herein implicitly support approximately similar ranges unless contradicted. Range of approximate values depend on the value, context, and technology (e.g., criticality or operability, other evidence, statistical significance, or general understanding). In the absence of guidance here or in the art, terms of approximation (e.g., “about” or “approximately”) mean+/−10% of reference value(s).

Uncontradicted, each member of each list of elements reflects an independent aspect of the invention (often having distinct/nonobvious properties with respect to the other listed elements/aspects or features).

The conjunction “or” means “and/or” here unless contradicted (e.g., by association with a clarifying modifier e.g., “either” as in “either A or B”), regardless of any occasional use of “and/or.” A “/” symbol is sometimes used to indicate an “or” relationship between elements (e.g., “A/B” means “A or B”). Uncontradicted, a phrase e.g., “A, B, or C” or “A, B, and C” implicitly supports each of the following embodiments: A, B, and C; A, B, or C; A or C; A or B; B or C; A and C; A and B; B and C; A (alone); B (alone); and C (alone).

“Significant” and “significantly” means results/characteristics that are statistically significant using ≥1 appropriate test(s)/trial(s) in the given context (e.g., p≤0.05/0.01). “Detectable” means measurably present/different using known tools. The acronym “DOS” (or “DoS”) means “detectable(ly) or significant(ly).”

Uncontradicted, terms e.g., “including” “containing,” and “having” mean “including, but not limited to,” “including, without limitation,” or “comprising.” “Comprising” means including any detectable amount of a feature or including any detectable performance of a step. An aspect described as “comprising” or “including” a step/element can include that step/feature alone or in combination with any other associated element or step, as applicable.

Uncontradicted, terms such as “comprising” when used in reference to an element of/in a collection or composition also simultaneously provides implicit simultaneous disclosure of some of the element being present, the element making up most of the composition/collection, the element making up nearly all of the composition/collection (the composition/collection consisting essentially of the element), or the element making up all of the composition/collection or type of element in the composition/collection. Uncontradicted terms such as “comprising” and “including,” implicitly disclose corresponding aspects in which ≥1 or ≥2 of the referenced element is/are present and in which ≥1%, ≥5%, ≥10%, ≥25%, ≥33%, ≥50%, ≥65%, ≥75%, ≥90%, ≥95%, ≥99%, or 100% of the composition or collection are made up of the referenced element.

Uncontradicted, terms such as “comprising” when used in connection with a step of a method provides implicit support for (a) the method consisting essentially of the step or consisting of the step or (b) performing the step ≥2 times. Uncontradicted, terms such as “comprising” when used in connection with a step of a method and an outcome/effect provide implicit support for the step causing some, most, or all the effect (e.g., ≥1%, ≥5%, ≥10%, ≥25%, ≥33%, ≥50%, ≥65%, ≥75%, ≥90%, ≥95%, ≥99%, or 100% of the effect).

Uncontradicted, the use of “comprising,” “including,” and the like with respect to an element in a collection or composition provides implicit support for the collection/composition comprising a single instance (i.e., one of) the element, some of the element, mostly being composed of the element, generally all the collection/composition being made up of the element, and nearly all the collection/composition being made up of the element. Terms like “generally all” or “generally” means ≥70% and “nearly all” means at least 95%. “Nearly entirely” means the same thing as “nearly all.” Uncontradicted, “essentially” means “consists essentially of,” which means consisting of the referenced element/step and any other elements/steps do not materially affect the basic and novel characteristics of the applicable aspect.

Any term that provides implicit support for other, narrower aspects, should be given its broadest interpretation, unless specified.

Uncontradicted, any aspect described regarding recited element(s) also simultaneously provides implicit support for a corresponding aspect in which one, some, most, or even (if sensible) all such elements are lacking in any composition, collection, device, system, step, or method associated with the element(s). E.g., uncontradicted, where a composition is described in an aspect as comprising ingredient X, readers will understand that a corresponding aspect also implicitly provided by this disclosure lacks ingredient X, even if such an aspect is not expressly stated.

Changes to tense or presentation of terms (e.g., using “comprises predominately” in place of “predominately comprises”) do not modify the meaning of the related phrase unless indicated.

Uncontradicted, methods described here can be performed in any suitable order. Uncontradicted, devices/systems or compositions described herein can be assembled/generated in any suitable manner by any suitable method. Uncontradicted, any combination of elements, steps, components, or features of any aspects and apparent variations thereof, also are AOTI.

The percentages provided for the ingredients of the ophthalmic composition(s) of the present invention are weight/volume (w/v) percentages unless stated otherwise.

Numerous examples of aspects/elements are provided in this disclosure to illuminate the scope of the invention. The breadth of the invention or any aspect thereof should not be limited by any of the exemplary embodiments. No language in the specification should be construed as indicating any element is essential to the practice of the invention unless such a requirement is explicitly stated.

### SUMMARY OF THE INVENTION

The inventions described and claimed herein have many attributes and aspects including, but not limited to, those set forth in, e.g., described or referenced in, this Summary of the Invention (“Summary”). This Summary is not intended to be all-inclusive, and the scope of the invention is not limited to or by the aspects, features, elements, or embodiments provided in this Summary, which is included for purposes of illustration only and not restriction. Any of the aspects described under this section can be combined with any other aspect described in this section or with any other aspect of this disclosure.

In a general aspect, the invention provides a method for modulating the physiological conditions of the eye in a mammalian subject comprising administering a therapeutically effective amount of an ophthalmologically suitable composition described in this Summary or elsewhere in this disclosure. In aspects, the invention provides compositions useful in the treatment of open-angle glaucoma, elevated intraocular pressure, or both. In aspects, the invention provides a method for treating a patient diagnosed with any one or both such conditions.

In aspects, the invention provides a method for treating open-angle glaucoma or reducing intraocular pressure comprising administration of a composition as described in this Summary or otherwise in this disclosure. In one aspect, the invention provides a method of treating glaucoma, reducing intraocular pressure, or treating another condition or disease treatable by a latanoprost compound comprising administering to a subject a composition comprising latanoprost in an amount of between about 0.001% to about 0.01% by weight, polyoxyl 40 hydrogenated castor oil in an amount of between about 0.1% to about 5.0% by weight, benzalkonium chloride in an amount of between about 0.0% to about 0.02% by weight, sodium chloride in a concentration of between about 0.1% to about 0.5%, monobasic and or dibasic sodium phosphate in a concentration of between about 0.05 to about 1.0%, and water, wherein the composition remains stable when stored at room temperature conditions and/or accelerated conditions. In aspects, the invention provides methods for the treatment of glaucoma, reducing intraocular pressure, or treating another condition or disease treatable by administration of an effective amount of a latanoprost compound comprising administering an effective amount of the ophthalmologically suitable composition, usually comprising one drop of a composition, usually once per day, from a container adapted to administer eye drops to a subject a composition. In aspects, the method comprises administering the composition when the container comprising the composition can have been or has been maintained for a period of time above 15° C. prior to the administration of the composition, such as in aspects the product can or may have been stored prior to administration at a temperature of greater than about 35° C. or 40° C. for a period of more than 10 days, such as more than 2 weeks, or more than about 1 month, or the container comprising the composition can have been or has been opened and stored at temperature of greater than about 20° C. for period of about 8 weeks or longer.

In aspects, the invention provides a method of decreasing the intraocular pressure in a recipient of the method by the administration of an ophthalmologically suitable composition described herein for a period of six months by at least about 15% throughout or at the end of the treatment period.

In certain aspects, the invention provides a method for treating open-angle glaucoma or reducing intraocular pressure or both comprising administration of a composition comprising latanoprost in an amount of between about 0.001% to about 0.01% by weight, polyoxyl 40 hydrogenated castor oil in an amount of between about 0.1% to about 5.0% by weight, benzalkonium chloride in an amount of between about 0.01% to about 0.02% by weight (or up to 0.02%), sodium chloride in a concentration of between about 0.1% to about 0.5%, monobasic and or dibasic sodium phosphate in a concentration of between about 0.05% to about 1.0%, and water, wherein the composition remains stable when stored at room temperature conditions and/or accelerated conditions, and the method further comprises the administration of a second antiglaucoma agent to the subject, wherein the second antiglaucoma agent is administered separately. In aspects, the invention provides a method of administration of a combination product for the treatment of open-angle glaucoma, reducing intraocular pressure, or both, wherein the combination product is a single product, administered as a single composition, comprising an ophthalmologically suitable composition such as the composition described in this paragraph and a second antiglaucoma agent. In aspects, the invention provides a combination therapy comprising the separate administration of components described as components of a combination composition herein (e.g., comprising separate administration of a latanoprost compound composition of the invention and separately administering one or more anti-glaucoma agents or applying one or more anti-glaucoma treatments).

As noted, the aspects provided in this Summary are intended to merely exemplify the invention and summarize some of its key features, rather than limit its scope. Additional methods, related compositions, and other aspects of the invention are provided below.

### BRIEF DESCRIPTION OF THE DRAWINGS/FIGURES

FIG. 1 presents the effect of Test Item M (Xalatan®) on Dexamethasone Induced Intraocular Pressure (IOP) (normal control data provided).

FIG. 2 presents the effect of Test Item N (P0047-3-3 (1% Cremophor® RH40) formulation) on Dexamethasone-Induced Intraocular Pressure (IOP) (normal control data provided).

FIG. 3 presents the effect of Test Item M (Xalatan®) on Dexamethasone Induced Intraocular Pressure (IOP) (similar to FIG. 1, with normal control data removed).

FIG. 4 presents the effect of Test Item N (P0047-3-3 (1% Cremophor® RH40) formulation) on Dexamethasone Induced Intraocular Pressure (IOP) (similar to FIG. 2, with normal control data removed).

FIG. 5 presents the effect, as a percent change in IOP, of Test Item M (Xalatan®) and Test Item N (P0047-3-3 (1% Cremophor® RH40) formulation) on Dexamethasone Induced Intraocular Pressure (IOP).

FIG. 6 presents the 0.5 h to 24 h mean percentage change in IOP demonstrated by Test Item M (Xalatan® and Test Item N (P0047-3-3 (1% Cremophor® RH40) formulation) on Dexamethasone Induced Intraocular Pressure (IOP) Model.

FIG. 7 presents the 0.5 h to 24 h mean IOP (mmHg) demonstrated by Test Item M (Xalatan®) and Test Item N (P0047-3-3 (1% Cremophor® RH40) formulation) in the Dexamethasone Induced Intraocular Pressure (IOP) model.

FIG. 8 presents the change in IOP after a single instillation of Test Item M (Xalatan®) and Test Item N (P0047-3-3 (1% Cremophor® RH40) formulation) on Dexamethasone Induced Intraocular Pressure.

FIG. 9A provides Table 4 comprising stability data of an exemplary composition provided by the invention stored at 40°±2° C./NMT 25% RH in an ethylene oxide-sterilized container.

FIG. 9B provides Table 5 comprising stability data on the same exemplary composition as shown in FIG. 9A (Table 4) stored at 25±2° C./40±5% RH in an ethylene oxide-sterilized container.

FIG. 9C provides Table 6 comprising stability data on the same exemplary composition as shown in FIGS. 9A and 9B (Tables 4 and 5) stored at 2°-8° C. in an ethylene oxide-sterilized container.

FIG. 10A provides Table 7 comprising stability data on the same exemplary composition as shown in FIGS. 9A-9C (Tables 4-6) stored at 40°±2° C./NMT 25% RH in a gamma-sterilized container.

FIG. 10B provides Table 8 comprising stability data on the same exemplary composition as shown in FIGS. 9A-9C and FIG. 10A (Tables 4-7) stored at 25±2° C./40±5% RH in a gamma-sterilized container.

FIG. 10C provides Table 9 comprising stability data on the same exemplary composition as shown in FIGS. 9A-9C and FIGS. 10A-10B (Tables 4-8) stored at 2°-8° C. in a gamma-sterilized container.

### DETAILED DESCRIPTION OF THE INVENTION

A focused description of the various elements and characteristics of the compositions and methods of the invention is provided here. Readers will understand that although described separately, any aspects (e.g., individual embodiments) described separately/individually are intended to be capable of being combined with other one or more aspects as exemplified by other portions of the disclosure provided herein, unless contradicted by the disclosure or by impossibility. Hence, the provided disclosure of the individual aspects should be interpreted as simultaneously disclosing and describing compositions comprising any sensical combination of such aspects.

### Ophthalmologically Suitable Compositions

The present invention provides new ophthalmologically suitable compositions comprising one or more prostaglandin analogs (sometimes also called “prostaglandins” or “prostaglandin derivatives” both herein and in the art). In aspects, the one or more prostaglandin analogs in the compositions of the invention comprise latanoprost compound(s). In aspects, a latanoprost compound is the primary or only prostaglandin analog in the composition. In aspects, the primary or only ophthalmologically active compounds, or even the primary or only active pharmaceutical ingredients (“APIs”) in a composition of the invention are latanoprost compound(s). In one aspect, the invention provides ophthalmic compositions comprising one or more latanoprost compound(s) alone or along with (and in some cases replaced by) one or more other prostaglandin derivatives, wherein the composition remains stable when stored at room temperature conditions and/or accelerated conditions. In aspects, compositions provided by the invention can comprise one or more prostaglandins (e.g., prostaglandin analogues or derivatives). In aspects, compositions provided by the invention can comprise one or more latanoprost compounds. In aspects, compositions provided by the invention can comprise one or more latanoprost compounds in addition to one or more non-latanoprost compounds.

The ophthalmologically suitable compositions further comprise a polyoxyl castor oil composition component (sometimes called a “PCCC”) that detectably or significantly enhances the stability of the latanoprost compound(s) in the composition, particularly when stored at room temperature or temperatures above room temperature (e.g., under US FDA accelerated stability conditions, such as being stored at about 40° C. and about 60% relative humidity) as compared to one, some, or all on-market formulations (e.g., as compared to XALATAN® (e.g., as that product is currently marketed according to, e.g., its current US FDA approved label), other known formulations (e.g., one or more of those described in the background), or both. In aspects, the compositions of the invention provide suitable stability (e.g., retaining ≥90%, ≥93%, ≥95%, ≥96%, ≥97%, ≥98%, or ≥99% of the initial latanoprost compound content) for longer periods than achievable with a comparable amount of one, some, or all on-market products, such as XALATAN®; any, some, or all of the other compounds described in the Background; or both.

In another exemplary aspect, the invention provides ophthalmologically suitable compositions comprising about 0.001% to about 0.01% of an ophthalmologically suitable latanoprost compound composition and about 0.1% to about 5.0% of a polyoxyl castor oil composition, wherein the composition retains at least 98% of the initial latanoprost compound composition content when stored at about 22° C.-about 25° C. and about 60% relative humidity for a period of at least about 3 months. In aspects, use of the term “latanoprost compound composition” can refer, e.g., to a single latanoprost compound or, e.g., one or more latanoprost compounds as is described below. Therefore, in aspects, the invention provides ophthalmologically suitable compositions comprising about 0.001% to about 0.01% of an ophthalmologically suitable latanoprost compound or about 0.001% to about 0.01% of a mixture of ophthalmologically suitable latanoprost compounds, e.g., 2 or more latanoprost compounds, and, further, about 0.1% to about 5% of a polyoxyl castor oil composition, wherein the composition retains at least 98% of the initial latanoprost compound or mixture of latanoprost compounds (latanoprost compound composition) content when stored at about 22° C.-about 25° C. and about 60% relative humidity for a period of at least about 3 months.

In aspects, the invention provides ophthalmologically suitable compositions comprising latanoprost compound(s) and one or more polyoxyl castor oil compositions wherein the polyoxyl castor oil composition(s) at least primarily comprises a polyoxyl castor oil compound comprising between about 30 and about 60 oxyethylene units.

In aspects, the invention provides ophthalmologically suitable compositions comprising latanoprost compound(s) and polyoxyl castor oil composition(s), wherein the polyoxyl castor oil composition(s) primarily comprise(s) hydrogenated polyoxyl castor oil compound(s).

In aspects, the invention provides ophthalmologically suitable compositions as described in any of the paragraphs of this Summary having a pH of between about 6.5-about 7.

The term “ophthalmologically suitable” is generally understood in the art to mean a composition that is generally appropriate for application to the eye, the area around the eye, or both (e.g., as determined by safety testing such as through one or more well-controlled clinical studies in relevant subjects resulting in a significant determination of suitability in terms of safety, toxicity, irritability, lack of other major adverse events associated with ophthalmological products, and the like). In certain embodiments, an ophthalmologically suitable composition is a composition which does not detectably or significantly irritate or inflame the eye or the area surrounding the eye (e.g., in a significant number of patients as determined through such above-referenced studies), cause significant eye irritation, or cause the receiving subject to experience significant discomfort due to its application (again, typically as determined on a detectable or significant level through one or more well-controlled studies). Accordingly, the term “ophthalmologically suitable” is given its standard meaning when used herein. Aspects and characteristics of the ophthalmologically suitable compositions of the invention are described individually, in detail, in the following sections of this disclosure. Terms such as composition and formulation also are used herein to describe such compositions, sometimes without reference to the ophthalmologically suitable characteristics of such compositions. Nonetheless, readers will understand that aspects of this invention are generally, essentially, or only directed to ophthalmologically suitable compositions/formulations.

### Latanoprost Composition Components

“Latanoprost compounds” include not only latanoprost per se, but also any pharmaceutically/ophthalmologically acceptable derivatives of latanoprost, as well as any pharmaceutically acceptable salts, pharmaceutically acceptable solvates, pharmaceutically acceptable hydrates, pharmaceutically acceptable enantiomers, pharmaceutically acceptable derivatives, pharmaceutically acceptable polymorphs, and pharmaceutically acceptable prodrugs of any thereof. Pharmaceutical suitability in this context and generally in this disclosure is given its ordinary meaning, like ophthalmological suitability, discussed above (non-toxic, safe for use, associated with an acceptable level of adverse events, and exhibiting suitable levels of pharmaceutical activity for the intended purpose, e.g., as determined by one or more well-controlled studies at a detectable or significant level). Uncontradicted, pharmaceutical suitability is equivalent to ophthalmological suitability.

Latanoprost derivatives include any suitable type of analog or derivative. Suitability will be determined on context, but on basis of, e.g., safety, potency, efficacy, and the like. Examples of latanoprost derivatives known in the art include hydrogen sulfide-releasing compounds such as ACS67 (see, e.g., Osborne, Neville & Ji, Dan & Abdul Majid, Aman Shah & Fawcett, Rebecca & Sparatore, Anna & Soldato, Piero. (2009). ACS67, a Hydrogen Sulfide-Releasing Derivative of Latanoprost Acid, Attenuates Retinal Ischemia and Oxidative Stress to RGC-5 Cells in Culture. Investigative ophthalmology & visual science. 51. 284-94; and Perrino E, Uliva C, Lanzi C, Soldato P D, Masini E, Sparatore A. New prostaglandin derivative for glaucoma treatment. Bioorg Med Chem Lett. 2009 Mar. 15; 19(6):1639-42); nitric oxide-donating compounds, such as, e.g., compounds derived by NicOx SA and Pfizer as reported in Ophthalmology Times online on Mar. 15, 2007 by Sophia Antipolois (France)); or others similar latanoprost compounds known in the art, if suitable. Latanoprost derivatives exclude known analogs of latanoprost (i.e., other prostaglandin derivatives such as bimatoprost, travoprost, and tafluprost). Persons of ordinary skill in the art will be able to generate latanoprost derivatives and assess the suitability of known or generated latanoprost derivatives with application of routine suitability testing methods known in the art.

The amount of the latanoprost compound(s) present in the composition can be any suitable amount of any suitable number of latanoprost compound(s). In aspects, ophthalmologically suitable compositions of the invention can comprise one or more latanoprost compound(s), such as, e.g., about 1, 2, 3, 4, 5, 6, 7, 8, 9, or 10 or more latanoprost compound(s). In aspects, one of such one or more latanoprost compounds is latanoprost. In aspects herein, the phrase “latanoprost compound composition” within a composition provided by the invention can be interpreted to mean one or more latanoprost compounds, e.g., a single latanoprost compound or, e.g., ˜2, ˜3, ˜4, ˜5, ˜6, ˜7, ˜8, ˜9, or, e.g., ˜10 or more latanoprost compound(s).

In aspects, the latanoprost compound(s) of the compositions of the invention primarily comprise, ≥˜75% comprise, ≥˜90% comprise, consist essentially of, or consist of latanoprost (rather than a derivative of latanoprost).

In aspects, ophthalmologically suitable compositions of the invention can comprise one or more latanoprost compound(s) such that the total concentration of latanoprost compound(s) in the composition is between about 0.0001% and about 0.03% by weight, such as between about 0.0002% and about 0.025% by weight, between about 0.0004% and about 0.02% by weight, between about 0.0006% and about 0.015% by weight, between about 0.0008% and about 0.0125% by weight, such as between about 0.001% and about 0.01% by weight. In some respects, the ophthalmic compositions of the invention comprise between approximately 0.0025% and about 0.0075% latanoprost compound composition, such as, e.g., approximately 0.005% of latanoprost compound(s) by weight. In aspects, the total concentration of latanoprost compound in the composition is contributed by a single latanoprost compound. In aspects, ophthalmologically suitable compositions of the invention comprise between about 0.001 and about 0.01% by weight of latanoprost compound. In certain aspects, ophthalmologically suitable compositions comprise between about 0.003%-about 0.005% of latanoprost or a latanoprost compound. In aspects, the compound is latanoprost.

### Polyoxyl Castor Oil Composition Components

In aspects, compositions of the invention comprise a component characterizable as a surfactant, an emulsifier, or both. In aspects, such a component DOS enhances the stability of the active pharmaceutical ingredient(s) in the compositions herein. In certain aspects, such a component comprises, primarily comprises, generally consists of, consists essentially of, or consists of a polyoxyl castor oil composition component (a “PCCC”).

In aspects, compositions of the invention comprise a polyoxyl castor oil composition component (PCCC) that detectably or significantly enhances the stability of the latanoprost compound(s) of the composition in one or more contexts. In aspects, the PCCC acts as a surfactant. In certain aspects, the PCCC is a non-ionic surfactant. In certain aspects, the PCCC is characterizable as an emulsifier.

In aspects, the ophthalmologically suitable compositions herein can comprise polyoxyl castor oil compositions wherein the PCCCs make up at least about 60%, ≥˜65%, ≥˜70%, ≥˜75%, ≥˜80%, ≥˜85%, ≥˜90%, ≥˜95% or ≥˜99% of the non-ionic surfactant content in the composition. In aspects, the PCCC of the composition is the only detectable or only non-ionic surfactant in the composition(s).

According to aspects, compositions of the invention can comprise any suitable amount of a polyoxyl castor oil composition component (PCCC). In certain aspects, ophthalmologically suitable compositions of the invention can comprise between about 0.05% and about 10% of a PCCC, such as between ˜0.06 to ˜9%, between ˜0.07 to ˜8%, between about 0.08 to ˜7%, between ˜0.09-˜6%, or, e.g., between about 0.1% and about 5% of a PCCC (e.g., about 0.25-5%, about 0.5-5%, about 0.75-3.5%, about 0.75-3%, about 0.8-4%, about 0.8-3.2%, about 0.33-3.3%, about 0.4-4%, about 0.4-2%, about 0.5-2.5%, or about 0.75-2.25% of a PCCC). In certain aspects, ophthalmologically suitable compositions of the invention comprise between about 0.09% to about 2.1% of a PCCC, such as, e.g., about 0.65% to about 2.0% by weight of a PCCC, such as, e.g., about 0.85-1.5% of a PCCC, such as about 1.0% by weight of a PCCC. In aspects, ophthalmologically suitable compositions of the invention comprise a PCCC making up ˜0.25-2.5% of the composition, such as between about 0.3-1.5% of the composition, as in, e.g., between about 0.5-1.5% of the composition, between about 0.75-1.25% of the composition, or, e.g., about 0.9-1.1% of the composition.

In aspects, the polyoxyl castor oil composition component typically is composed of polyoxyl castor oil compound(s) in an amount suitable for imparting detectable or significant stability effects with respect to the latanoprost compound(s) in the composition. In aspects, at least about 10%, e.g., at least about 25%, at least about 33%, or at least half of the polyoxyl castor oil composition component is composed of polyoxyl castor oil compound(s). In aspects, other portions of the PCCC are made up of non-polyoxyl castor oil compound(s), as exemplified elsewhere herein.

In certain aspects, the PCCC component is composed of polyoxyl castor oil compound(s) comprising a number of polyoxyl groups. In one aspect, the PCCC component is a polyoxyl 40 hydrogenated castor oil composition. For example, in one aspect, the polyoxyl castor oil composition is Cremophor® RH40, a suitable PCCC comprising polyoxyl castor oil compound(s) according to the following chemical structure:

### General Structure of Cremophor® RH40 (Formula 1)

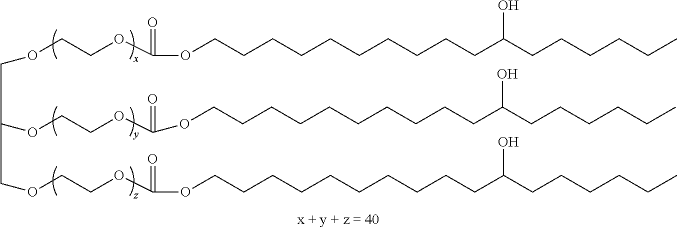

In certain aspects, suitable compounds of the PCCCs used in compositions herein can comprise compound(s) according to Formula 1, wherein x+y+z=a number other than 40, such as e.g., a number other than 40 between 20-60, 30-60, 35-60, 38-60, 38-50, 38-46, 38-44, or 38-42. Also or alternatively, suitable compounds of the PCCCs used in the compositions herein can comprise compounds wherein x+y+z=˜39, ˜38, ˜37, ˜36, ˜35, ˜34, ˜33, ˜32, ˜31, or ˜30 or even lower, or, for example, ˜41, ˜42, ˜43, ˜44, ˜45, ˜46, ˜47, ˜48, ˜49, or ˜50 or even higher, e.g., ˜52, ˜55, or ˜58.

In other aspects, polyoxyl castor oil compound(s) of a PCCC can comprise any suitable number of polyoxyl groups, regardless of whether such PCCC compounds vary from the structure of Formula 1 or not. In aspects, polyoxyl castor oil compound(s) can comprise between ˜25-70, ˜25-65, ˜25-60, ˜25-55, ˜25-50, ˜25-45, or ˜25-40, such as for example between ˜30-70, ˜35-70, ˜40-70, ˜45-70, ˜50-70, ˜55-70, ˜60-70, or ˜65-70 polyoxyl groups, as in between about 30-60, about 30-50, about 30-40, about 40-60, or between about 40-50 polyoxyl groups. In certain aspects, polyoxyl castor oil compound(s) of a PCCC can comprise at least 36 or more polyoxyl groups.

Accordingly, in aspects, one, some, most, generally all, essentially all, or all of the PCCCs in ophthalmologically suitable compositions comprise a higher degree of polymerization than Cremophor® EL (i.e., having more than 35 polyoxyl groups.)

In aspects, the ophthalmologically suitable compositions described herein can comprise a PCCC wherein at least about 50%, at least ˜55%, at least ˜60%, at least ˜65%, at least ˜70%, at least ˜75%, at least ˜80%, at least ˜85%, at least ˜90%, or at least about 95% or greater of the polyoxyl hydrogenated castor oil compound(s) in the hydrogenated castor oil composition comprise about 30-60, ˜32-60, ˜34-60, ˜36-60, ˜38-60, or, e.g., about 40-60, ˜40-58, ˜40-56, ˜40-54, ˜40-52, ˜40-50, 40-48, ˜40-46, ˜40-44, or ˜40-42 oxyethylene units, such as, e.g., ˜35 to ˜58, ˜35 to ˜56, ˜35 to ˜54, ˜35 to ˜52, or, e.g., ˜35 to ˜50 oxyethylene units. In aspects, all essentially all, e.g., all detectable or all of the polyoxyl hydrogenated castor oil compound(s) in a hydrogenated castor oil composition of a composition of the invention comprise about 40-60 oxyethylene units, such as about 35-about 50 oxyethylene units.

In aspects, polyoxyl groups described here with respect to PCCCs also or alternatively can be polyoxyl ethylene groups/units.

In some respects, the polyoxyl castor oil composition comprises polyoxyl castor oil compounds which are non-hydrogenated. In certain common aspects, the PCCC comprises hydrogenated castor oil compounds. In some respects, the PCCC primarily comprises, generally only comprises, essentially comprises, or comprises only hydrogenated castor oil compounds (with respect to PCCCs in the composition).

In aspects, the ophthalmologically suitable compositions described herein comprise a PCCC wherein at least 75% of the polyoxyl castor oil compound(s) in the polyoxyl castor oil composition are hydrogenated. In aspects, at least all detectable or all such compounds are of the PCCC are hydrogenated.

In aspects, a PCCC of the invention is a pegylated castor oil or a hydrogenated castor oil. In aspects, such an oil can comprise a single compound or molecule or also or alternatively such an oil can be a mixture of molecules.

In aspects, the ophthalmologically suitable compositions herein comprise PCCCs comprising one or more polyoxyl hydrogenated castor oil compound(s) which make up 65-85% of the polyoxyl hydrogenated castor oil composition.

In aspects, a PCCC, such as a pegylated castor oil or hydrogenated castor oil, comprises or is a mixture of hydrophobic and hydrophilic molecules. E.g., in some respects, a PCCC of the compositions described herein can be a mixture of hydrophobic and hydrophilic molecules. In aspects for example, about 70-90%, e.g., between ˜72-88%, ˜74-86%, ˜76-84% of such a PCCC is relatively hydrophobic. In aspects, about 75% of a PCCC is comprised of relatively hydrophobic molecules. In aspects, at least ˜10%, such as at least about 15% or at least about 20%, such as, e.g., between about 10-30% of a PCCC is relatively hydrophilic, such as, e.g., between ˜12-28, ˜14-26, ˜16-24, or, e.g., ˜16-22, ˜16-20, or ˜16-18% of the PCCC mixture is comprised of relatively hydrophilic components, e.g., is composed of one or more hydrophilic compounds. In aspects, about 25% of the PCCC mixture is comprised of relatively hydrophilic components.

In aspects, components of a PCCC's relatively hydrophobic portion can comprise or even be mostly comprised of any one or more hydrophobic molecules known in the art to be suitably present in types of such compositions suitable for pharmaceutical uses, including but not limited to glycerol polyethylene glycol ricinoleate, polyethylene glycol 12-oxystearate, glycerol polyethylene glycol hydroxy-stearate, or mixtures thereof. In aspects, such molecules are present together with fatty acid glycerol polyglycol esters to form a hydrophobic part of the PCCC composition.

In aspects, components of a PCCC's relatively hydrophilic portion can comprise or even be mostly comprised of any one or more hydrophilic molecules known in the art to be present in such compositions, including but not limited to polyethylene glycols, glycerol ethoxylates, and mixtures thereof. In certain embodiments, compositions herein comprise PCCCs wherein some, most, essentially all, or all, such as, e.g., at least about 10%, at least ˜20%, at least ˜30%, at least ˜40%, at least ˜50%, at least ˜60%, at least ˜70% or even more, e.g., at least ˜15-35% of the PCCC is composed of one or more hydrophilic compounds, such as, e.g., is at least primarily composed of polyethylene glycol, glycerol ethoxylates, or a mixture thereof.

In aspects, a class of castor oils particularly suitable for the compositions herein are referred to as “Cremophors” (because many such products are commercially available previously under the trademarks CREMOPHOR® or currently KOLLIPHOR® (and sold by BASF Corp.). In aspects, such compositions are synthesized by reacting either castor oil or hydrogenated castor oil with varying amounts of ethylene oxide.

In some respects, ophthalmologically suitable compositions described herein can comprise one or more latanoprost compounds, e.g., latanoprost, and a PCCC comprising hydrogenated compounds, such as, e.g., the PCCC can be or can comprise a polyoxyl hydrogenated castor oil. Polyoxyl hydrogenated castor oils suitable for the invention herein can comprise but may not be limited to, e.g., polyoxyl 35 castor oil (e.g., Cremophor® EL), polyoxyl 40 castor oil (e.g., Marlowet 40, Emulgin RO 40), a polyoxyethylene hydrogenated castor oil (such as, e.g., polyoxyethylene hydrogenated castor oil 10/polyoxyl 10 hydrogenated castor oil, polyoxyethylene hydrogenated castor oil 40/polyoxyl 40 hydrogenated castor oil (Cremophor RH 40/Kolliphor RH40), polyoxyethylene hydrogenated castor oil 50/polyoxyl 50 hydrogenated castor oil, and polyoxyethylene hydrogenated castor oil 60/polyoxyl 60 hydrogenated castor oil (Cremophor® RH 60)). Other suitable PCCCs can comprise but may not be limited to Solutol HS 15 or other similar compositions having similar or equivalent properties (e.g., having properties such as HLB, critical micelle concentration, etc.) In aspects, such compositions can further comprise one or more pharmaceutically acceptable excipients. In aspects, such compositions remain stable when stored at room temperature conditions and/or accelerated conditions.

In some aspects, the PCCC of the ophthalmologically suitable compositions of the invention can have a critical micelle concentration that lies between about 0.005 and about 0.04% (at about 37 degrees Celsius), such as, e.g., between ˜0.008-0.04% or between ˜0.009-0.04, such as between ˜0.01-0.04%, such as between ˜0.012 and ˜0.038%, between ˜0.014 and ˜0.036%, between ˜0.016 and ˜0.034%, between ˜0.018 and ˜0.032%, or, e.g., can have a critical micelle concentration of between about 0.02 and about 0.03%. In some respects, the PCCC has a critical micelle concentration of about 0.03% at 37 degrees Celsius. In certain aspects, the PCCC has a critical micelle concentration higher than 0.02%. In certain aspects, the PCCC has a critical micelle concentration higher than that of Cremophor® EL.

In aspects, the polyoxyl castor oil composition component in the composition produces emulsions with particle sizes that are detectably or significantly larger than those produced by emulsions emulsified by Cremophor® EL. In aspects, the polyoxyl castor oil composition component in the composition produces emulsions with particle sizes in a detectably or significantly larger range of particles than the range of particle sizes in an emulsion emulsified by Cremophor® EL.

In aspects, the average droplet size formed by a PCCC when in an emulsion formulation is between about 60 and about 80 nanometers (nm), such as, e.g., between ˜62-80, ˜64-80, ˜66-80, ˜68-80, or between ˜70-80 nm, such as for example between ˜60-78, ˜60-76, ˜60-74, ˜60-72, ˜60-70, or between about ˜60-68 nm. In certain aspects, the average droplet size formed by a PCCC is between ˜65 and ˜68 nm. In certain aspects, the average droplet size formed by a PCCC is less than 70 nm, such as less than about 69 nm, or less than ˜68 nm.

In certain aspects, suitable PCCCs of the invention or suitable polyoxyl castor oil compound(s) in PCCCs are characterizable by their packing parameter. Packing parameter is a characteristic defined by the formula “v/al”, wherein “v” is the hydrophobic volume, “a” is the hydrophilic area, and “l” is the hydrophobic chain length. In aspects, some, most, generally all, or all PCCCs used in compositions of the invention have a detectably or significantly smaller packing parameter than Cremophor® EL.

In aspects, the PCCC or some, most, generally all, or all polyoxyl castor oil compounds of a PCCC has a polydispersity index (PDI) of between about 0.1 and about 0.2, such as, e.g., between ˜0.1 and ˜0.19, between ˜0.1 and ˜0.18, between ˜0.1 and ˜0.17, between ˜0.1 and ˜0.16, or, e.g., between ˜0.1 and ˜0.15, such as for example between ˜0.15 and ˜0.2, or between 0.15 and ˜0.19. In aspects, a PCCC of the ophthalmologically suitable compositions of the invention or the polyoxyl castor oil compounds of the PCCC of a composition have a PDI of between about 0.15 and about 0.16. In certain aspects, some, most, generally all, essentially all, or all polyoxyl castor oil compounds of the PCCC of a composition have or the PCCC of a composition has a PDI of less than 0.17.

In aspects, the PCCC in the compositions described herein has a hydrophilic-lipophilic balance (HLB) that lies between ˜11 and ˜17, such as, e.g., between ˜12 and ˜17, such as between ˜13 and ˜17 or between ˜14 and ˜17, such as, e.g., between ˜12 and ˜16, between ˜12 and ˜15, between ˜12 and ˜14, or between ˜13 and ˜16, or, e.g., between ˜14 to ˜16.

In aspects, the average molecular weight of some, most, ≥˜75%, ≥˜90%, or ≥˜95% of the polyoxyl castor oil compound(s) in the polyoxyl castor oil component of the composition is/are greater than about 1700 g/mol-1, such as greater than about 1800 g/mol-1, greater than about 1900 g/mol-1, or greater than about 2000 g/mol-1, such as, e.g., greater than ˜2050 g/mol-1, greater than ˜2100 g/mol-1, greater than ˜2150 g/mol-1, greater than ˜2200 g/mol-1, greater than ˜2250 g/mol-1, greater than ˜2300 g/mol-1, greater than ˜2350 g/mol-1, greater than ˜2400 g/mol-1, greater than ˜2450 g/mol-1, greater than about 2500 g/mol-1, or even higher. In aspects, the average molecular weight of the polyoxyl castor oil compound(s) in the PCCC component of the ophthalmologically suitable compositions is at least about 15%, such as at least ˜16%, at least ˜17%, at least ˜18%, at least ˜19%, or at least ˜20% greater than the average molecular weight of the polyoxyl castor oil compounds of Cremophor® EL.

In certain aspects, the PCCC of the compositions herein comprise a detectably or significantly higher amount/degree of ethoxylation than that of Cremophor® EL.

In certain aspects, a PCCC of the compositions herein can DoS improve the solubility of one or more components of the compositions. In aspects, the solubility of the active pharmaceutical ingredient in the compositions of the invention, e.g., latanoprost compounds (e.g., specifically, latanoprost) is at least about 0.5%, ˜1%, ˜2%, ˜3%, ˜4%, or at least about 5% or more higher in the composition comprising the PCCC than in a composition lacking the PCCC.

In certain aspects, a PCCC of the compositions herein can DOS improve upon the permeability, bioavailability, or both, of one or more active pharmaceutical ingredients of the composition, e.g., can improve upon the permeability, bioavailability, or both of a latanoprost compound, e.g., latanoprost. In aspects, the permeability (e.g., of optical tissue) of the active pharmaceutical ingredient in the compositions of the invention, e.g., latanoprost compounds (e.g., specifically, latanoprost) is at least about 0.5%, ˜1%, ˜2%, ˜3%, ˜4%, or at least about 5% or more higher in the composition comprising the PCCC than in a composition lacking the PCCC. In aspects, the bioavailability of the active pharmaceutical ingredient in the compositions of the invention, e.g., latanoprost compounds (e.g., specifically, latanoprost) is at least about 0.5%, ˜1%, ˜2%, ˜3%, ˜4%, or at least about 5% or more higher in the composition comprising the PCCC than in a corresponding composition lacking the PCCC.

In aspects, ophthalmologically suitable compositions of the invention comprising a PCCC are capable of retaining a DoS higher amount (content) of one or more latanoprost compounds when the composition is stored at about 22° C.-about 25° C. and about 60% relative humidity for a period of at least about 3 months. This is described further elsewhere herein.

In some respects, the PCCC of a composition does not impart DOS biological effects in the recipient of the composition. According to aspects, the PCCC of a composition is inert. In aspects, the PCCC of a composition does not impart an effect having a direct clinical implication; that is, in aspects, the ophthalmologically suitable compositions described herein impart clinical effects related to the one or more latanoprost compounds whereby no additional statistically significant clinical effect is imparted by a PCCC of the composition.

In certain aspects, the PCCCs of the compositions of the invention are characterizable as non-foaming; e.g., in aspects, suitable PCCCs of the composition described herein are characterizable as showing little tendency to form a foam (no detectable foaming or significant amount of foam formation) or are otherwise known in the art not to be likely to form a foam.

In one aspect, a PCCC of the compositions herein does not DoS cause severe anaphylactoid hypersensitivity reactions, hyperlipidemia, abnormal lipoprotein patterns, aggregation of erythrocytes, peripheral neuropathy, or any combination thereof.

In one aspect, a PCCC does not cause a DOS toxic response/reaction to the composition comprising the PCCC.

In a specific aspect, a suitable PCCC of the composition is a polyoxyl 35 castor oil formulation, also known as Cremophor EL (BASF, Inc., Ludwigshafen, Germany) or Etocas 35 (Croda, Inc., Parsippany, N.J., USA). In certain alternative aspects, the PCCC of the composition is not polyoxyl 35 castor oil (e.g., is not Cremophor EL).

In another specific aspect, a suitable PCCC of the composition is an emulsifying agent obtained by reacting hydrogenated castor oil with ethylene oxide. In aspects, such a PCCC is a PEG-40 hydrogenated castor oil, e.g., Cremophor RH40 (also known as Kolliphor® RH40).

In aspects, the PCCCs suitable for the compositions herein (e.g., Cremophor® RH40) can be characterizable as soft or flowing pastes at 23 degrees Celsius (° C.). In aspects, suitable PCCCs have a very faint or no DOS odor. In aspects, suitable PCCCs can form clear solutions in water, are capable of forming clear solutions in ethanol, can form clear solutions in isopropanol, or any combination thereof. In some respects, suitable PCCCs can form clear mixtures with fatty acids. In some respects, suitable PCCCs can form clear mixtures with fatty alcohols.

In one aspect, suitable PCCCs have a sulphated ash content of ≤0.5 g/100 g, such as, e.g., ≤0.4 g/100 g or ≤0.3 g/100 g, such as, e.g., ≤0.29 g/100 g, ≤0.28 g/100 g, ≤0.27 g/100 g, ≤0.26 g/100 g, ≤0.25 g/100 g, ≤0.24 g/100 g, ≤0.23 g/100 g, ≤0.22 g/100, ≤0.21 g/100 g, ≤0.20 g/100 g, or even less.

In aspects, suitable PCCCs of the compositions herein can have an iodine value of ≤about 2.0/100 g, such as, e.g., ≤about 1.8/100 g, ≤about 1.6/100 g, ≤about 1.4/100 g, ≤about 1.2/100 g, or ≤about 1.0/100 g or even less, such as ≤about 0.8/100 g, ≤0.6/100 g, ≤about 0.4/100 g or even less.

In aspects, suitable PCCCs of the compositions herein can have a saponification value of between about 30 and about 80 mg KOH/g, such as, e.g., between ˜35-75 mg KOH/g, between ˜40-70 mg KOH/g, ˜45-65 mg KOH/g, or, e.g., between about 50-60 mg KOH/g.

In aspects, the PCCCs detectably or significantly enhance the penetration of the latanoprost compound(s) in eye tissue of subject(s).

In certain aspects, suitable PCCCs of the compositions described herein can have a hydroxyl value of between about 40 and about 95 mg KOH/g, such as, e.g., between ˜45-90 mg KOH/g, between ˜50-85 mg KOH/g, between ˜55-80 mg KOH/g, or, for example, between ˜60-75 mg KOH/g.

In certain aspects, suitable PCCCs of the compositions herein can comprise an amount of 1,4-dioxane which is less than or equal to about 20 mg/Kg, such as ≤about 18 mg/Kg, ≤about 16 mg/Kg, ≤about 14 mg/Kg, ≤about 12 mg/Kg, ≤about 10 mg/Kg, or even less, such as ≤about 8 mg/Kg, ≤about 6 mg/Kg, ≤about 4 mg/Kg, or ≤about 2 mg/Kg.

In certain aspects, suitable PCCCs of the compositions herein can comprise an acid value of less than or equal to about 2.0 mg KOH/g, such as ≤about 1.8 mg KOH/g, ≤about 1.6 mg KOH/g, ≤1.4 mg KOH/g, ≤about 1.2 mg KOH/g, ≤about 1.0 mg/KOH/g, or even less.

In certain aspects, suitable PCCCs of the compositions herein can have a pH of between about 5.5-6.6, such as, e.g., between about 5.6-6.4, ˜5.7-6.3, ˜5.8-6.2, ˜5.9-6.1, or between, e.g., ˜5-6.

In aspects, suitable PCCCs of the compositions described herein can have a water content value (as determined by a Karl Fischer titration method) of, e.g., less than or equal to about 3.0 g/100 g, such as ≤about 2.8 g/100 g, ≤about 2.6 g/100 g, ≤about 2.4 g/100 g, ≤about 2.2 g/100 g, ≤about 2.0 g/100 g, or even less, such as ≤about 1.8 g/100 g or ≤about 1.6 g/100 g or even less.

In aspects, the heavy metal content of suitable PCCCs of the invention is less than or equal to about 20 ppm, such as ≤about 18 ppm, ≤about 16 ppm, ≤about 14 ppm, ≤about 12 ppm, or ≤about 10 ppm or less, such as ≤about 8 ppm or ≤about 6 ppm or less.

### Other Excipients, Formulations, Products, and Properties Thereof

The terms “composition” and “formulation” are used interchangeably herein to describe the pharmaceutical products of the invention. The term “formulation” as used herein also or alternatively can be interchangeable with terms such as “pharmaceutical formulation”, “liquid composition”, “liquid formulation”, “composition” (e.g., the phrases, “ophthalmologically suitable composition” and “ophthalmologically suitable formulation” should be interpreted as interchangeable), referring to preparations comprising one or more latanoprost compounds in a form suitable for ophthalmic administration to a patient or subject.

The term “pharmaceutically acceptable excipient” is provided its common use definition as used herein, describing an excipient that is compatible with the other ingredients of the formulation and not deleterious to the recipient thereof, and referring to a pharmacologically inactive substance that is formulated in combination with the pharmacologically active ingredient(s) of the pharmaceutical composition. Pharmaceutically acceptable excipients can include (e.g., are inclusive of) bulking agents, fillers, diluents, products used for facilitating drug absorption or solubility, products influencing one or more pharmacokinetic parameters or considerations, and the like.

The ophthalmologically suitable compositions of the invention can be provided in any suitable form for delivery to an eye of a recipient. In aspects, the compositions herein can be provided in the form of solutions, suspensions, ointments, gels, emulsions, oils, and other dosage forms suitable for topical administration, e.g., in aspects specifically suitable for topical administration to an eye. In some respects, the compositions are in the form of an emulsion, such as an oil in water emulsion, and the compositions are provided in the form of an emulsion for topical administration. In some respects, the compositions herein are aqueous solutions. In aspects, the aqueous solutions are provided for delivery as a liquid solution.

### Aqueous Compositions

The ophthalmic formulations according to the present invention can be formulated as solutions, suspensions, ointments, gels, emulsions, oils, and other dosage forms for topical administration. In certain aspects, the ophthalmologically suitable compositions are provided as aqueous solutions. In aspects, suitable delivery forms are forms which facilitate a patient's ability to easily administer such compositions by means of instilling an appropriate dosage of composition to an eye, such as for example the application of one to two drops of the compositions in aqueous solutions in (an) affected eye(s).

In specific aspects, the ophthalmologically suitable latanoprost compound formulations of the invention are aqueous formulations. In aspects, the aqueous formulations of the invention are typically more than about 40%, ≥about 50%, ≥about 60%, ≥about 70%, ≥about 80%, or even ≥about 90% or ≥about 95% by weight water. In aspects, ≥˜75% of the ophthalmologically suitable composition is water. In aspects, some, most, generally all, or all components of the formulation are fully dissolved, such that a visibly clear, aqueous solution is provided, e.g., the aqueous solution is characterizable as a clear solution.

In some respects, the compositions of the invention are isotonic solutions. In certain aspects, the compositions of the invention are colorless/clear, aqueous, and isotonic solutions.

### Additional Excipients

The ophthalmic composition can further comprise one or more pharmaceutically acceptable excipients selected from the group consisting of solubilizing agents, thickening agents or viscosity-enhancers, chelating agents, tonicity agents, preservatives, surfactants, buffering agents, pH-adjusting agents, and a vehicle. In aspects, such a vehicle can be any suitable vehicle. In common aspects, the vehicle can be water. Exemplary tonicity agents are described herein in relation to osmolality characteristics of the compositions, and exemplary buffering agents and/or pH-adjusting agents are, at least in part, described herein in relation to the pH characteristics of the compositions.

In aspects, the solubilizing agent and the penetration enhancer can be the same or different. Exemplary solubilizer and penetration enhancer(s) can be but may not be limited to polyoxyethylene sorbitan fatty acid esters, tocopherol polyethylene glycol succinate (TPGS), poly-arginine, polyserine, tromethamine (tris), sesame seed oil or oils having similar compositions and functional characteristics suitable for ophthalmic use. Polyoxyethylene sorbitan fatty acid esters can include but may not be limited to polyoxyethylene sorbitan laurate (polysorbate 20), polyoxyethylene sorbitan palmitate (polysorbate 40), a polyoxyethylene sorbitan stearate (polysorbate 60), a polyoxyethylene sorbitan tri stearate (polysorbate 65). In some respects, polyoxyethylene sorbitan fatty acid ester ingredients can be a polyoxyethylene sorbitan oleate/polyoxyethylene sorbitan mono-oleate ester (e.g., polysorbate 80). In aspects, a solubilizer and a penetration enhancer component (which can comprise, mostly comprise, generally consist of, consist essentially of, or consist of the PCCC) can be both be present in an amount of about 0.1% to about 5.0% by weight or be present in combination in such concentration in a composition.

Thickening agents can be used in compositions to improve the form of the formulation for convenient administration and to improve contact with the eye and thereby improve bioavailability of, e.g., latanoprost. Many such ophthalmologically suitable thickening agents are known in the art. Exemplary thickening agents include, but are not limited to, polymers containing hydrophilic groups such as monosaccharides and polysaccharides, ethylene oxide groups, hydroxyl groups, carboxylic acids, or other charged functional groups. While not intending to limit the scope of the invention, some examples of useful viscosity-enhancing agents are sodium carboxymethylcellulose, hydroxypropyl methylcellulose, povidone, polyvinyl alcohol, and polyethylene glycol. In certain aspects, formulations described herein lack any thickening (e.g., viscosity-enhancing) compounds or agents.

Chelating agents can be used in the ophthalmic compositions to enhance preservative effectiveness, e.g., by forming stable water-soluble complexes (chelates) with alkaline earth and heavy metal ions. In some respects, formulations described herein can comprise one or more chelating agents. In aspects, such chelating agents can be any ophthalmologically suitable and pharmaceutically acceptable chelating agent, such as but which may not be limited to compounds capable of sequestering divalent or polyvalent metal cations and further remains effective at a pH of between about 6.5 and 8.0. In aspects, the chelating agent does not detectably or significantly impact the efficacy of any other component of the formulation. In aspects, exemplary chelating agents present in a formulation described herein can comprise but may not be limited to cromolyn, monomeric polyacids such as EDTA, cyclohexanediamine tetraacetic acid (CDTA), hydroxyethyl ethylenediamine triacetic acid (HEDTA), diethylenetriamine pentaacetic acid (DTPA), dimercaptopropane sulfonic acid (DMPS), dimercaptosuccmic acid (DMSA), aminotrimethylene phosphonic acid (ATPA), citric acid, any ophthalmologically acceptable salts thereof, and/or combinations of any two or more such compounds. In other aspects, a chelating agent can be a phosphate, such as, e.g., pyrophosphates, tripolyphosphates, and, hexametaphosphates; a chelating antibiotic such as chloroquine and tetracycline; a nitrogen-containing chelating agent containing two or more chelating nitrogen atoms within an imino group or in an aromatic ring (e.g., diimines, 2,2′-bipyridines, etc.); or for example a polyamine such as cyclam (1,4,7,11-tetraazacyclotetradecane), N—(C₁-C₃₀ alkyl)-substituted cyclams (e.g., hexadecyclam, tetramethyl hexadecyl cyclam), diethylenetriamine (DETA), spermine, diethylnorspermine (DENSPM), diethylhomospermine (DEHOP), and deferoxamine (N′-[5-[[4-[[5-(acetylhydroxyamino) pentyl] amino]-1,4-dioxobutyl]hydroxy-amino]pentyl]-N′-(5-aminopentyl)-N-hydroxybutanediamide; also known as desferrioxamine B and DFO). In certain aspects, the formulations described herein comprise EDTA or an ophthalmologically suitable EDTA salt such as, e.g., diammonium EDTA, disodium EDTA, dipotassium EDTA, triammonium EDTA, trisodium EDTA, tripotassium EDTA, or calcium disodium EDTA.

In aspects, compositions herein comprise latanoprost, a PCCC, and wherein the remainder of the composition is essentially composed of a formulation that comprises sodium chloride in a concentration of about 0.1 to about 0.5%%, monobasic or dibasic sodium phosphate in a concentration of between about 0.05 to 1%, and water.

### Preservatives, e.g., Benzalkonium Chloride

The ophthalmic formulations described herein can comprise, in aspects, one or more preservatives. In aspects, such one or more preservatives can be present in an anti-microbially effective amount which in detectably or significantly inhibits microbial growth. Antimicrobial effective amounts of a preservative may be determined by performing preservative efficacy tests or antimicrobial effectiveness tests. These tests are inter alia described in chapter 51 of the United States Pharmacopoeia 29-National Formulary 24 (USP 29-NF 24). The preservatives may be used in an amount within the concentration ranges described in standard reference books like Remington's Pharmaceutical Sciences and/or Handbook of Pharmaceutical Excipients.

In aspects, any ophthalmologically suitable preservative can be used in formulations of the invention. In aspects, exemplary preservatives can comprise but may not be limited to hydrogen peroxide; sorbic acid; biguanides; quaternary ammonium salts such as benzalkonium chloride and benzethonium chloride; cationic compounds such as chlorhexidine gluconate; p-hydroxybenzoates such as methyl p-hydroxybenzoate, ethyl p-hydroxybenzoate, propyl p-hydroxybenzoate and butyl p-hydroxybenzoate; alcohol compounds such as chlorobutanol and benzyl alcohol; sodium dehydroacetate; thiomersal and the like which may be known by those skilled in the art. In aspects, one or more preservatives may provide one or more other functional activities, such as for example detectably or significantly enhancing latanoprost penetration in the eye of subject(s).

According to certain aspects, a preservative, if present, suitable for the compositions described herein, is benzalkonium chloride (BKC). The benzalkonium chloride (abbreviated herein as BKC, though in other literature other abbreviations such as BAC, BAK, or BZK may be used), can be present to serve as a penetration enhancer, preservative, solubilizer, or any combination thereof in the formulations of the present invention. Benzalkonium chlorides, also known as alkyl dimethyl benzyl ammonium chlorides (or ADBAC), alkyl dimethyl (phenylmethyl) chlorides, or ammonium alkyl dimethyl benzyl chlorides, are a class of quaternary ammonium compounds. In aspects, benzalkonium chloride can be present in the formulation in a concentration of between about 0.0005% to 0.02%, such as between about 0.001% to about 0.02%, such as between about 0.0025% to about 0.02%, such as between about 0.00375% to about 0.02%, such as between about 0.01% to about 0.02%, or for example 0.02% by weight. In other aspects, benzalkonium chloride can be present in the formulation in a concentration greater than about 0.02%, such as about 0.03%, such as about 0.04%, such as about 0.05%, and greater. In aspects, compositions herein can comprise a preservative-enhancing or preservative-effective amount of benzalkonium chloride in a concentration of less than 0.2 mg/mL.

In certain alternative embodiments, the compositions herein are free of benzalkonium chloride. In certain aspects, compositions here are free of any excipients that typically would be primarily characterized as being preservatives.

In compositions wherein benzalkonium chloride is present in the ophthalmologically suitable compositions of the invention, BKC can be used as preservative, penetration enhancer, solubilizer, or any combination thereof, and in aspects can be present in an amount of about 0.001% to about 0.03% by weight, e.g., 0.001% to about 0.02%, such as in an amount of about 0.002%, ˜0.004%, ˜0.006%, ˜0.008%, ˜0.01%, ˜0.012%, ˜0.014%, ˜0.016%, ˜0.018%, or about 0.02%, such as ˜0.022%, ˜0.024˜, 0.026%, 0.028%, or 0.03%. In aspects, the amount of BKC in a composition, if not entirely absent, is between about 0.0025 to 0.005%% by weight.

### Exclusions

In aspects, compositions of the invention are characterizable as lacking certain components.

According to aspects, the compositions herein lack any polymer having more than about 90 monomer units, such as more than ˜95 or more than ˜100 monomer units, such as more than ˜125, more than ˜150, more than ˜175, more than ˜200, more than ˜225, or more than ˜250 monomer units. Also, or alternatively, in aspects, no compound in the composition comprises any carbon or carbon-containing chain that comprises more than about 40, more than ˜42, more than ˜44, more than ˜46, more than ˜48, or more than ˜50, such as more than ˜52, more than ˜54, more than ˜56, more than ˜58, or more than ˜60 monomer units.

In one aspect, ophthalmologically suitable compositions of the invention lack any high molecular weight polymers, e.g., homopolymers, made from acrylic acid, e.g., carbomers (often noted in the art as Carbopol® polymers). In aspects such polymers are high molecular weight polymers of acrylic acid crosslinked with allyl ethers of polyalcohols. For example, in aspects, compositions herein do not comprise, e.g., Carbopol® 934 NF, Carbopol P NF®, Carbopol 71 G NF, or the like. In aspects, compositions herein lack crosslinked polymers such as Pemulen® and Noveon® crosslinked polymers. In aspects, the compositions herein are free of any cross-linked polymers. In aspects, compositions of the invention comprise less than about 0.5%, less than about 0.4%, less than about 0.3%, less than about 0.2%, or less than about 0.1% of any polyacrylic acid polymer. In aspects, compositions are free of any detectable amount of any polyacrylic acid polymer, such as any carbomer-type polymer, e.g., carbomer 934, carbomer 934 P, carbomer 941, Carbopol 910, and carboxyvinyl polymer.

In aspects, the compositions herein lack any polymeric component having a maximum viscosity greater than about 1000 cPs, greater than about 2000 cPs, greater than about 4000 cPs, greater than about 5000 cPs, greater than about 7500 cPs, or greater than about 10000 cPs (such viscosity being determined under the applicable test conditions, such as conditions of typical use, e.g., about room temperature and normal humidity). Methods for performing viscosity testing to make such evaluations are well known in the art.

In aspects, the compositions herein lack any polymer having a molecular weight greater than about, e.g., 300,000 Da, such as greater than about 350,000 Da, 400,000 Da, 450,000 Da, or 500,000 Da as measured by gel permeation chromatography using linear polyacrylic acid as a reference.

In aspects, the compositions herein do not comprise any polymers that form or include a detectable or significant number of chemical cross-links, physical entanglements, crystalline regions, or polymer complexes like crosslinking. In aspects, compositions of the invention, if molecular weight between cross-links is present, such molecular weight between cross-links (Mc) is significantly lower than that of carbomers, e.g., is less than for example, about 300,000 g/mol, less than ˜250,000 g/mol, less than ˜200,000 g/mol, less than ˜150,000 g/mol, less than ˜100,000 g/mol, less than ˜50,000 g/mol, less than ˜25,000 g/mol, or even less. In aspects such an Mc is effectively absent.

In certain aspects, compositions described herein do not comprise a polymer characterizable as, or known in the art to be, a swellable polymer. In aspects, carbomers are known to swell in water up to for example 1000 times their original volume; in aspects, such polymers are absent from the compositions herein, wherein no polymer component swells more than about 1%, ˜2%, ˜3%, ˜4%, ˜5%, ˜6%, ˜7%, ˜8%, ˜9%, or to ˜10% of its original size.

In aspects, compositions described herein do not comprise a polymer characterizable as, or known in the art to be, gel-forming.

In aspects, compositions herein are essentially free of alcohol(s) (e.g., except for any of the alcohols described herein, free of polyvinyl alcohol, or free of any alcohol not explicitly recited), castor oil, potassium sorbate, travoprost, boric acid, aminocaproic acid, EDTA and related compositions (such as disodium EDTA), timolol, tyloxapol, trometamol, bimatoprost, sodium acetate, diclofenac, flurbiprofen, ketorolac, macrogolglycerol compounds (e.g., macrogolglycerol hydroxystearate, such as macrogolglycerol hydroxystearate 40), sorbitol, mannitol, macrogol compounds (e.g., macrogol 4000), disodium edetate, sodium hydroxide, nepafenac, sodium dihydrogen phosphate monohydrate, disodium hydrogen phosphate anhydrous, propylene glycol, or a combination of any, several, or all thereof. In aspects, compositions of the invention comprise ≤5%, ≤2%, ≤1%, ≤0.05%, ≤0.02%, ≤0.01%, ≤0.005%, or ≤0.001% of any one, some, or all of such compositions.

In aspects, compositions herein are free of castor oil, potassium sorbate, boric acid, disodium EDTA (ethylenediaminetetraacetic acid), tyloxapol, trometamol, macrogolglycerol compounds (e.g., macrogolglycerol hydroxystearate, such as macrogolglycerol hydroxystearate 40), sorbitol, macrogol compounds (e.g., macrogol 4000), disodium edetate, mannitol, sodium hydroxide, nepafenac, sodium dihydrogen phosphate monohydrate, disodium hydrogen phosphate anhydrous, propylene glycol, or a combination of any, several, or all thereof. In aspects, compositions of the invention comprise ≤5%, ≤2%, ≤1%, ≤0.05%, ≤0.02%, ≤0.01%, ≤0.005%, or ≤0.001% of any one, some, or all of such compositions.

In one aspect, compositions of the invention do not comprise Cremophor® EL.

In one aspect, compositions of the invention are preservative-free. In one aspect, compositions of the invention are free of benzalkonium chloride.

### pH and Osmolality

According to some aspects, the ophthalmologically suitable compositions of the invention can have any pH suitable for application of the compositions to an eye. In aspects, compositions are formulated to minimize or avoid injury to or irritation of the eye. In aspects, compositions are formulated to minimize or avoid the production of additional tears upon application of the compositions, to minimize or avoid flushing of the composition from the eye. According to some aspects, the ophthalmologically suitable compositions of the invention have a pH of between about 4.3 to about 8.4, such as for example between ˜4.5-8.3, ˜4.7-8.2, ˜4.8-8.1, or, e.g., between about 5.0 to about 8.0. In certain aspects, the pH of the compositions of the invention is higher than about 5.0, higher than ˜5.1, higher than ˜5.2, higher than ˜5.3, higher than ˜5.4, or, e.g., higher than ˜5.5. In aspects, the pH of the compositions of the invention is between about 6.4-7 or about 6.4-7.1, such as between about 6.5-6.9, about 6.4-7, about 6.5-6.8, about 6.4-6.8, or between about 6.4-6.8. In aspects, compositions described herein are buffered to maintain the pH within a desired range.

The ophthalmic formulations described herein can comprise one or more buffers or pH-adjusting agents. In aspects, such agents can be used to adjust the pH to a desirable range, such as between about 5 to about 8 (or, e.g., between ˜6.4 to ˜7.4, or for example ˜6.5 to ˜7.2, such as ˜6.7. In facets, any ophthalmologically suitable and pharmaceutically acceptable buffer can be used. In aspects, such buffers can include but may not be limited to monobasic and/or dibasic sodium phosphate. In aspects sodium hydroxide and hydrochloric acid can be used during the production of the formulations herein to adjust pH to a target value, e.g., to a target value of approximately about 6.5 to about 7.2, such as ˜6.7. In aspects, other buffers and other pH adjusters which are ophthalmologically suitable, pharmaceutically acceptable, and commonly used and known within the art can be present in the formulations herein. In specific aspects, the formulations described herein comprise monobasic and/or dibasic sodium phosphate. In aspects, monobasic and/or dibasic sodium phosphate is present in the formulation in a concentration of between about 0.02-about 1.5%, such as, e.g., between ˜0.03-1.4%, ˜0.04-1.2%, or, e.g., between about 0.05 to 1.0% by weight.

In aspects, presence of a buffer may contribute the tonicity of the formulation, hence in some embodiments one or more buffers and one or more tonicity agents can combine to contribute to the osmolality and osmolarity of a formulation. In aspects, the ophthalmologically suitable compositions of the invention have any osmolality or osmolarity suitable for ophthalmic applications, e.g., for providing efficacy and facilitating patient comfort when using a product comprising the compositions described herein. In aspects, compositions of the invention can have an osmolarity which can be between about 150-1960 mOsm/L, such as, e.g., between about 175 and about 1800 mOsm/L, such as between about 200 and about 1600 mOsm/L, between about 210-about 1400 mOsm/L, between about 220-about 1200 mOsm/L, between about 230-about 1000 mOsm/L, between about 240-about 800 mOsm/L, between about 250-about 600 mOsm/L, or, e.g., between about 260-about 400 mOsm/L. In aspects, compositions herein can have an osmolality of between about 250 to about 400 milliosmoles per kilogram (mOsm/Kg), such as, for example, between about 255-395 mOsm/Kg, ˜260-390 mOsm/Kg, ˜265-385 mOsm/Kg, ˜270-380 mOsm/Kg, ˜275-375 mOsm/Kg, or, e.g., between ˜280-370 mOsm/Kg.

In aspects, it is desirable that the formulations described herein be close to isotonic, e.g., having the same osmotic pressure as the membranes within the eye, and thus causing no DOS contraction or swelling of the eye tissues when administered. In aspects, one or more tonicity agents may be included in the formulation to adjust the formulation such that it is within such a desired, suitable, or optimal isotonic range. In aspects, any ophthalmologically suitable and pharmaceutically acceptable tonicity agent can be used. In aspects, exemplary tonicity agents include, but are not limited to, sodium chloride, glycerin, mannitol, sorbitol, and other electrolytes. In certain aspects, one or more tonicity agents can be present in the formulation in an amount of about 0.005% to about 0.85%, such as about 0.006% to 0.84%, between about 0.007% to about 0.83%, between about 0.008% to about 0.82%, between about 0.009% to about 0.81%, or for example between about 0.01% to about 0.8% by weight. In some respects, sodium chloride is present in the formulations described herein. In aspects, the sodium chloride is present in an amount of between about 0.1% to about 0.5% of the formulation. In aspects, sodium chloride is present in an amount of between about 0.2%-about 0.5%, between ˜0.3-0.5%, or, e.g., in an amount of about 0.4%.

### Viscosity

In aspects, the compositions herein have a readable viscosity of about 0.5-2, such as, e.g., between about ˜0.6-2, ˜0.8-2, ˜0.9-2, ˜1-2, ˜1.1-2, ˜1.2-2, ˜1.3-2, ˜1.4-2, or ˜1.5-2, such as, e.g., ˜0.5-1.9, ˜0.5-1.8, ˜0.5-1.6, ˜0.5-1.5, ˜0.5-1.4, ˜0.5-1.3, ˜0.5-1.2, ˜0.5-1.1, ˜0.5-1, such as, e.g., between about 0.7-1.7, ˜0.8-1.6, or between about 1 to about 1.5 (using the method described in Kolo and Jensen, 2017). In aspects, the compositions herein have a viscosity in centipoise (cps) between about 1 and 25 cps, such as between about 5-25 cps, between about 10-25 cps, between about 15-25 cps, between about 20-25 cps, such as, e.g., between about 1-about 20 cps, between about 1-15 cps, between about 1-10 cps, or between about 1-5 cps, such as, e.g., between about 1-15 cps or between about 10-25 cps.

### Impurities

As used herein, the term “impurity” refers to an undesired substance in a composition which may be present in an initial composition and/or may be formed after a certain period of shelf life of a composition. In some cases, such impurities may be formed via degradation of one or more components of the composition. Sources of degradation can include, but are not limited to, oxidation, light, ultraviolet light, moisture, heat, changes in pH, and composition component interactions. Impurities can arise from one or more potential sources, including, for example, the drug substance, excipients, water, the manufacturing process and equipment, and the container closure system.

In aspects, the ophthalmologically suitable compositions described herein contain less than about 0.5%, less than about 0.4%, less than about 0.3%, <˜0.29%, <˜0.28%, <˜0.27%, <˜0.26%, or, e.g., less than about 0.25% or even less of total impurities, such as, e.g., <˜0.2%, <˜0.175%, <˜0.15%, <˜0.125%, or, e.g., less than about <˜0.1% of total impurities.

### Packaging

According to certain embodiments, ophthalmologically suitable compositions of the invention can be packaged in any suitable packaging, such suitability being at least in part defined by protecting the compositions held therein from degradation, contamination, or both. In certain aspects, suitable packaging materials are materials which exhibit less than about 20%, such as <˜18%, <˜16%, <˜14%, <˜12%, <˜10%, <˜8%, <˜6%, <˜4%, <˜2% or even less sorption of a prostaglandin, e.g., of a latanoprost compound (e.g., specifically latanoprost). In some respects, suitable materials include but may not be limited to packaging material made of select polyolefins, such as, e.g., DuPont® 20 LDPE, Chevron 5502 HDPE, Atofina 3020 PP, polypropylene homopolymers, low ethylene content (≤8%) polypropylenes, and polymers (HDPE, PP) with low content of additives (<5%) and with low flexural modulus (<200 kpsi). In some respects, a suitable material is an EP-quality LDPE which, in further aspects, may contain no additives. In aspects, suitable packaging can comprise a polypropylene container provided that that polypropylene container is not packaged in a bag/container containing an iron oxide oxygen scavenger.

In certain aspects, the packaging can comprise or can be mostly comprised of (e.g., comprise in an amount ≥˜10%, ≥˜20%, ≥˜30%, ≥˜40%, or ≥˜50%, such as, e.g., comprise in an amount ≥˜60%, ≥˜70%, ≥˜80%, ≥˜90% or more) an ultraviolet-light blocking agent or material. In aspects, such a material can be capable of blocking ≥˜1%, ≥˜5%, ≥˜10%, ≥˜20%, ≥˜30%, ≥˜40%, or ≥˜50%, such as, e.g., ≥˜60%, ≥˜70%, ≥˜80%, ≥˜90% or more of the ultraviolet light in the environment from entering the container. In aspects, compositions described herein can be packaged in, stored, in, or both packaged and stored in a container wherein the container significantly reduces exposure of the latanoprost compound to UV B radiation, such as by at least about 50%, at least about 65%, at least about 75%, at least about 90%, at least about 95%, or at least 99%. In some aspect the packaging material of a composition described herein is semi- or completely opaque, while in alternative aspects, the packaging is semi- or completely clear. In aspects, packaging can comprise different parts wherein one component of the packaging comprises a first material and one or more components of the packing contain a second (or more) material(s).

In certain aspects, packaging can be selected based on the method of delivery of the compositions herein (e.g., compositions provided as a cream can be provided in suitable packaging for creams wherein compositions provided as a liquid can be provided in suitable packaging for liquids, e.g., in a user-friendly dropper bottle.) In aspects, the compositions of the invention are stored in a packaging that facilitates the delivery of the composition as eye drops. In one aspect, the ophthalmic compositions comprise latanoprost, polyoxyl 40 hydrogenated castor oil, and one or more pharmaceutically acceptable excipient(s), and are provided in single-dose bottles. In an alternative aspect, such compositions are provided in multi-dose bottles, such as multi-dose eye dropper bottles. In aspects, such multi-dose bottles allow for the composition, e.g., provided as a solution to be dropped into the recipient's eye(s), to be applied as liquid drops over several days.

In aspects, the average force required to release one or more drops of the compositions described herein from a dropper bottle (a standard bottle common in the art for dispensing liquid in droplet form), by compressing the middle section of the storage body of such a dropper bottle, ranges between about 1.7-2.8 kg for release of the first drop, e.g., between about 1.7-2.6, ˜1.7-2.4, ˜1.7-2.2, or between about ˜1.7-2.0. In aspects, successive drops can require more tension, such as can require an additional ˜20-30% of force for release of the second drop, and, e.g., an additional force of ˜24-50% for release of the third drop.

In aspects, the compositions herein are provided without a preservative. In certain aspects, the compositions described herein can maintain stability when stored at room temperature conditions and/or accelerated conditions. In certain aspects, specific packaging may be desirable or necessary depending on whether or not the formulation comprises a preservative. E.g., in aspects the container for the composition does not significantly interact with (or is considered inert with respect to) one, some, most, generally all, essentially all, or all ingredients of the composition (e.g., does not interact with BAK/BKC, which is known in the art to interact with some types of container materials).

According to one aspect, compositions herein can comprise latanoprost, polyoxyl 40 hydrogenated castor oil, and one or more pharmaceutically acceptable excipients, wherein the composition is packaged using an aseptic packaging technique such as “Blow-Fill-Seal” (single-dose unit), wherein the composition remains stable when stored at room temperature conditions and/or accelerated conditions.

Compositions of the invention also can comprise latanoprost and one or more of the listed excipients described in the Examples, can consist essentially of latanoprost and one or more of the listed excipients described in the Examples, or can consist of latanoprost and one or more of the listed excipients described in the Examples (optionally in about the amounts shown in the Examples).

### Combination Products

In aspects, the ophthalmologically suitable composition can comprise, in addition to a latanoprost compound, one or more additional active pharmaceutical ingredients (APIs), which may comprise prostaglandins or prostaglandin analogs, derivatives of any such API, their pharmaceutically acceptable salts, or the like, wherein the composition comprises an ophthalmologically effective amount of the two or more active pharmaceutical ingredient(s) (“APIs”). In aspects, the effective amount can be an effective amount of any one API alone or the combination of the two or more APIs can provide the effective number of API(s). In aspects, the second or more APIs can be selected from betaxolol, carteolol, levobunolol, timolol, brinzolamide, dorzolamide, echothiophate iodide, pilocarpine, latanoprostene bunod, tafluprost, travoprost, bimatoprost, brimonidine, netarsudil, apraclonidine, alternative forms thereof, derivatives thereof, or combinations of any two or more thereof.

In one aspect, the composition is administered in combination with, or comprises, an ophthalmologically effective amount of a betaxolol compound, such as betaxolol hydrochloride. In aspects, the betaxolol composition is provided in a separate solution or suspension or is combined in the composition with the latanoprost compound(s). In aspects, the betaxolol composition comprises a concentration of about 0.1-about 0.9%, e.g., between about 0.2-0.8%, between about 0.4-0.6%, or, e.g., such as about 0.5% of a betaxolol compound. In aspects, the betaxolol composition is administered 1-3 times per day, such as 2 times per day, through administration of 1-2 drops, e.g., about 1 drop, per affected. In aspects, about 1 drop is administered to each affected eye about two times per day.

In another aspect, the composition is administered in combination with, or comprises, an ophthalmologically effective amount of a carteolol compound, such as carteolol hydrochloride. In aspects, the carteolol composition is provided in a separate solution or suspension or is combined in the composition with the latanoprost compound(s). In aspects, the carteolol composition comprises a concentration of about 0.02 to about 2%, e.g., between about 0.4-1.8%, between about 0.6-1.6%, between about 0.8-1.4%, or, e.g., such as about 1% of a carteolol compound. In aspects, the carteolol composition is administered 1-3 times per day, such as 2 times per day, through administration of 1-2 drops, e.g., a single drop per affected eye. In aspects, about 1 drop is administered to each affected eye about two times per day.

In another aspect, the composition is administered in combination with, or comprises, an ophthalmologically effective amount of a levobunolol compound, such as levobunolol hydrochloride. In aspects, the levobunolol composition is provided in a separate solution or suspension or is combined in the composition with the latanoprost compound(s). In aspects, the levobunolol composition comprises a concentration of about 0.1-about 0.9%, e.g., between about 0.2-0.8%, between about 0.4-0.6%, or, e.g., about 0.5%, of a levobunolol compound. In aspects, the levobunolol composition is administered 1-3 times per day, such as 1 time per day, through administration of 1-2 drops per affected eye. In aspects, about 1-2 drops is administered per affected eye about once per day.

In another aspect, the composition is administered in combination with, or comprises, an ophthalmologically effective amount of a timolol compound, such as timolol hemihydrate. In aspects, the timolol composition is provided in a separate solution or suspension or is combined in the composition with the latanoprost compound(s). In aspects, the timolol composition comprises a concentration of between about 0.1-about 0.9%, e.g., between about 0.2-0.8%, between about 0.4-0.6%, or, e.g., about 0.5%, of a timolol compound. In aspects, the timolol composition is administered 1-3 times per day, such as 1 time per day, through administration of 1-2 drops, e.g., one drop per affected eye. In aspects, about 1 drop is administered per affected eye˜once per day.

In another aspect, the composition is administered in combination with, or comprises, an ophthalmologically effective amount of a Brimonidine compound, such as brimonidine tartrate. In aspects, the brimonidine composition is provided in a separate solution or suspension or is combined in the composition with the latanoprost compound(s). In aspects, the brimonidine composition comprises a concentration of between about 0.05-about 0.5%, e.g., between about 0.075-0.4%, about 0.085-0.3%, or e.g., about 0.1-0.2% of a brimonidine compound. In aspects, the brimonidine composition is administered about 1-3 times per day, such as 3 times per day, through administration of about 1-2 drops to each affected eye. In aspects, about 1 drop is administered per affected eye about three times daily.

In another aspect, the composition is administered in combination with, or comprises, an ophthalmologically effective amount of a dorzolamide compound, such as dorzolamide hydrochloride. In aspects, the dorzolamide composition is provided in a separate solution or suspension or is combined in the composition with the latanoprost compound(s). In aspects, the dorzolamide composition comprises a concentration of 0.4 to about 4%, e.g., between about 0.8-3.6%, between about 1.2-3.2%, between about 1.6-2.8%, or, e.g., such as about 2% of a dorzolamide compound. In aspects, the dorzolamide composition is administered 1-3 times per day, such as 1 time per day, through administration of 1-2, usually one drop per affected eye three times daily.

In another aspect, the composition is administered in combination with, or comprises, an ophthalmologically effective amount of an echothiophate iodide compound, such as echothiophate iodide. In aspects, the echothiophate iodide composition is provided in a separate solution or suspension or is combined in the composition with the latanoprost compound(s). In aspects, the echothiophate iodide composition comprises a concentration of between about 0.01-about 0.5%, e.g., between about 0.025-0.4%, about 0.05-0.35%, or e.g., about 0.1-0.3% of an echothiophate iodide compound. In aspects, the echothiophate iodide composition is administered 1 time every other day, or 1-2 times per day, such as 1 time per day, through administration of 1-2, usually two drops per affected eye one time per day.

In another aspect, the composition is administered in combination with, or comprises, an ophthalmologically effective amount of a pilocarpine compound, such as pilocarpine hydrochloride. In aspects, the pilocarpine composition is provided in a separate solution or suspension or is combined in the composition with the latanoprost compound(s). In aspects, the pilocarpine composition comprises a concentration of between about 0.025-about 7%, e.g., between about 0.5-6.5%, about 0.75-7%, or e.g., about 0.80-6%, e.g., between about 0.5% and 5% of a pilocarpine compound. In aspects, the pilocarpine composition is administered 1-4 times per day, such as 1 time per day, through administration of 1-2, usually one drop per affected eye up to four times per day.

In another aspect, the composition is administered in combination with, or comprises, an ophthalmologically effective amount of a latanoprostene bunod compound, such as latanoprostene bunod. In aspects, the latanoprostene bunod composition is provided in a separate solution or suspension or is combined in the composition with the latanoprost compound(s). In aspects, the latanoprostene bunod composition comprises a concentration of between about 0.01-about 0.10%, e.g., between about 0.015-0.09%, about 0.0175-0.5%, or e.g., about 0.02-0.03% of a latanoprostene bunod compound. In aspects, the latanoprostene bunod composition is administered 1-2 times per day, such as 1 time per day, through administration of 1-2, usually one drop per affected eye once per day.

In another aspect, the composition is administered in combination with, or comprises, an ophthalmologically effective amount of a tafluprost compound, such as tafluprost. In aspects, the tafluprost composition is provided in a separate solution or suspension or is combined in the composition with the latanoprost compound(s). In aspects, the tafluprost composition comprises a concentration of between about 0.0005-about 0.005%, between about 0.0007-0.004%, between about 0.0009-0.0035%, between about 0.001-0.003%, or, e.g., such as about 0.0015 of a tafluprost compound. In aspects, the tafluprost composition is administered 1-2 times per day, such as 1 time per day, through administration of 1-2, usually one drop per affected eye once per day.

In another aspect, the composition is administered in combination with, or comprises, an ophthalmologically effective amount of a travoprost compound, such as travoprost. In aspects, the travoprost composition is provided in a separate solution or suspension or is combined in the composition with the latanoprost compound(s). In aspects, the travoprost composition comprises a concentration between about 0.0005-about 0.007%, between about 0.0007-0.006%, between about 0.0009-0.005%, e.g., such as about 0.004% of a travoprost compound. In aspects, the travoprost composition is administered 1-2 times per day, such as 1 time per day, through administration of 1-2, usually one drop per affected eye once per day.

In another aspect, the composition is administered in combination with, or comprises, an ophthalmologically effective amount of a bimatoprost compound, such as bimatoprost. In aspects, the bimatoprost composition is provided in a separate solution or suspension or is combined in the composition with the latanoprost compound(s). In aspects, the bimatoprost composition comprises a concentration of between about 0.002 to about 0.02%, e.g., between about 0.004-0.018%, between about 0.006-0.0016%, between about 0.008-0.0014%, or, e.g., such as about 0.01% of a bimatoprost compound. In aspects, the bimatoprost composition is administered 1 time per day, usually one drop per affected eye once per day.

In another aspect, the composition is administered in combination with, or comprises, an ophthalmologically effective amount of a brinzolamide compound. In aspects, the brinzolamide composition is provided in a separate solution or suspension or is combined in the composition with the latanoprost compound(s). In aspects, the brinzolamide composition comprises a concentration of between about 0.02 to about 2%, e.g., between about 0.4-1.8%, between about 0.6-1.6%, between about 0.8-1.4%, or, e.g., such as about 1% of a brinzolamide compound. In aspects, the brinzolamide composition is administered about 1-3 times per day, such as 3 times per day, through administration of about 1-2 drops to each affected eye. In aspects, about 1 drop is administered per affected eye about three times daily.

In another aspect, the composition is administered in combination with, or comprises, an ophthalmologically effective amount of a netarsudil compound, such as netarsudil. In aspects, the netarsudil composition is provided in a separate solution or suspension or is combined in the composition with the latanoprost compound(s). In aspects, the netarsudil composition comprises a concentration of between about 0.005-about 0.5%, e.g., between about 0.01-0.4%, about 0.015-0.3%, or e.g., about 0.02% of a netarsudil compound. In aspects, the netarsudil composition is administered 1 time per day, usually one drop per affected eye once per day.

In another aspect, the composition is administered in combination with, or comprises, an ophthalmologically effective amount of an apraclonidine compound, such as apraclonidine. In aspects, the apraclonidine composition is provided in a separate solution or suspension or is combined in the composition with the latanoprost compound(s). In aspects, the apraclonidine composition comprises a concentration of between about 0.5%-2%, between about 0.75% and 1.5%, between about 0.85% and 1.2%, e.g., such as about 1% of an apraclonidine compound. In aspects, the apraclonidine composition is administered about 1-3 times per day, such as 3 times per day, through administration of about 1-2 drops to each affected eye. In aspects, about 1 drop is administered per affected eye about three times daily.

In aspects, the second or more APIs can be a non-prostaglandin analog antiglaucoma agent.

Additional APIs in a composition of the invention are typically present in a therapeutically effective amount, which is an amount that results in a DoS physiological or therapeutic effect in a statistically number of patients/subjects in one or more well-controlled studies. In aspects, the therapeutically effective amount is an amount lower than would be effective if the second API was present alone but is effective in view of the combination with the latanoprost compound(s) of the composition, the formulation of the composition (e.g., the inclusion of the PCCC or other components, such as BAK), or both.

In aspects, latanoprost compound-containing ophthalmologically suitable compositions described herein can comprise a second API selected from the group comprising but not limited to netarsudil or netarsudil compound or other rho kinase inhibitors.

In certain aspects, the second or more API is not bimatoprost; that is, in aspects, a combination product of the inventive composition does not comprise a combination of latanoprost and bimatoprost. According to certain aspects, compositions herein do not comprise timolol, tyloxapol, trometamol, travoprost, or any combination of some, most, ≥75% thereof, or all thereof.

In certain aspects, ophthalmic compositions described herein, comprise one or more prostaglandins as replacement(s) to/alternative(s) to latanoprost, such that the compositions are latanoprost free. In aspects, such possible alternatives to latanoprost include but may not be limited to bimatoprost, travoprost, trimoprostil, rioprostil, cloprostenol, fluprostenol, luprostiol, etiproston, tiaprost, unoprostone and its derivatives like unoprostone isopropyl, misoprostol, sulfoprostone, gemeprost, alfaprostol, delprostenate, and the like.

### Stability Characteristics of Compositions of the Invention

As used herein, the term “stable” refers to any preparation of one or more APIs, e.g., prostaglandins, such as latanoprost compounds, e.g., latanoprost, a pharmaceutically acceptable salt thereof, having sufficient physical and chemical stability to allow storage at a convenient temperature, such as between about 0° C. and about 50° C., for a commercially reasonable period of time (e.g., up to about 36 months or longer). The term “physical stability” refers to the ability of the composition to maintain its original color (e.g., retain its color immediately after completion of manufacturing), dissolved oxygen level, head space oxygen level, particulate matter, and combinations thereof. The term “chemical stability” relates to formation of drug-related impurities in terms of total impurity, single maximum individual impurity, and maximum individual unknown impurity. For the purpose of the present invention, chemical stability also includes maintenance of pH of the finished formulation. In aspects, “stability” can be indicated by, e.g., an assay of latanoprost (e.g., an indication of loss of or prevention of loss of latanoprost compound(s)), an assay of related latanoprost compounds, e.g., one or more compounds related to latanoprost, a measure of one or more impurities, measurement of pH, measurement of osmolality, or any combination thereof, or, e.g., by other methods of stability known in the art.

According to aspects, the compositions herein provide latanoprost ophthalmic compositions, which in aspects can be used to treat elevated intra-ocular pressure or glaucoma, wherein the composition/formulation can be stored under room temperature conditions and/or accelerated stability testing conditions. In aspects, ophthalmologically suitable compositions described herein comprising a polyoxyl-40 hydrogenated castor oil composition (PCCC) provides for a formulation which is stable at room temperature and/or under accelerated stability testing conditions.

In aspects, the ophthalmologically suitable compositions of the invention are capable of retaining at least about 90%, ≥˜91%, ≥˜92%, ≥˜93%, ≥˜94%, ≥˜95%, ≥˜96%, ≥˜97%, ≥˜98%, ≥˜99%, or even greater of their initial latanoprost compound composition content when stored at about 22° C.-about 25° C. and about 60% relative humidity for a period of at least about 3 months, such as at least about 2, ˜3, ˜4, ˜6, ˜8, ˜10, ˜12, or at least about ˜18 months, such as, e.g., ˜24, ˜30, or at least about 36 months. In specific aspects, the compositions of the invention comprise about 0.001% of an ophthalmologically suitable latanoprost compound composition and about 0.1% to about 5.0% of a polyoxyl castor oil composition, wherein the composition retains ≥˜95%, ≥˜96%, ≥˜97%, ≥˜98%, ≥˜99%, or even greater of the initial latanoprost compound composition content when stored at about 22° C.-about 25° C. and about 60% relative humidity for a period of at least about 3 months, at least about 4 months, at least about 5 months, or for example for at least about 6 months. In aspects, the ophthalmologically suitable compositions herein can retain at least about 99% of the initial latanoprost compound composition content when stored at about 40° C. for a period of at least about 1 month.

According to certain aspects, the compositions of the invention described herein are capable of being stored at temperatures of greater than 8° C. during all stages of product manufacturing, storage, and use, including for time periods commercially relevant after manufacturing, such as for example for about 6, ˜12, ˜18, ˜24, or -36 months (for pharmaceutical products, stability can be required for commercially relevant times after manufacturing which can extend out to 36 months or longer, during which time a product can be kept in its original packaging under specified storage conditions.) According to certain aspects, the compositions of the invention described herein are capable of being stored at ambient temperatures, e.g., temperatures of about 20-about 40° C., during all stages of product manufacturing, storage, and use, including for time periods commercially relevant after manufacturing, such as for example for about 6, ˜12, ˜18, ˜24, or ˜36 months. This contrasts with, for example, the currently marketed XALATAN® ophthalmic solution, which lacks stability at ambient or room temperature and requires the product to be stored under refrigerated conditions. Specifically, the recommended storage conditions for XALATAN® include storage under refrigeration at a temperature of approximately 2-8° C. as an unopened bottle, while during shipment to the patient, the bottle may be maintained at temperature up to 40° C. for a period not exceeding 8 days. Once the bottle is opened for use, XALATAN® may be stored at room temperature up to 25° C. for a period of 6 weeks.

In certain aspects, compositions of the present invention can maintain physical and chemical stability when stored at ambient temperatures, ranging anywhere between about 0° C. and about 50° C., for a period longer than 8 days, 2 weeks, 1 month, or 6 weeks. In aspects, the compositions of the present invention can remain stable for periods of time at ambient temperature significantly (e.g., for a statistically significant period) longer than that of current on-market products such as XALATAN® or XELPROS®.

In aspects, the inventive ophthalmologically suitable compositions are capable of maintaining their potency, their dissolution profile, or both, within at least about 10%, within at least ˜8%, within at least ˜6%, within at least ˜4%, or even within at least ˜2% of their values at the point at which they are considered a finished product post-manufacturing, over a period of at least about 1 month, at least about 2 months, at least about 6 months, at least about 12 months, at least about 18 months, at least about 24 months, at least about 28 months, or for at least about 36 months or longer when stored under ambient conditions, such as, e.g., between about 0° C.-50° C. In aspects such a term can be referred to as the shelf life of the compositions.

In aspects, the ophthalmologically suitable compositions of the invention comprise a PCCC which is detectably or significantly more effective at stabilizing one or more latanoprost compounds of the composition (e.g., latanoprost) than polysorbate 80. That is, in aspects, the invention comprises a PCCC which is capable of stabilizing, e.g., latanoprost, for a detectably or significantly longer period at ambient storage conditions, e.g., 0° C.-50° C., or at accelerated stability study conditions, than similar compositions comprising polysorbate 80 in lieu of a PCCC.

In aspects, compositions provided by the invention demonstrate the ability to maintain on average an amount of latanoprost compound which is within at least about 10% of an initial amount of latanoprost compound when the composition is stored for a period of about 1 month, about 2 months, about 3 months, or about 6 months, at 40° C.±2° C. and not more than 25% relative humidity, at 25° C.±2° C. and 40%±5% relative humidity, or, e.g., between about 2° C. to about 8° C., such as an amount of latanoprost compound which is within at least ˜9.5%, ˜9%, ˜8.5%, ˜8%, ˜7.5%, ˜7%, ˜6.5%, ˜6%, ˜5.5%, ˜5%, ˜4.5%, ˜4%, ˜3.5%, ˜3%, ˜2.5%, ˜2%, ˜1.5%, ˜1%, ˜0.5%, or, e.g., ˜0.1% of an initial amount of latanoprost compound (e.g., ˜0.1%-˜8%, ˜0.1%-˜6%, ˜0.1%-˜4%, or, e.g., ˜0.1-˜2%, such as, e.g., ˜1%-˜6%, e.g., ˜1%-˜3% or, e.g., ˜4%-˜6%) when the composition is stored for a period of about 1 month, about 2 months, about 3 months, or about 6 months, at 40° C.±2° C. and not more than 25% relative humidity, at 25° C.±2° C. and 40%±5% relative humidity, or, e.g., between about 2° C. to about 8° C. In aspects, compositions stored in containers sterilized by gamma sterilization (e.g., gamma ray sterilization) demonstrate the ability to maintain on average an amount of latanoprost compound (as compared to an initial amount) which is detectably or significantly greater than the amount of latanoprost compound that is maintained when the same composition is stored in containers sterilized by ethylene oxide.

In aspects, compositions provided by the invention demonstrate the ability to maintain on average an amount of benzalkonium chloride (BKC) which is within at least about 10% of an initial amount of BKC when the composition is stored for a period of about 1 month, about 2 months, about 3 months, or about 6 months, at 40° C.±2° C. and not more than 25% relative humidity, at 25° C.±2° C. and 40%±5% relative humidity, or, e.g., between about 2° C. to about 8° C., such as an amount of BKC which is within at least ˜9.5%, ˜9%, ˜8.5%, ˜8%, ˜7.5%, ˜7%, ˜6.5%, ˜6%, ˜5.5%, ˜5%, ˜4.5%, ˜4%, ˜3.5%, ˜3%, ˜2.5%, ˜2%, ˜1.5%, ˜1%, ˜0.5%, or, e.g., ˜0.1% of an initial amount of BKC (e.g., ˜0.1%-˜8%, ˜0.1%-˜6%, ˜0.1%-˜4%, or, e.g., ˜0.1-˜2%, such as, e.g., ˜1%-˜3% when the composition is stored for a period of about 1 month, about 2 months, about 3 months, or about 6 months, at 40° C.±2° C. and not more than 25% relative humidity, at 25° C.±2° C. and 40%±5% relative humidity, or, e.g., between about 2° C. to about 8° C. In aspects, compositions stored in containers sterilized by gamma sterilization demonstrate the ability to maintain on average an amount of BKC (as compared to an initial amount) which is at least similar to (e.g., for example, within 20% or within 10% of, or, e.g., within about 5% of, on average) the amount of BKC that is maintained when the same composition is stored in containers sterilized by ethylene oxide, or, e.g., is detectably or significantly greater when stored in a container sterilized by gamma sterilization compared to the same composition stored in a container sterilized by ethylene oxide.

In aspects, the ratio of the amount of one or more latanoprost compound(s), e.g., a single latanoprost compound or composition of latanoprost compounds in the composition to the amount of benzalkonium chloride remains relatively stable when a composition provided by the invention is stored for a period of about 1 month, about 2 months, about 3 months, or about 6 months, at 40° C.±2° C. and not more than 25% relative humidity, at 25° C.±2° C. and 40%±5% relative humidity, or, e.g., when stored between about 2° C. to about 8° C.

In aspects, compositions provided by the invention demonstrate the ability to maintain a level of impurity(ies) of below 5% when stored for a period of about 1 month, about 2 months, about 3 months, or about 6 months, at 40° C.±2° C. and not more than 25% relative humidity, at 25° C.±2° C. and 40%±5% relative humidity, or, e.g., when stored between about 2° C. to about 8° C. In aspects, compositions provided by the invention demonstrate the ability to maintain a level of impurity(ies) of below 5% when stored for a period of about 1 month, about 2 months, about 3 months, or about 6 months, at 40° C.±2° C. and not more than 25% relative humidity, at 25° C.±2° C. and 40%±5% relative humidity, or, e.g., when stored between about 2° C. to about 8° C. when stored in containers sterilized by gamma sterilization, when the composition is stored in containers sterilized by ethylene oxide, or when the composition is stored in either containers sterilized by gamma sterilization or ethylene oxide sterilization.

In aspects, compositions provided by the invention demonstrate the ability to maintain a pH of within about 5%, such as, e.g., within about 4.5%, ˜4%, ˜3.5%, ˜3%, ˜2.5%, ˜2%, ˜1.5%, ˜1%, ˜0.5%, or, e.g., within ˜0.1% when stored for a period of about 1 month, about 2 months, about 3 months, or about 6 months, at 40° C.±2° C. and not more than 25% relative humidity, at 25° C.±2° C. and 40%±5% relative humidity, or, e.g., when stored between about 2° C. to about 8° C. In aspects, demonstrate the ability to maintain a pH of within about 5%, such as, e.g., within about 4.5%, ˜4%, ˜3.5%, ˜3%, ˜2.5%, ˜2%, ˜1.5%, ˜1%, ˜0.5%, or, e.g., within ˜0.1% when stored for a period of about 1 month, about 2 months, about 3 months, or about 6 months, at 40° C.±2° C. and not more than 25% relative humidity, at 25° C.±2° C. and 40%±5% relative humidity, or, e.g., when stored between about 2° C. to about 8° C. when the composition is stored in containers sterilized by gamma sterilization, when the composition is stored in containers sterilized by ethylene oxide, or when the composition is stored in either containers sterilized by gamma sterilization or ethylene oxide sterilization.

In aspects, compositions provided by the invention demonstrate the ability to maintain an osmolality of within about 10%, such as within about 9%, ˜8%, ˜7%, ˜6%, or ˜5%, such as, e.g., within about ˜4%, ˜3%, ˜2%, ˜1%, ˜0.5%, or, e.g., within ˜0.1% when stored for a period of about 1 month, about 2 months, about 3 months, or about 6 months, at 40° C.±2° C. and not more than 25% relative humidity, at 25° C.±2° C. and 40%±5% relative humidity, or, e.g., when stored between about 2° C. to about 8° C. In aspects, compositions provided by the invention demonstrate the ability to maintain an osmolality of within about 10%, such as within about 9%, ˜8%, ˜7%, ˜6%, or ˜5%, such as, e.g., within about ˜4%, ˜3%, ˜2%, ˜1%, ˜0.5%, or, e.g., within ˜0.1% when stored for a period of about 1 month, about 2 months, about 3 months, or about 6 months, at 40° C.±2° C. and not more than 25% relative humidity, at 25° C.±2° C. and 40%±5% relative humidity, or, e.g., when stored between about 2° C. to about 8° C. when the composition is stored in containers sterilized by gamma sterilization, when the composition is stored in containers sterilized by ethylene oxide, or when the composition is stored in either containers sterilized by gamma sterilization or ethylene oxide sterilization.

### Preparation of Ophthalmologically Suitable Formulations

According to aspects, the invention is a process for preparation an ophthalmic composition comprising latanoprost in an amount of about 0.001% to about 0.01% by weight, a polyoxyl 40 hydrogenated castor oil composition (PCCC) in an amount of about 0.1% to about 5.0% by weight, benzalkonium chloride in an amount of about 0.0% to about 0.02% by weight, sodium chloride in a concentration of about 0.1% to about 0.5%, and monobasic and or dibasic sodium phosphate in a concentration of between about 0.05 to 1.0% and water, wherein the composition remains stable when stored at room temperature conditions and/or accelerated stability testing conditions.

In aspects, compositions of the invention can be manufactured using a process comprising (a), in a suitable container, taking the required amount of PCCC and adding approximately 25% of the batch weight of water for injection (WFI) to the PCCC, followed by stirring; (b) adding the required amount of benzalkonium chloride, if BKC is to be a component of the composition, and ensuring thorough dissolution of the mixture; (c) stirring contents slowly to avoid any potential foaming; (d) adding the required amount of latanoprost compound and ensuring its dissolution; (e) adding WFI so that batch weight is about 80% of the target; (f) adding remaining ingredients, such as one or more additional excipients, one after the other, making sure that the previous ingredient is in solution prior to the addition of the next ingredient; (g) filtering the solution through suitable filter and transferring it to a suitable sterile tank; (h) packaging the product aseptically in previously sterilized ophthalmic containers; (i) as appropriate for the containers being used, inserting the tip of the container and capping the product; (j) inspecting the bottles for extraneous particles; and (k) labeling the bottles.

The compositions disclosed herein can comprise the latanoprost and one or more of the listed excipients, can consist essentially of the latanoprost and one or more of the listed excipients, or can consist of the latanoprost and one or more of the listed excipients. Such compositions can be formulated similarly.

In aspects, the compositions herein are sterilized prior to or upon packaging. In certain aspects, any suitable sterilization method can be used. In certain aspects, the sterilization is carried out using one or more methods selected from one or more of heat sterilization, gaseous sterilization, filtration sterilization or radiation sterilization.

In aspects, compositions provided by the invention are packaged in pre-sterilized containers. In aspects, such containers are sterilized via ethylene oxide (ETO) sterilization. In aspects, such containers are sterilized via gamma sterilization. In aspects, compositions stored in containers sterilized by ETO demonstrate at least similar stability to compositions stored in containers sterilized using gamma sterilization when stored under one or more conditions of a stability study (e.g., the stability study described in, e.g., Example 2 herein, such as, e.g., 40° C.±2° C. and not more than (NMT) 25% relative humidity (RH); 25° C.±2° C. and 40%±5% relative humidity (RH); or, e.g., 2° C.-8° C. In aspects, compositions stored in containers sterilized by ETO demonstrate detectably or significantly greater stability to compositions stored in containers sterilized using gamma sterilization when stored under one or more conditions of a stability study (e.g., the stability study described in, e.g., Example 2 herein, such as, e.g., 40° C.±2° C. and not more than (NMT) 25% relative humidity (RH); 25° C.±2° C. and 40%±5% relative humidity (RH); or, e.g., 2° C.-8° C. In aspects, compositions stored in containers sterilized by gamma sterilization demonstrate detectably or significantly greater stability to compositions stored in containers sterilized using ETO sterilization when stored under one or more conditions of a stability study (e.g., the stability study described in, e.g., Example 2 herein, such as, e.g., 40° C.±2° C. and not more than (NMT) 25% relative humidity (RH); 25° C.±2° C. and 40%±5% relative humidity (RH); or, e.g., 2° C.-8° C. In aspects, compositions stored in ETO sterilized containers and gamma sterilized containers do not demonstrate a difference in stability which is greater than 15% (e.g., one demonstrating a stability which is 15% greater than the other), e.g., not greater than 10%, not greater than 5%, not greater than 3%, or, e.g., not greater than about 2.5% than the other. In aspects, compositions stored in containers sterilized by ETO demonstrate a stability which is at least ˜0.1%, ˜0.5%, ˜1%, or, e.g., at least ˜3%, at least ˜5%, at least about 10%, or more, greater than that of compositions stored in containers sterilized by gamma sterilization. In aspects, compositions stored in containers sterilized by gamma sterilization demonstrate a stability which is at least ˜0.1%, ˜0.5%, ˜1%, or, e.g., at least ˜3%, at least ˜5%, at least about 10%, or more, greater than that of compositions stored in containers sterilized by ETO sterilization.

### Therapeutic Applications

In a general aspect, the invention provides a method for modulating the physiological conditions of the eye in a mammalian subject comprising administering a therapeutically effective amount of an ophthalmologically suitable composition described herein.

According to aspects, the present invention provides methods for treating glaucoma and related eye conditions, which comprises administering to a subject in need of such treatment, ophthalmologically suitable latanoprost compositions described herein. Potential damage that is caused by glaucoma and related eye conditions is irreversible, thus making the treatment of the underlying symptoms/conditions critical. In aspects, treatments described herein are capable of reducing the intraocular pressure within the eye(s). In aspects, the compositions described herein can be a first line treatment for pressure reduction, and can, in aspects, be provided as an eye drop formulation. In some aspects, for example, the compositions are capable of increasing the aqueous humor in the eye (reducing pressure), reducing fluid production in the eye (reducing pressure), or both.

In aspects, “treating” or “treatment” as used herein broadly includes any approach for obtaining beneficial or desired results in a subject's condition, such as, e.g., a detectable or significant reduction in the underlying causes, symptoms, or negative conditions associated with glaucoma or related eye conditions, such as IOP. In aspects, beneficial or desired clinical results can include, but are not limited to alleviation or amelioration of one or more symptoms or conditions, diminishment of the extent of a disease, stabilizing (i.e., not worsening) the state of disease, or delay or slowing of disease progression, amelioration, or palliation of the disease state.

According to aspects, the ophthalmologically suitable latanoprost compositions are suitable for use in the reduction of elevated intraocular pressure in patients who have been diagnosed with, for example, open-angle glaucoma or ocular hypertension. In aspects, the invention herein is a method for modulating the physiological conditions of the eye in a mammalian subject, such a method useful in, e.g., treating eye-related conditions, such as any one or more conditions described herein. In aspects, the invention comprises use of one or more inventive compositions described herein.

In certain aspects, treatment of elevated intraocular pressure with a composition described herein DOS reduces the risk of glaucomatous field loss, optic nerve damage, visual field loss, or any combination thereof. In aspects, treatment with a composition herein for a period of approximately 6 months can provide a DOS reduction in intraocular pressure, such as, for example, a reduction of at least about 10%, at least ˜12%, at least ˜14%, at least ˜16%, at least ˜18%, at least ˜20%, at least ˜22%, at least ˜24%, at least ˜26%, at least ˜28%, at least ˜30%, at least about 32%, or even more, such as at least about 33% or more (such as, for example, in patients having a baseline intraocular pressure of between about 24-25 mmHg experiencing a reduction in intraocular pressure of between about 6-8 mmHg when treated for about 6 months.)

In aspects, the inventive ophthalmologically suitable compositions are used in the treatment of elevated intraocular pressure caused by angle-closure glaucoma that has been treated with peripheral iridotomy or laser iridoplasty. In aspects, the compositions herein are capable of detectibly or significantly reducing intraocular pressure resulting from angle-closure glaucoma that has been treated with peripheral iridotomy or laser iridoplasty, such as by reducing such intraocular pressure by at least ˜1%, ˜3%, ˜5%, ˜10% or more.

In aspects, the inventive ophthalmologically suitable compositions are used to decrease intraocular pressure by increasing uveoscleral outflow. In aspects, the compositions of the invention can increase uveoscleral outflow by at least about 1%, at least ˜2%, at least about 3%, at least ˜4%, at least ˜5%, at least ˜6%, at least ˜7%, at least ˜8%, at least ˜9%, or at least about 10% or more, such as at least ˜12%, at least ˜14%, at least ˜16%, at least ˜18%, at least ˜20% or even more, in patients receiving treatment with the compositions described herein over untreated patients or can increase uveoscleral outflow by at least about 1%, at least ˜2%, at least about 3%, at least ˜4%, at least ˜5%, at least ˜6%, at least ˜7%, at least ˜8%, at least ˜9%, or at least about 10% or more, such as at least ˜12%, at least ˜14%, at least ˜16%, at least ˜18%, at least ˜20% or even more, in patients receiving treatment over the starting (baseline) levels of uveoscleral outflow of recipient patients prior to receiving treatment.

In some respects, the ophthalmologically suitable compositions described herein are capable of DOS remodeling the extracellular matrix and regulating matrix metalloproteinases. In aspects, the DOS remodeling of the extracellular matrix, the regulation of matrix metalloproteinases, or both DOS increase tissue permeability related to humor outflow pathways, DOS change outflow resistance, DOS outflow rates, or any combination thereof.

In aspects, the invention is a method for modulating the physiological conditions of the eye in a mammalian subject comprising administering a therapeutically effective amount of an ophthalmologically suitable composition described herein. In aspects, the treated subject is a human patient diagnosed with one or both conditions.

In aspects, methods of the invention comprise administering one drop of an effective amount of an ophthalmologically suitable composition from a container adapted to administer eye drops per day to treat one or more eye-related conditions as described herein, wherein each drop contains about 1.5 μg of one or more latanoprost compounds. In aspects, the average volume of each drop of the ophthalmologically suitable composition is between about 20 μL and about 70 μL, such as between about 30 μL and about 60 μL, e.g., between about 40 μL to about 50 μL.

In aspects, the methods for modulating the physiological conditions of the eye in a mammalian subject, treating one or more eye-related conditions, or both, with a composition described herein comprises shipping the composition packaged in an eye drop container (e.g., a dropper bottle) at temperatures above about 8° C., such as above ˜ 9° C., above about 10° C., above about 11° C., above about 12° C., above about 13° C., above about 14° C., or above about 15° C. or even higher. In aspects, a method of administration of the invention comprises administering the product after the product has been stored at a temperature of greater than about 30° C., such as greater than about 32° C., greater than about 35° C., or, e.g., greater than about 40° C. for a period of more than 10 days, such as more than 2 weeks, or more than about 1 month. In aspects, a method of the invention comprises continuing to administer the ophthalmologically suitable composition after the container has been opened and stored at temperature of greater than about 20° C. (e.g., greater than ˜22° C., 24° C., ˜26° C., ˜28° C., or greater than ˜30° C. for period of about 8 weeks or longer, such as a period of ˜6 weeks, ˜7 weeks, ˜8 weeks, ˜9 weeks, ˜10 weeks, ˜12 weeks, ˜16 weeks, ˜20 weeks or even longer.

In aspects, the invention is a method for treating open-angle glaucoma, reducing intraocular pressure, or both, using an ophthalmologically suitable composition described herein.

According to some aspects, inventive ophthalmologically suitable compositions of the invention can decrease intraocular pressure within about 6 hours post-administration, within about 5 hours post-administration, within about 4 hours post-administration, within about 3 hours, post-administration, or within even about 2 hours post-administration, such as between about 2-6 hours post administration or, e.g., within about 3-4 hours post-administration. In aspects, a peak decrease in reduction in intraocular pressure can be seen in recipients receiving treatment with the compositions herein within about 16 hours, within ˜14, hours, within ˜12 hours, within ˜10 hours, within ˜8 hours, or, e.g., within ˜6 hours, such as, e.g., within between about 6-14 hours or within between about 8-12 hours post-administration.

According to certain aspects, the Cmax of a latanoprost compound of the present compositions, e.g., of latanoprost, in the systemic circulation is reached within approximately 10 minutes of administration, such as within approximately 8 minutes, approximately 6 minutes, approximately 4 minutes, or approximately 2 minutes of administration. In aspects, the Cmax of latanoprost in the systemic circulation can be reached between approximately 4 minutes and 6 minutes after administration, such as at about 5 minutes after administration. In aspects, the Cmax in the aqueous humor is attained within about 4 hours, such as within ˜3.5 hours, ˜3 hours, ˜2.5 hours, about 2 hours, about 1.5 hours, or within about 1 hour of administration, such as within about 1-3 hours after administration, e.g., within about 2 hours after administration.

In aspects, the intraocular pressure decreases attained by a patient receiving the compositions herein and affected by the compositions herein, e.g., affected by a single dose of compositions herein, can be maintained over the course of an extended period, such as over the course of at least about 6 hours, at least about 12 hours, at least about 18 hours, at least about 24 hours, or even more, prior to the administration of the next dose of composition.

In aspects, the invention comprises a method for modulating the physiological conditions of the eye in a mammalian subject, wherein the method comprises administering the ophthalmologically suitable composition to the subject for a period of at least six months with an average decrease in intraocular pressure (IOP) of at least 15% throughout or at the end of the treatment period with a significant number of subjects exhibiting a decrease of IOP of at least 20%, 25%, or 30%, throughout or at the end of the treatment period.

In aspects, the ophthalmic compositions herein can be applied once or twice in the affected eye(s). In aspects, the therapeutic benefits described herein can be obtained when the ophthalmic compositions are administered once per day. In aspects, the therapeutic benefits described herein can be obtain when the ophthalmic compositions are administered twice per day.

In aspects, a recommended dose for the ophthalmologically suitable compositions herein is approximately 1 drop (e.g., a drop comprising approximately 1.5 μg of API, e.g., one drop comprising approximately 1.5 μg of latanoprost) in the affected eye(s) once per day. In aspects it is recommended to administer the product in the evening.

In aspects, treatment protocols comprising the compositions herein are such that if one dose is missed, the recipient is advised to resume treatment with the next scheduled dose, skipping the missed dose and not double-dosing or taking the missed dose within a period during which two doses would not normally be administered. In aspects, dosing should occur no more than once per 24-hour period.

In aspects, compositions herein should not be combined with other ophthalmological products comprising one or more additional prostaglandins or prostaglandin analogues. In certain aspects, the compositions of the invention can be used concomitantly with other topical ophthalmic drug products not comprising prostaglandins to lower intraocular pressure. In aspects, if the compositions herein are used in combination with one or more additional topical ophthalmic drugs, the two drugs should be administered at least 5 minutes apart, such as at least about 5 min, at least about 10 min, at least about 15 min, at least about 20 min, at least about 25 min, or at least about 30 min apart or more.

According to aspects, the invention herein is a method of treating and preventing the ophthalmic diseases by administering an ophthalmic composition comprising latanoprost (e.g., the inventive compositions described herein) and a second antiglaucoma agent such as, for example, beta blocker(s), carbonic anhydrase inhibitor(s), or alpha-adrenergic agonist(s), wherein the inventive composition remains stable when stored at room temperature conditions and/or accelerated stability conditions. Exemplary beta blockers include but may not be limited to timolol, betaxolol, carteolol, levobunolol, befunolol, metipranolol and mepindolol. Exemplary carbonic anhydrase inhibitors include but may not be limited to brinzolamide acetazolamide, methazolamide, dorzolamide, diclofenamide, ethoxzolamide, and zonisamide. Alpha-adrenergic agonist(s) can include but may not limited to brimonidine, clonidine, apraclonidine, dexmedetomidine and fadolmidine.

In aspects, a second antiglaucoma agent can be administered with a latanoprost compound as a combination composition. In aspects, methods of modulating the physiological conditions of the eye in a mammalian subject can comprise administration of a second antiglaucoma agent that is administered separately from the ophthalmologically suitable composition and the timing of administration of the ophthalmologically suitable composition and the second antiglaucoma agent is separated by at least about 5 minutes.

In certain aspects, the invention comprises the use of any composition described herein or incorporating any of the conditions suitable for treatment with a composition described herein, in the preparation of a medicament for treating open-angle glaucoma, reducing intraocular pressure, or both.

According to certain aspects, ophthalmologically suitable composition(s) provided by the invention, when used in a method of reducing intraocular pressure (e.g., elevated IOP associated with glaucoma, e.g., open-angle glaucoma), such methods being described herein and when administered in effective amount(s), demonstrate a reduction in intraocular pressure when administered to a mammalian eye suffering from elevated intraocular pressure which is at least statistically equivalent to, or, e.g., in aspects, is better than (e.g., the reduction is greater than) that achieved by administration of Xalatan® (the product approved by the US-FDA under NDA number 020597) when administered for the same condition for the same period of time.

In aspects, ophthalmologically suitable composition(s) provided by the invention, when used in a method of reducing intraocular pressure (e.g., elevated IOP associated with glaucoma, e.g., open-angle glaucoma), such methods being described herein and when administered in effective amount(s), demonstrate a mean percent change in intraocular pressure over the course of a 24-hour time period after a single administration which is statistically equivalent to that demonstrated by Xalatan® (the product approved by the US-FDA under NDA number 020597) when administered for the same condition for the same period of time.

In aspects, one or more compositions provided by the invention described herein are capable of demonstrating a maximum reduction in intraocular pressure over the course of 24 hours after a single administration of at least about 2 mmHg, such as at least about 3 mmHg, at least about 4 mmHg, or at least about 5 mmHg

In aspects, one or more compositions provided by the invention described herein are capable of demonstrating a percent change, e.g., a percent reduction, in intraocular pressure over the course of 24 hours after a single administration of at least about 5%, at least about 7%, at least about 10%, at least about 12%, or, e.g., at least about 15%, such as, e.g., at least about 1% to about 30%, ˜5%-˜25%, ˜5%-˜20%, ˜5%-˜15%, or, e.g., ˜5%-˜10%. In aspects, one or more compositions provided by the invention described herein are capable of demonstrating a mean percent change (decrease) in intraocular pressure over the course of a 24-hour time period after a single administration which is at least about 2%, at least about 2.5%, at least about 3%, at least about 3.5%, at least about 4%, at least about 4.5%, or, e.g., at least about 5% such as, e.g., at least about 1% to about 20%, ˜2%-˜15%, ˜2%-˜10%, or, e.g., ˜2%-˜5%.

### Exemplary Aspects of the Invention

The following is a non-limiting listing of exemplary aspects of the invention. This section specifically presents aspects of the invention in an alternative disclosure format, to better illuminate the scope of the invention (using several cross-referenced paragraphs that list particular aspects to link selected aspects and thereby show relationships between such facets of the invention). These aspects also or alternatively can be combined with any aspect of the invention described in any other section of this disclosure and vice versa.

In aspects, the invention provides an ophthalmologically suitable composition comprising about 0.001% to about 0.01% of an ophthalmologically suitable latanoprost compound composition and about 0.1% to about 5.0% of a polyoxyl castor oil composition, wherein the composition retains at least 98% of the initial latanoprost compound composition content when stored at about 22° C.-about 25° C. and about 60% relative humidity for a period of at least about 3 months (aspect 1).

In aspects, the invention provides the ophthalmologically suitable composition described in aspect 1, wherein the polyoxyl castor oil composition primarily comprises a polyoxyl castor oil compound comprising about 30-about 60 oxyethylene units (aspect 2).

In aspects, the invention provides the ophthalmologically suitable composition described in aspect 1 or aspect 2, wherein the polyoxyl castor oil composition primarily comprises hydrogenated polyoxyl castor oil compound(s) (aspect 3).

In aspects, the invention provides the ophthalmologically suitable composition described in aspect 3, wherein at least 75% of the polyoxyl castor oil compound(s) in the polyoxyl castor oil composition are hydrogenated (aspect 4).

In aspects, the invention provides the ophthalmologically suitable composition described in aspect 4, wherein essentially all the polyoxyl castor oil compound(s) in the polyoxyl castor oil composition are hydrogenated (aspect 5).

In aspects, the invention provides the ophthalmologically suitable composition described in aspect 5, wherein all detectable polyoxyl castor oil compound(s) in the polyoxyl castor oil composition are hydrogenated (aspect 6).

In aspects, the invention provides the ophthalmologically suitable composition described in any one or more of aspects 3-6, wherein the polyoxyl hydrogenated castor oil composition primarily comprises a polyoxyl hydrogenated castor oil compound comprising 30-60, e.g., about 35-50 oxyethylene units (aspect 7).

In aspects, the invention provides the ophthalmologically suitable composition described in aspect 7, wherein at least 65% of the polyoxyl hydrogenated castor oil compound(s) in the hydrogenated castor oil composition comprise 40-60, e.g., about 35-50 oxyethylene units (aspect 8).

In aspects, the invention provides the ophthalmologically suitable composition described in aspect 8, wherein essentially all the polyoxyl hydrogenated castor oil compound(s) in the hydrogenated castor oil composition comprise 40-60, e.g., about 35-50 oxyethylene units (aspect 9).

In aspects, the invention provides the ophthalmologically suitable composition described in aspect 9, wherein all detectable polyoxyl hydrogenated castor oil compound(s) in the composition comprise 40-60, e.g., about 35-50 oxyethylene units (aspect 10).

In aspects, the invention provides the ophthalmologically suitable composition described in any one or more of aspects 7-10, wherein the polyoxyl hydrogenated castor oil composition primarily comprises a polyoxyl hydrogenated castor oil compound comprising about 32-52 oxyethylene units (e.g., 35-50 oxyethylene units) (aspect 11).

In aspects, the invention provides the ophthalmologically suitable composition described in aspect 11, wherein at least 65% of the polyoxyl hydrogenated castor oil composition is composed of a polyoxyl hydrogenated castor oil compound comprising about 35-50 oxyethylene units (aspect 12).

In aspects, the invention provides the ophthalmologically suitable composition described in aspect 12, wherein the polyoxyl hydrogenated castor oil compound makes up 65-85% of the polyoxyl hydrogenated castor oil composition (aspect 13).

In aspects, the invention provides the ophthalmologically suitable composition described in aspect 12 or aspect 13, wherein at least about 10% of the polyoxyl hydrogenated castor oil composition is composed of one or more hydrophilic compounds (aspect 14).

In aspects, the invention provides the ophthalmologically suitable composition described in aspect 14, wherein 15-35% of the polyoxyl hydrogenated castor oil composition is at least primarily composed of polyethylene glycol, glycerol ethoxylates, or a mixture thereof (aspect 15).

In aspects, the invention provides the ophthalmologically suitable composition described in aspect 15, wherein 15-35% of the polyoxyl hydrogenated castor oil composition consists essentially of a mixture of polyethylene glycol and glycerol ethoxylates (aspect 16).

In aspects, the invention provides the ophthalmologically suitable composition according to any one or more of aspects 2-16, wherein the polyoxyl hydrogenated castor oil compound composition makes up about 0.25%-about 2.5% of the composition (aspect 17).

In aspects, the invention provides the ophthalmologically suitable composition according to aspect 17, wherein the polyoxyl hydrogenated castor oil compound composition makes up about 0.3%-about 1.5% of the composition (aspect 18).

In aspects, the invention provides the ophthalmologically suitable composition according to aspect 18, wherein the polyoxyl hydrogenated castor oil compound composition makes up about 0.5%-about 1.5% of the composition (aspect 19).

In aspects, the invention provides the ophthalmologically suitable composition according to aspect 19, wherein the polyoxyl hydrogenated castor oil compound composition makes up about 0.75%-about 2.25% or about 0.75%-about 1.25% of the composition (aspect 20).

In aspects, the invention provides the ophthalmologically suitable composition according to aspect 20, wherein the polyoxyl hydrogenated castor oil compound composition makes up about 1% of the composition (aspect 21).

In aspects, the invention provides the ophthalmologically suitable composition according to any one or more of aspects 1-21, wherein the polyoxyl hydrogenated castor oil compound composition is the primary non-ionic surfactant in the composition (aspect 22).

In aspects, the invention provides the ophthalmologically suitable composition according to aspect 22, wherein the polyoxyl hydrogenated castor oil composition makes up at least about 75% of the non-ionic surfactant content of the composition (aspect 23).

In aspects, the invention provides the ophthalmologically suitable composition according to aspect 23, wherein the polyoxyl hydrogenated castor oil composition is essentially the only non-ionic surfactant content of the composition (aspect 24).

In aspects, the invention provides the ophthalmologically suitable composition according to aspect 24, wherein the polyoxyl hydrogenated castor oil composition is the only detectable non-ionic surfactant component of the composition (aspect 25).

In aspects, the invention provides the ophthalmologically suitable composition described in any one or more of aspects 1-25, wherein the polyoxyl castor oil composition is detectably more effective at stabilizing the latanoprost compound than polysorbate 80 (aspect 26).

In aspects, the invention provides the ophthalmologically suitable composition described in any one or more of aspects 1-26, wherein the latanoprost compound composition is present in an amount of about 0.0025% to about 0.0075% (aspect 27).

In aspects, the invention provides the ophthalmologically suitable composition described in aspect 27, wherein the latanoprost compound composition is present in an amount of about 0.005% (aspect 28).

In aspects, the invention provides the ophthalmologically suitable composition according to any one or more of aspects 1-28, wherein at least about 75% of the ophthalmologically suitable composition is water (aspect 29).

In aspects, the invention provides the ophthalmologically suitable composition according to any one or more of aspects 1-29, wherein the composition comprises a preservative-enhancing or preservative-effective amount of benzalkonium chloride in a concentration of less than 0.2 mg/mL (aspect 30).

In aspects, the invention provides the ophthalmologically suitable composition according to any one or more of aspects 1-30, wherein the composition is free of benzalkonium chloride (aspect 31).

In aspects, the invention provides the ophthalmologically suitable composition according to any one or more of aspects 1-31, wherein the composition retains at least 97% of the initial latanoprost compound composition content when stored at about 22° C.-about 25° C. and about 60% relative humidity for a period of at least about 6 months (aspect 32).

In aspects, the invention provides the ophthalmologically suitable composition according to any one or more of aspects 1-32, wherein the composition retains at least 99% of the initial latanoprost compound composition content when stored at about 40° C. for a period of at least about 1 week (aspect 33).

In aspects, the invention provides the ophthalmologically suitable composition according to any one or more of aspects 1-33, wherein the composition retains at least 99% of the initial latanoprost compound composition content when stored at about 40° C. for a period of at least about 1 month (aspect 34).

In aspects, the invention provides the ophthalmologically suitable composition according to any one or more of aspects 1-34, wherein the composition retains at least 99% of the initial latanoprost compound composition content when stored at about 40° C. for a period of at least about 2 months (aspect 35).

In aspects, the invention provides the ophthalmologically suitable composition according to any one or more of aspects 1-35, wherein the composition retains at least 99% of the initial latanoprost compound composition content when stored at about 40° C. for a period of at least about 3 months (aspect 36).

In aspects, the invention provides the ophthalmologically suitable composition according to any one or more of aspects 1-36, wherein the composition retains at least 99% of the initial latanoprost compound composition content when stored at about 40° C. for a period of at least about 6 months (aspect 37).

In aspects, the invention provides the ophthalmologically suitable composition according to any one or more of aspects 1-37, wherein the composition has a pH of 6.5-about 7 (aspect 38).

In aspects, the invention provides the ophthalmologically suitable composition according to aspect 38, wherein the composition has a pH of 6.6-6.8 (aspect 39).

In aspects, the invention provides the ophthalmologically suitable composition according to any one or more of aspects 1-39, wherein the composition comprises less than 0.1% of any polyacrylic acid polymer (aspect 40).

In aspects, the invention provides the ophthalmologically suitable composition according to aspect 40, wherein the composition is free of any detectable amount of any polyacrylic acid polymer, such as any carbomer-type polymer (e.g., carbomer 934, carbomer 934 P, carbomer 941, Carbopol 910, and carboxyvinyl polymer) (aspect 41).

In aspects, the invention provides the ophthalmologically suitable composition according to any one or more of aspects 1-41, wherein the remainder of the composition is at least primarily composed of a phosphate buffer, sodium chloride, and water (aspect 42).

In aspects, the invention provides the ophthalmologically suitable composition according to aspect 42, wherein the remainder of the composition is essentially composed of a formulation that comprises sodium chloride in a concentration of about 0.1% to about 0.5%, monobasic or dibasic sodium phosphate in a concentration of between about 0.05 to 1.0%, and water (aspect 43).

In aspects, the invention provides the ophthalmologically suitable composition according to any one or more of aspects 1-43, wherein the latanoprost compound is the primary prostaglandin analog in the composition (aspect 44).

In aspects, the invention provides the ophthalmologically suitable composition according to aspect 44, wherein the latanoprost compound is essentially the only prostaglandin analog in the composition (aspect 45).

In aspects, the invention provides the ophthalmologically suitable composition according to any one or more of paragraphs aspects 1-45, wherein the latanoprost compound is the only ophthalmological active pharmaceutical ingredient in the composition (aspect 46).

In aspects, the invention provides the ophthalmologically suitable composition according to any one or more of aspects 1-45, wherein the latanoprost compound is the only active pharmaceutical ingredient in the composition (aspect 47).

In aspects, the invention provides the ophthalmologically suitable composition according to any one or more of aspects 1-46, wherein the composition comprises at least one additional active pharmaceutical ingredient (aspect 48).

In aspects, the invention provides the ophthalmologically suitable composition according to aspect 48, wherein the at least one additional active pharmaceutical ingredient comprises a non-latanoprost compound prostaglandin analog (aspect 49).

In aspects, the invention provides the ophthalmologically suitable composition according to aspect 49, wherein the non-latanoprost compound prostaglandin analog is selected from bimatoprost, travoprost, trimoprostil, rioprostil, cloprostenol, fluprostenol, luprostiol, etiproston, tiaprost, and unoprostone; an ophthalmologically suitable derivative of any thereof, an ophthalmologically suitable salt of any thereof, or a combination of any thereof (aspect 50).

In aspects, the invention provides the ophthalmologically suitable composition according to aspect 50, wherein the non-latanoprost compound prostaglandin analog comprises an isopropyl, misoprostol, sulfoprostone, gemeprost, alfaprostol, or delprostenate derivative of a prostaglandin analog, such as unoprostone (aspect 51).

In aspects, the invention provides the ophthalmologically suitable composition according to any one or more of aspects 48-51, wherein the at least one additional active pharmaceutical ingredient comprises a non-prostaglandin analog antiglaucoma agent (aspect 52).

In aspects, the invention provides the ophthalmologically suitable composition according to aspect 52, wherein the non-prostaglandin analog antiglaucoma agent is a beta blocker, carbonic anhydrase inhibitor, alpha-adrenergic agonist, or a combination of two or more of any thereof, or a combination of any one or more of any two or more types of such agents (aspect 53).

In aspects, the invention provides the ophthalmologically suitable composition according to aspect 52, wherein the non-prostaglandin analog antiglaucoma agent is a rho kinase inhibitor, such as netarsudil (aspect 54).

In aspects, the invention provides the ophthalmologically suitable composition according to any one or more of aspects 48-54, wherein the composition is essentially free or free of alcohol(s) (e.g., except for any of the alcohols described herein, free of polyvinyl alcohol, or free of any alcohol not explicitly recited), castor oil, potassium sorbate, travoprost, boric acid, disodium EDTA, timolol, glycerin, propylene glycol, sorbic acid, chlorobutanol, HPMC (hydroxypropyl methylcellulose), hydroxy propyl cellulose, polyvinylpyrrolidone, tartaric acid, tyloxapol, trometamol, bimatoprost, diclofenac, flurbiprofen, ketorolac, hydroxystearate compositions (e.g., hydroxystearate 40), macrogolglycerol compounds (e.g., macrogolglycerol hydroxystearate, such as macrogolglycerol hydroxystearate 40), aminocaproic acid (e.g., ε-aminocaproic acid), any cyclodextrin, trehalose, isopropanol, sodium citrate, non-PCCC-associated PEGs (e.g., large free PEGs (e.g., PEG 400 or larger, such as PEG 1000 or larger)), sorbitol, mannitol, polyethylene glycol hydroxy stearate, macrogol compounds (e.g., macrogol 4000), disodium edetate, sodium hydroxide, nepafenac, sodium dihydrogen phosphate monohydrate, disodium hydrogen phosphate anhydrous, propylene glycol, or a combination of any, any several, or all thereof (aspect 55).

In aspects, the invention provides the ophthalmologically suitable composition according to aspect 55, wherein the composition is free of castor oil, potassium sorbate, boric acid, disodium EDTA (ethylenediaminetetraacetic acid), tyloxapol, trometamol, macrogolglycerol compounds (e.g., macrogolglycerol hydroxystearate, such as macrogolglycerol hydroxystearate 40), sorbitol, macrogol compounds (e.g., macrogol 4000), disodium edetate, mannitol, sodium hydroxide, nepafenac, sodium dihydrogen phosphate monohydrate, disodium hydrogen phosphate anhydrous, propylene glycol, or a combination of any, several, or all thereof (aspect 56).

In aspects, the invention provides the ophthalmologically suitable composition according to any one or more of aspects 1-56, wherein the composition has a readable viscosity of about 1 to about 1.5 (using the method described in Kolo and Jensen, 2017) (aspect 57).

In aspects, the invention provides the ophthalmologically suitable composition according to any one or more of aspects 1-57, wherein the composition is stored in a packaging that facilitates the delivery of the composition as eye drops (aspect 57).

In aspects, the invention provides the ophthalmologically suitable composition according to aspect 57, wherein the container significantly reduces exposure of the latanoprost compound to UV B radiation, such as by at least about 50%, at least about 65%, at least about 75%, at least about 90%, at least about 95%, or at least 99% (aspect 59).

In aspects, the invention provides the ophthalmologically suitable composition according to any one or more of aspects 1-59, wherein the composition contains less than 2.5% of impurities, such as less than 2%, less than 1.75%, less than 1.5%, less than 1.25%, or less than 1% impurities (aspect 60).

In aspects, the invention provides the ophthalmologically suitable composition according to any one or more of aspects 1-60 wherein the latanoprost compound(s) primarily comprise, ≥˜75% comprise, ≥˜90% comprise, consist essentially of, or consist of latanoprost (aspect 61).

In aspects, the invention provides a method for modulating the physiological conditions of the eye in a mammalian subject comprising administering a therapeutically effective amount of an ophthalmologically suitable composition according to any one or more of aspects 1-61 (aspect 62).

In aspects, the invention provides the method of aspect 62 wherein the method is performed under conditions that treat open-angle glaucoma, reduce intraocular pressure, or both (aspect 63).

In aspects, the invention provides the method of aspect 63, wherein the subject is a human patient diagnosed with one or both conditions (aspect 64).

In aspects, the invention provides the method according to any one or more of aspects 62-64, wherein the method comprises administering one drop of an effective amount of the ophthalmologically suitable composition from a container adapted to administer eye drops per day (aspect 65).

In aspects, the invention provides the method of aspect 65 wherein each drop contains about 1.5 μg of one or more latanoprost compounds (aspect 66).

In aspects, the invention provides the method of aspect 65 or aspect 66, wherein the average volume of each drop of the ophthalmologically suitable composition is about 40 μL to about 50 μL (aspect 67).

In aspects, the invention provides the method according to any one or more of aspects 65-67, wherein the method comprises shipping the eye drop containers at temperatures above 15° C. (aspect 68).

In aspects, the invention provides the method according to any one or more of aspects 62-68, wherein the method comprises administering the product after the product has been stored at a temperature of greater than about 35° C. or 40° C. for a period of more than 10 days, such as more than 2 weeks, or more than about 1 month (aspect 69).

In aspects, the invention provides the method according to any one or more of aspects 62-69, wherein the method comprises continuing to administer the ophthalmologically suitable composition after the container has been opened and stored at temperature of greater than about 20° C. for period of about 8 weeks or longer (aspect 70).

In aspects, the invention provides the method of according to any one or more of aspects 62-70, wherein the method comprises administering the ophthalmologically suitable composition to the subject for a period of at least six months with an average decrease in intraocular pressure (IOP) of at least 15% throughout or at the end of the treatment period with a significant number of subjects exhibit decrease of IOP of at least 20%, 25%, or 30%, throughout or at the end of the treatment period (aspect 71).

In aspects, the invention provides the method of according to any one or more of aspects 62-71 wherein the method comprises administering a second antiglaucoma agent to the subject (aspect 72).

In aspects, the invention provides the method of aspect 72, wherein the second antiglaucoma agent is administered with the latanoprost compound as a combination composition (aspect 73).

In aspects, the invention provides the method of aspect 72, wherein the second antiglaucoma agent is administered separately from the ophthalmologically suitable composition and the timing of administration of the ophthalmologically suitable composition and the second antiglaucoma agent is separated by at least about 5 minutes (aspect 74).

In aspects, the invention provides the use of a composition according to any one or more of aspects 1-61 or incorporating any of the conditions of the methods of aspects 62-74 in the preparation of a medicament for treating open-angle glaucoma, reducing intraocular pressure, or both (aspect 75).

In aspects, the invention provides an ophthalmic composition comprising latanoprost in an amount of about 0.001 to about 0.01% by weight, polyoxyl 40 hydrogenated castor oil in an amount of about in an amount of about 0.1% to about 5.0% by weight and one or more pharmaceutically acceptable excipient, wherein the composition remains stable when stored at room temperature conditions and/or accelerated conditions for at least 3 months (aspect 76).

In aspects, the invention provides the ophthalmic composition of aspect 76, wherein the amount of latanoprost is 0.005% (aspect 77).

In aspects, the invention provides the ophthalmic composition of aspect 76 or aspect 77, wherein the amount of polyoxyl 40 hydrogenated castor oil is about 1.0% by weight (aspect 78).

In aspects, the invention provides the ophthalmic composition of any one or more of aspects 76-78, wherein the bimatoprost, travoprost, trimoprostil, rioprostil, cloprostenol, fluprostenol, luprostiol, etiproston, tiaprost, unoprostone and its derivatives like unoprostone isopropyl, misoprostol, sulfoprostone, gemeprost, alfaprostol, delprostenate, and the like is used as an alternative to latanoprost, alone or in combination (aspect 79).

In aspects, the invention provides the ophthalmic composition of any one or more of aspects 76-79, wherein the composition in the form of solutions, suspensions, ointments, gels, emulsions, oils, and other dosage forms for topical administration (aspect 80).

In aspects, the invention provides the ophthalmic composition of any one or more of aspects 76-80, wherein the composition is distributed into single dose or multidose containers (aspect 81).

In aspects, the invention provides the ophthalmic composition of any one or more of aspects 76-81, wherein the composition having a pH of about 6.5-7 or about 6.5-6.9, such as about 6.7 (aspect 82).

In aspects, the invention provides the ophthalmic composition of any one or more of aspects 76-82, wherein the composition is free of any gelling agent of the Carbomer type (e.g., a cross-linked polymer, a polymer having a molecular weight of greater than 10,000, or any other gelling agent) (the lack of a gelling agent can be applied to other aspects described above, as well) (aspect 83).

In aspects, the invention provides the ophthalmic composition of any one or more of aspects 76-83, wherein the polyoxyl-40 hydrogenated castor in the better stabilization effect compared to polysorbate-80 in the formulation (aspect 84).

In aspects, the invention provides the composition of any one or more of aspects 76-84, wherein the one or more pharmaceutically acceptable excipient comprises solubilizing agent, thickening agent or viscosity-enhancer, chelating agent, tonicity agents, preservatives, surfactants, buffering agents, pH-adjusting agents, and water (aspect 85).

In aspects, the invention provides the ophthalmic composition of aspect 85, wherein ionic tonicity agent(s) is sodium chloride (aspect 86).

In aspects, the invention provides the ophthalmic composition of aspect 85 or aspect 86, wherein preservative (s) is benzalkonium chloride (aspect 87).

In aspects, the invention provides the ophthalmic composition of any one or more of aspects 85-87, wherein buffer(s) is phosphate buffer (aspect 88).

In aspects, the invention provides the ophthalmic composition of any one of aspects 76-88, wherein the composition is a solution and applied once or twice in the affected eye(s) for treating or preventing glaucoma (aspect 89).

In aspects, the invention provides the ophthalmic composition of any one of aspects 85-89, wherein the composition comprises about 0.005% of latanoprost, about 1.0% of polyoxyl 40 hydrogenated castor oil, phosphate buffer, benzalkonium chloride, sodium chloride, and water (aspect 90).

In aspects, the invention provides the ophthalmic composition of any one of aspects 76-90, wherein the composition further comprising second antiglaucoma agent selected from beta blockers, carbonic anhydrase inhibitors or alpha-adrenergic agonists (aspect 91).

In aspects, the invention provides an ophthalmic composition comprising: latanoprost in an amount of about 0.001% to about 0.01% by weight, polyoxyl 40 hydrogenated castor oil in an amount of about 0.1% to about 5.0% by weight, benzalkonium chloride in an amount of about 0.0% to about 0.02% by weight, sodium chloride in a concentration of about 0.1% to about 0.5%, monobasic and or dibasic sodium phosphate in a concentration of between about 0.05 to 1.0% and water, wherein the composition remains stable when stored at room temperature conditions and/or accelerated conditions for at least 3 months (aspect 92).

In aspects, the invention provides a method for treating open-angle glaucoma or reducing intraocular pressure comprising: latanoprost in an amount of about 0.001% to about 0.01% by weight, polyoxyl 40 hydrogenated castor oil in an amount of about 0.1% to about 5.0% by weight, benzalkonium chloride in an amount of about 0.0% to about 0.02% by weight, sodium chloride in a concentration of about 0.1% to about 0.5%, monobasic and or dibasic sodium phosphate in a concentration of between about 0.05 to 1.0% and water, wherein the composition remains stable when stored at room temperature conditions and/or accelerated conditions for at least 3 months (aspect 93).

In aspects, the invention provides a composition that incorporates any combination of the preceding composition aspects of this Exemplary Aspects Section (aspect 94).

In aspects, the invention provides a composition according to aspect 94 that incorporates any approximate amount of any of the excipients and active ingredient listed in the Examples below (aspect 95).

In aspects, the invention provides a method that incorporates any two or more method aspects of this Exemplary Aspects Section (aspect 96).

In aspects, the invention provides the method of any one or more of aspects 62-74, wherein the application of the method results in a 0.5-hour post administration to 24-hour post administration mean percent reduction in intraocular pressure which is at least statistically similar to or greater than that achieved by treating a similarly increased intraocular pressure in an at least similar mammalian subject for the same period of time with the composition approved by the United States Food and Drug Administration under NDA number 020597 (aspect 97).

In aspects, the invention provides the method of any one or more of aspects 62-74, wherein the application of the method results in a 0.5-hour post administration to 24-hour post administration mean intraocular pressure which is at least statistically similar to that achieved by that achieved by treating a similarly increased intraocular pressure in an at least similar mammalian subject for the same period of time with the composition approved by the United States Food and Drug Administration under NDA number 020597 (aspect 98).

In aspects, the invention provides a composition of any one or more of aspects 1-92, aspects 93-94, or aspect 95, wherein the composition is stored in a container sterilized by ethylene oxide sterilization (aspect 98).

In aspects, the invention provides a composition of any one or more of aspects 1-92, aspects 93-94, or aspect 95, wherein the composition is stored in a container sterilized by gamma sterilization.

In aspects, the invention provides a composition of any one or more of aspects 1-92, aspects 93-94, or aspect 95, wherein the composition is stored in a container sterilized by ethylene oxide.

In aspects, the invention provides a composition of any one or more of aspects 1-92, aspects 93-94, or aspect 95, wherein the composition demonstrates a detectably or significantly better stability when stored in a container sterilized by gamma sterilization compared to the same composition stored in a container sterilized by ethylene oxide sterilization, as indicated by one or more measure of stability such as, e.g., an assay of latanoprost (e.g., an indication of loss of or prevention of loss of latanoprost compound(s)), an assay of related latanoprost compounds, e.g., one or more compounds related to latanoprost, a measure of one or more impurities, measurement of pH, measurement of osmolality, or any combination thereof, or, e.g., by other methods of stability known in the art.

EXAMPLES

It is to be understood that the above description is intended to be illustrative and not restrictive. Many embodiments will be apparent to those of skill in the art upon reading the above description. The scope of the invention should, therefore, be determined with reference to the claims of the invention, along with the full scope of equivalents to which such claims are entitled.

### Example I

Composition with Polyoxyl 40 Hydrogenated Castor Oil (Cremophor® RH40)

TABLE 1
Exemplary composition 1, P0047-3-3
(PCCC = Cremophor ® RH40).

| Sr. | | 1 mL | |
|---|---|---|---|
| No | Ingredient | Total Volume | |
| 1 | Latanoprost | 0.05 | mg |
| 2 | Benzalkonium chloride | 0.20 | mg |
| 3 | Sodium chloride | 4.10 | mg |
| 4 | Monobasic sodium phosphate USP | 4.60 | mg |
| | monohydrate | | |
| 5 | Dibasic sodium phosphate USP | 4.74 | mg |
| | anhydrous | | |
| 6 | Polyoxyl 40 hydrogenated castor oil | 10.0 | mg |
| | (Cremophor RH40) | | |
| 7 | Water for Injection q.s. to | 1 | mL |

### Manufacturing Process: A Manufacturing Process of the Above Exemplary Composition (Table 1) has the Following Steps:

1. In a suitable container, take the required amount of Polyoxyl 40 hydrogenated castor oil and add 25% of batch weight of water for injection to it. Initiate stirring.
2. Add the required amount of benzalkonium chloride and ensure thorough dissolution of the mixture.
3. Stir the contents slowly to avoid potential foaming
4. To the above solution, add the required amount of latanoprost and ensure its dissolution.
5. Once latanoprost is dissolved, add WFI so that the batch weight is about 80% of the target.
6. Add the remaining ingredients, such as the two phosphates and sodium chloride, one after the other, making sure that the previous ingredient is in solution prior to the addition of the next.
7. Quantity sufficient (q.s.) the solution to 100%.
8. Filter the solution through suitable filter and transfer it to a suitable sterile tank.
9. Package the product aseptically in previously sterilized ophthalmic containers. Insert the tip and cap the product.
10. Inspect the bottles for extraneous particles and label the bottles.

Formulations prepared according to the invention as described in Example 1 were tested for stability under refrigerated conditions ((2-8° C.), typically (25° C./60% relative humidity) and accelerated conditions (40° C./75% relative humidity) to determine stability of such formulations. The stability data of the composition provided in Table 1 of Example 1 was also compared with Reference Product (XALATAN®). Stability results are provided in Table 3.

### Example 2

### Comparative Composition (Polysorbate-80)

TABLE 2
Exemplary composition 2, P0047-3-2
(Comprising Polysorbate-80)

| Sr. | | 1 mL |
|---|---|---|
| No | Ingredient | Total Volume |
| 1 | Latanoprost | 0.05 mg |
| 2 | Benzalkonium chloride | 0.20 mg |
| 3 | Sodium chloride | 4.10 mg |
| 4 | Monobasic sodium phosphate USP | 4.60 mg |
| | monohydrate | |
| 5 | Dibasic sodium phosphate USP anhydrous | 4.74 mg |
| 6 | Polysorbate-80 | 10.0 mg |
| 7 | Water for Injection | q.s. to 1 mL |

The composition of Table 2 was prepared according to manufacturing process of Example 1.

The stability data for the composition of Table 1, Example 1 (comprising the PCCC polyoxyl 40 hydrogenated castor oil), was compared to the composition of Table 2, Example 2, comprising the polysorbate-80. Stability results are provided in Table 3.

TABLE 3
Comparative stability data.
(Reference Listed Drug (RLD) composition
(XALATAN ®) vs. test composition comprising
Polysorbate 80 vs. test composition
with PCCC = Cremophor ® RH40)

| | | P0047- | P0047-3- | |
|---|---|---|---|---|
| | | 1-3 | 3 (1% | |
| | | (RLD | Cremophor ® | P0047-3-2 |
| Storage | | compo- | RH40, | (1% PS-80, |
| conditions | Time | sition) | Example 1) | Example 2) |
| 40° C. | Initial | 92.6 | 99.5 | 101 |
| | 1 week | 88.4 | 100.1 | 98.5 |
| | 1 month | 76 | 99.6 | 96.3 |
| | 2 months | 74.5 | 99.3 | 87.5 |
| | 3 months | 71.6 | 94.2 | 77.7 |
| | 6 months | — | 89.4 | 64.0 |
| 25° C. | Initial | 92.6 | 99.5 | 101 |
| | 1 month | 89.7 | 98.6 | 99.3 |
| | 2 months | 89.2 | 100.3 | 98.9 |
| | 3 months | 89.7 | 100.2 | 98.3 |
| | 6 months | — | 101.4 | 88.7 |
| 2-8° C. | Initial | 92.6 | 99.5 | 101 |
| | 2 months | 91.6 | 98.2 | 100.4 |
| | 3 months | 91.6 | 99.9 | 99.8 |
| | 6 months | — | 100.6 | 100.4 |

Despite the similar hydrophilic-lipophilic balance (HLB) values of polysorbate-80 and polyoxyl 40 hydrogenated castor oil (HLB values of polysorbate-80 and hydrogenated castor oil (Cremophor® RH40/Kolliphor® RH 40) being 15 and 14-16 respectively), the above stability results demonstrate that Polysorbate-80 and Cremophor® RH 40 vary in their ability to maintain composition stability. In the latanoprost formulations tested, the polysorbate 80 had a marginal effect on stabilization, while the PCCC Cremophor® RH 40 demonstrated strong efficacy. Based on the 6 months of data analyzed, it has been demonstrated that the Cremophor® RH 40 formulation has some degradation of latanoprost in accelerated stability testing conditions, however no degradation was observed under room temperature conditions. Based on these results, it is expected that the tested composition comprising latanoprost and Cremophor® RH 40 would likely be capable of maintaining stability (e.g., be stable, such as maintain physical and chemical stability) over the course of 24 months. Further, the results demonstrate that the exemplified Cremophor® RH 40 formulation outperformed the RLD product in stability testing.

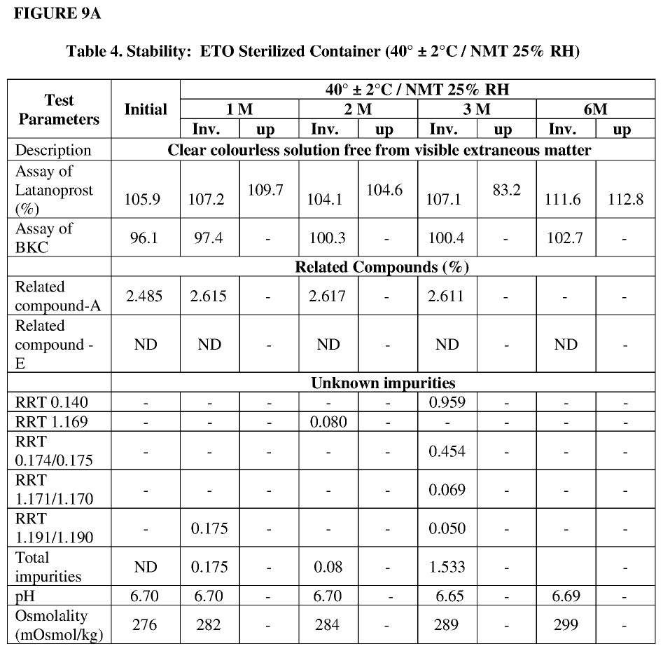

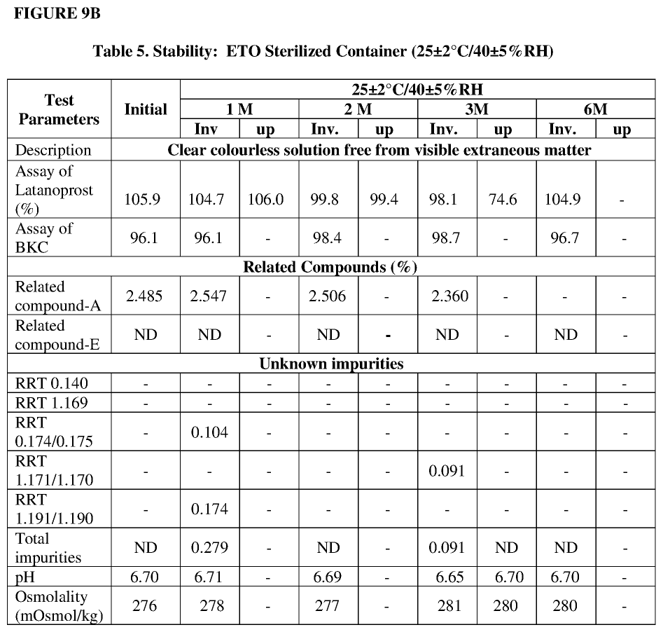

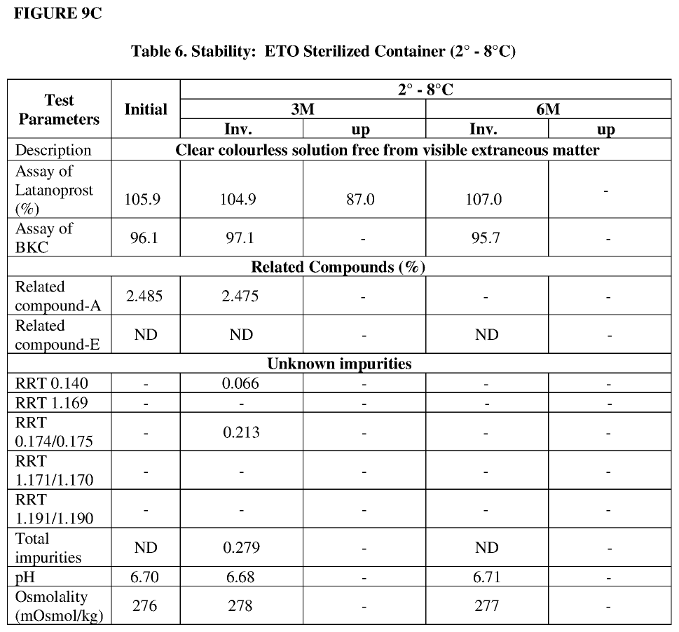

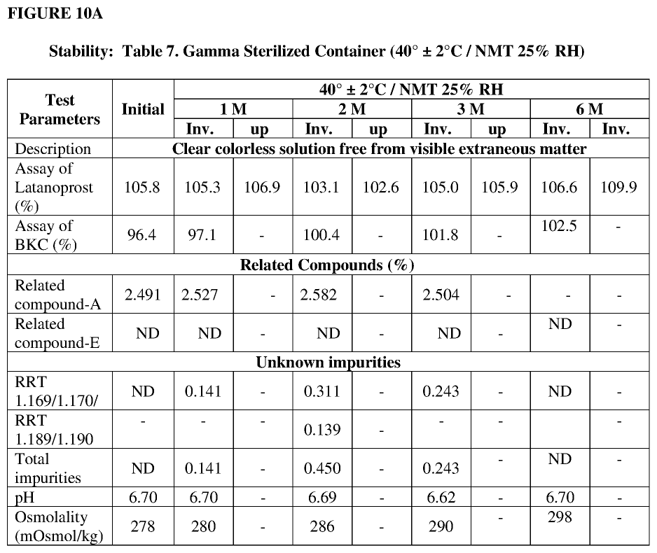

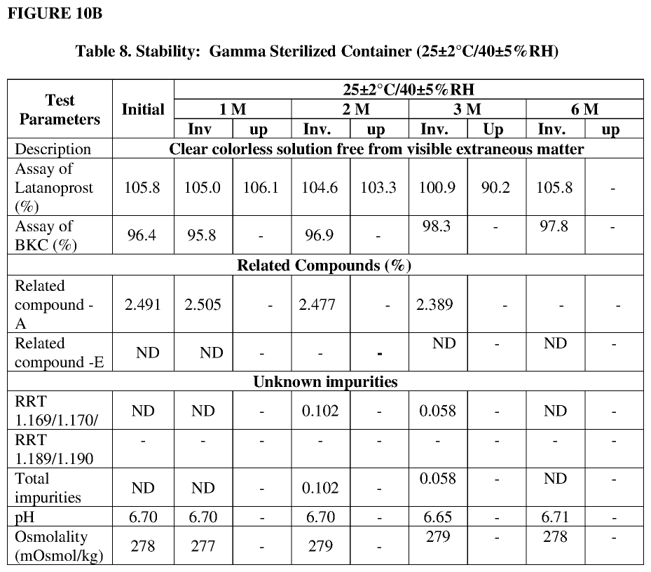

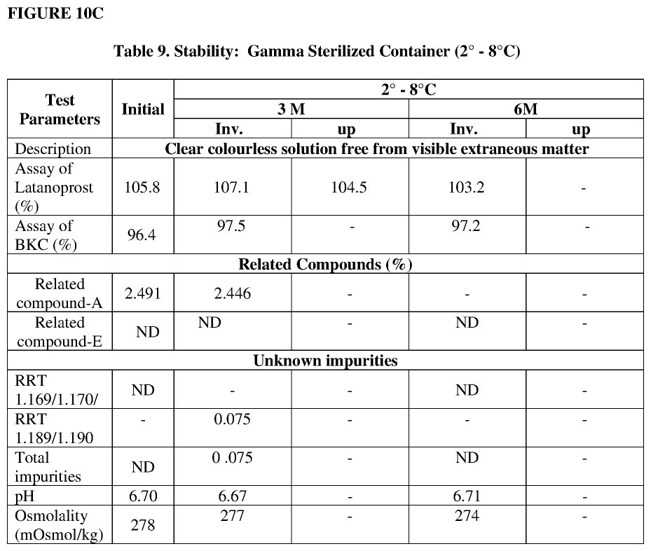

As a further component of Example 2, the composition of Example 1 (P0047-3-3 (1% Cremophor® RH40)) was filled in both ethylene oxide (ETO) and gamma sterilized containers to examine effect of sterilization method of such containers on composition stability. Stability testing was performed according to common practices known and accepted in the art, e.g., testing compositions in containers stored upright (“up”) and inverted (“inv”). Compositions filled in containers sterilized by both methods were subjected to stability testing. Stability testing results are summarized in FIGS. 9A-C (Tables 4-6) & FIGS. 10A-C (Tables 7-9).

FIGS. 9A-9C provide stability data for the P0047-3-3 (1% Cremophor® RH40) composition when stored in ethylene oxide-sterilized containers. FIG. 9A (Table 4) provides stability data for the composition stored at 40°±2° C./NMT 25% RH in ethylene oxide-sterilized containers. FIG. 9B (Table 5) provides stability data for the composition stored at 25±2° C./40±5% RH in ethylene oxide-sterilized containers. FIG. 9C (Table 6) provides stability data for the composition stored at 2°-8° C. in ethylene oxide-sterilized containers.

FIGS. 10A-10C provide the stability data for the same P0047-3-3 (1% Cremophor® RH40) composition when stored in gamma-sterilized containers. FIG. 10A (Table 7) provides stability data for the composition stored at 40°±2° C./NMT 25% RH in gamma-sterilized containers. FIG. 10B (Table 8) provides stability data for the composition stored at 25±2° C./40±5% RH in gamma-sterilized containers. Finally, FIG. 10C (Table 9) provides stability data for the composition stored in gamma-sterilized containers.

The comparative results of the stability testing infer that products filled in gamma sterilized containers are slightly better for stability than ETO sterilized containers, particularity in terms of latanoprost assay and unknown impurities.

### Example 3

Table 4 below provides a listing of exemplary ingredients suitable for an exemplary formulation of the ophthalmic composition of the present invention.

TABLE 4
Exemplary composition 3.

| Sr. | | 1 mL | |
|---|---|---|---|
| No | Name of ingredients | Total Volume | |
| 1 | Latanoprost | 0.05 | mg |
| 2 | Benzalkonium chloride | 0.005 mg to 0.2 mg | |
| 3 | Sodium chloride | 4.10 | mg |
| 4 | Monobasic sodium phosphate USP | 4.60 | mg |
| | monohydrate | | |
| 5 | Dibasic sodium phosphate USP anhydrous | 4.74 | mg |
| 6 | Polyoxyl 40 hydrogenated castor oil | 1 mg to 20 mg | |
| | (Cremophor RH40) | | |
| 7 | Milli-Q water q.s. to | 1 | mL |

### Example 4

### Animal Study

An animal study was conducted to evaluate the efficacy of reference formulation Xalatan® 0.005% (“Test Item M”) and test formulation P0047-3-3 (1% Cremophor® RH40) (“Text Item N”) in reducing corticosteroid induced intraocular pressure in a rat model, the use of a rat model for ocular pressure (e.g., in glaucoma research) being an accepted animal model in the art (see, for example the review article by Evangelho, et. al., “Experimental Models of Glaucoma: a Powerful Translational Tool for the Future Development of New Therapies for Glaucoma in Humans—A Review of the Literature”, in Medicina, published 17 Jun. 2019.)

The purpose of the intraocular pressure (IOP) animal study was to demonstrate that formulations having the characteristics of formulations described herein can possess comparable IOP-lowering results to the well-established Xalatan® 0.005% on-market ophthalmic product. Specifically, Test Item N, Formulation P0047-3-3 (comprising 1% Cremophor® RH40, is a formulation comprising the same amount of latanoprost as Xalatan® 0.005%, but additionally contains 1% cremophor.

### Study Design

Male Sprague Dawley rats at approximately 14 weeks of age, after being clinically examined for good health and found suitable for use in the experiment according to standards known in the art, were appropriately acclimated and used in this study. The study was conducted under controlled conditions and according to standard experimental animal care guidelines, regulations, and practice.

All rats were housed in a single, environment-controlled room at 16-23° C. and a relative humidity of 57-67% during the experimental period. The photoperiod was 12 hours light, 12 hours darkness. Adequate fresh air supply of about 12.6 air changes/hour was maintained in the experimental room. Maximum and minimum temperatures and relative humidity in the experimental room was monitored and recorded once daily Animals were housed 2 per cage in standard animal cages with facilities for pelleted food and drinking water in a polycarbonate bottle with stainless steel sipper tubes. Pelleted, standard rodent diet food, and filtered, purified drinking water, were provided ad libitum. Animal cages and water bottles were changed at least once per week over the course of the study. Animals were acclimatized for 5 days after a quarantine period in a single study room.

### Induction Phase

Post-acclimatization, the phenotype development phase was initiated. Ocular hypertension (increased intraocular pressure (IOP)) was induced in animals by the topical application of an ophthalmic dexamethasone formulation (“Dex”) comprising 0.1% dexamethasone in 0.9% saline to both the left and right eye of all rats. The 0.1% dexamethasone in 0.9% saline solution (Dex) was prepared fresh each day and 40 μL of the Dex composition was applied to each eye. A saline control group was also established, applying 40 μL of saline to both eyes of rats in the saline control group. Dex instillation was initiated in a batch wise fashion with 28 animals in each batch; 3 saline control animals were included in each batch. Three batches were initiated, on consecutive days, with a one day gap between each batch. The Dex installation group comprised a total of 84 animals while the saline control group comprised 9 animals in total.

Dex and saline applications were performed four time (4×) per day with an approximately 2-hour gap between each instillation. Instillation was carried out for a duration of 5 weeks. During the induction phase, intraocular pressure was measured by tonometer (calibrated Tono-Pen-AVIA, AMETEK, Inc. Reichert Technologies) once weekly, as the end of each week. Dexamethasone topical instillation into eyes, four times per day and for a period of five weeks significantly increased intraocular pressure compared to saline treated normal animals, indicating development of the target phenotype in the animal model.

At the end of the induction phase (5 weeks), Dex group animals were randomized based on IOP readings. 12 animals were selected from each of the three batches. From each group of 12 animals selected per batch, 6 animals were treated with Test Item M (Xalatan® 0.005%) and 6 animals were treated with Test Item N (P0047-3-3 (1% Cremophor® RH40); see composition in Table 1, Example 1, above). Further, from each batch after completion of saline instillation, 2 animals were selected for study based on IOP recording. 6 total animals were selected for saline treatment Animals were randomized for treatment groups (Test Item M, Xalatan® 0.005%, group and Test Item N, P0047-3-3 (1% Cremophor® RH40)), group based on intraocular pressure readings.

Basal mean IOP difference between Test Item M and Test Item N groups were observed as being ˜6% at the commencement of treatment. IOP mean range was observed as being between 24.85 and 26.12 at the time of treatment initiation. The test animals were 19 weeks of age at the start of treatment.

### Treatment and IOP Measurement

Single topical instillation treatment was carried out in a staggered manner as per batch which completed the 5-week induction phase. Selected animals from the batches were treated with either 40 μL Xalatan® 0.005% (Test Item M) or 40 μL P0047-3-3 (1% Cremophor® RH40) (Test Item N), each instilled into the right eye of each animal for each respective treatment group. 40 μL saline was instilled into the left eye of each same animal.

### Observations

Intraocular pressure was recorded at each of the following time points post treatment: 0.5 h, 1 h, 2 h, 3 h, 4 h, 6 h, 8 h, 12 h, 18 h, and 24 h. Miosis and conjunctival hyperemia was observed post intraocular pressure recordings at each of the following time points post-treatment: 1 h, 3 h, 6 h, 12 h, 18 h and 24 h.

### Results

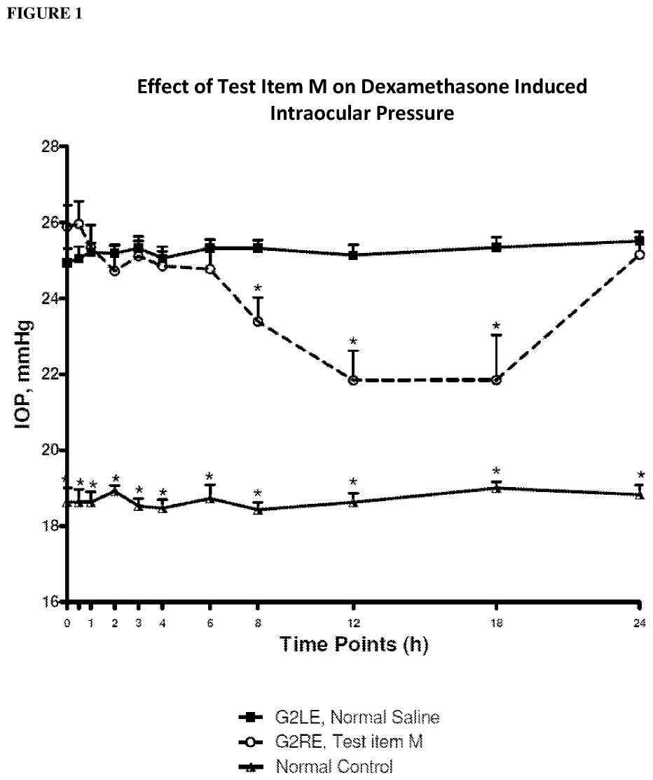

All measured and presented values were expressed as mean±SEM. Significance (p<0.05) as compared to vehicle (saline) was determined by unpaired t-test using Graph pad Prism version 6. Significant outliers were determined using Grubb's outlier test. Such identified outliers were not considered in calculations. Results of the study are shown in FIGS. 1-8.

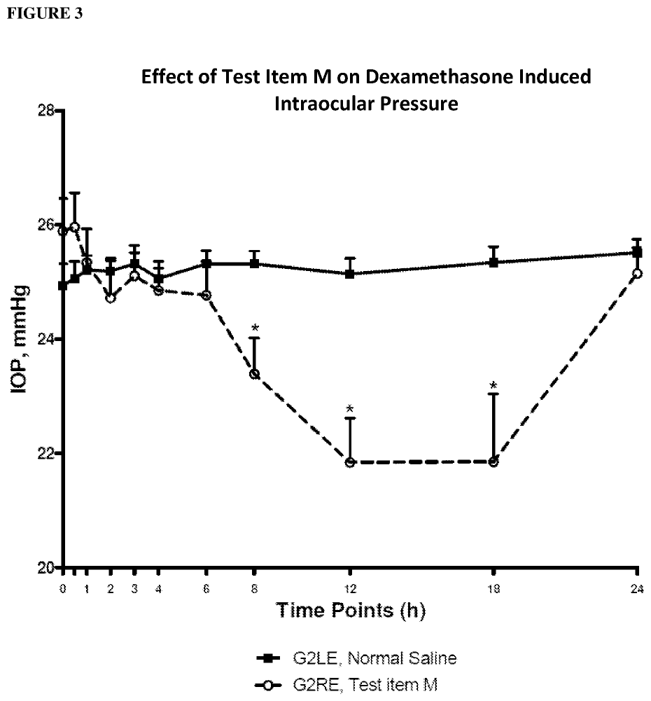

FIG. 1 and FIG. 3 illustrate the effect of a single instillation of Xalatan® 0.005% (Test Item M) on intraocular pressure in the steroid-induced ocular hypertension rat model study. The graphs represent the average IOP reading at each time point (0-24 hours). As described above, data are presented as mean±SEM; n=12 saline control and n=18 Test Item M (Xalatan® 0.005%), and Normal Saline; and *p<0.05 versus vehicle compared with unpaired t-test. As shown, Xalatan® 0.005% (Test Item M) showed a significant decrease compared to control saline at 8 h, 12 and 18 h post treatment time points.

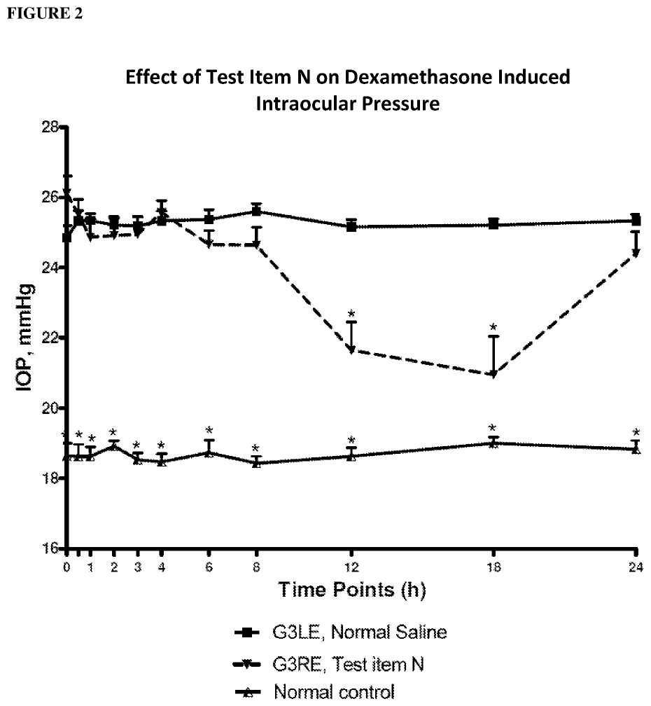

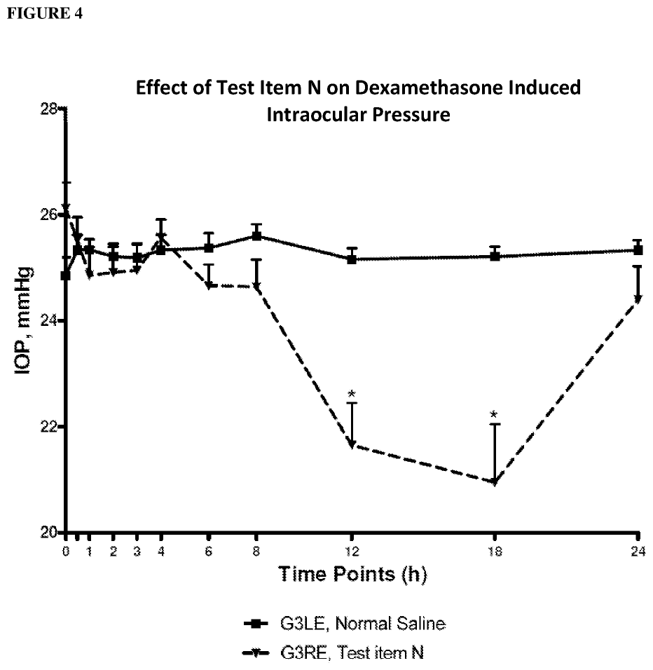

FIG. 2 and FIG. 4 show the effect of a single instillation of P0047-3-3 Formulation (1% Cremophor® RH40) (Test Item N) on intraocular pressure in the steroid-induced ocular hypertension rat model study. The graphs represent the average IOP reading at each time point (0-24 hours) and the same statistical presentation applies (wherein here n=18 Test Item N (P0047-3-3 (1% Cremophor® RH40)), and Normal Saline). Much like Xalatan® 0.005% (Test Item M), the P0047-3-3 (1% Cremophor® RH40) formulation (Test Item N) showed significant decrease in intraocular pressure compared to saline at 12 h and 18 h post treatment time points. As shown, Test Item N demonstrates a detectable or significant decrease in IOP (mmHG) at about the 6 hours, 8 hour, 12 hour, 18 hour, and 24 hour time points compared to normal saline, e.g., demonstrating a decrease in IOP which is between about 1 mmHg to about 10 mmHg, such as, e.g., ˜1 mmHg-˜8 mmHg, ˜1 mmHg-˜6 mmHg, or ˜1-˜4 mmHg, ˜2 mmHg-˜10 mmHg, ˜4-˜10 mmHg, or, e.g., ˜6-˜10 mmHg at one or more such time points. In aspects, Test Item N demonstrates a reduction in IOP of between about 10%-about 30% at between about 12-18 hours, such as, e.g., between ˜15%-about 25% at between about 12-18 hours. In aspects, the reduction in IOP is detectably or significantly greater at one or more of the 8 hours, 12 hour, 18 hour, or 24 hour time points after administration. In aspects, such results can be applied to any one or more compositions (e.g., any one or more embodiments of compositions, e.g., aspects of the invention) described herein.

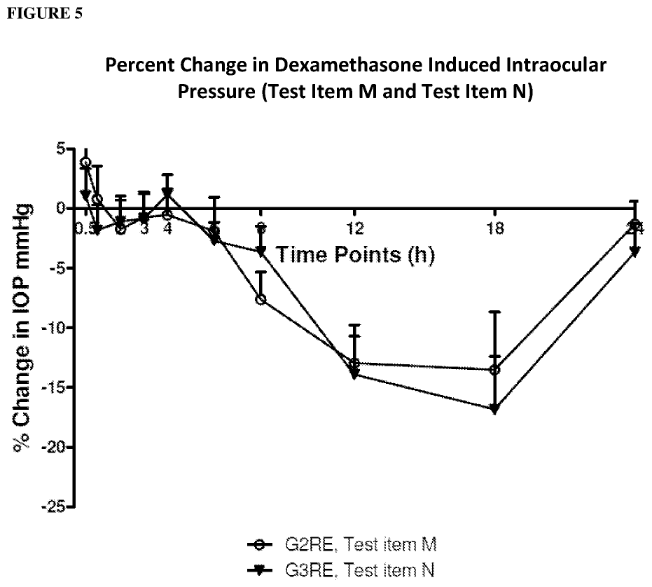

FIG. 5 shows the percent change in dexamethasone induced intraocular pressure demonstrated by a single administration of Test Item M (Xalatan® 0.005%) and Test Item N (P0047-3-3 (1% Cremophor® RH40)). The graph represents the average percent change at each time point (0-24 hours). Data is presented as Mean±SEM; n=18 Test Item M (Xalatan® 0.005%) and Test Item N (P0047-3-3 (1% Cremophor® RH40)). No statistical significance between Test Item M (Xalatan® 0.005%) and Test Item N (P0047-3-3 (1% Cremophor® RH40)) was observed at any of the time points through the 24-hour time point measured.

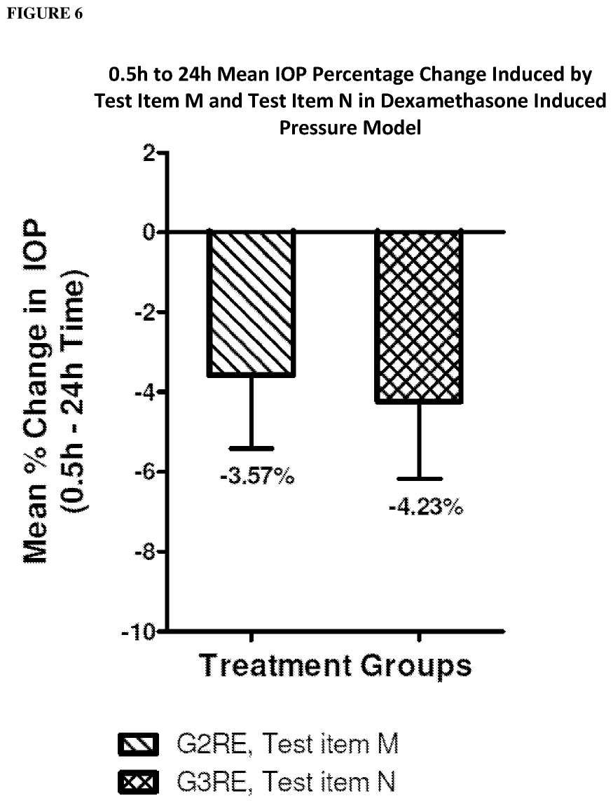

FIG. 6 shows the 0.5 to 24-hour mean percentage intraocular pressure change induced by Text Item M (Xalatan® 0.005%) and Test Item N (P0047-3-3 (1% Cremophor® RH40)) in the dexamethasone induced pressure model. Data is presented as Mean±SEM; n=18 Test Item M (Xalatan® 0.005%) and Test Item N (P0047-3-3 (1% Cremophor® RH40)). No statistically significant difference in 0.5 to 24 hour mean percent change in IOP was observed between Test Item M (Xalatan® 0.005%) and Test Item N (P0047-3-3 (1% Cremophor® RH40)). In aspects, Test Item N demonstrates a mean change in IOP which is between about 1%-about 10%, e.g., ˜2%-˜10%, ˜2%-8%, ˜2%-˜6%, or ˜2%-˜5%, such as ˜1%-˜8%, ˜1%-˜6%, or ˜1%-˜5%. In aspects, Test Item N demonstrates a mean change in IOP which is detectably or significantly greater than the mean change in IOP demonstrated by Test Item M.

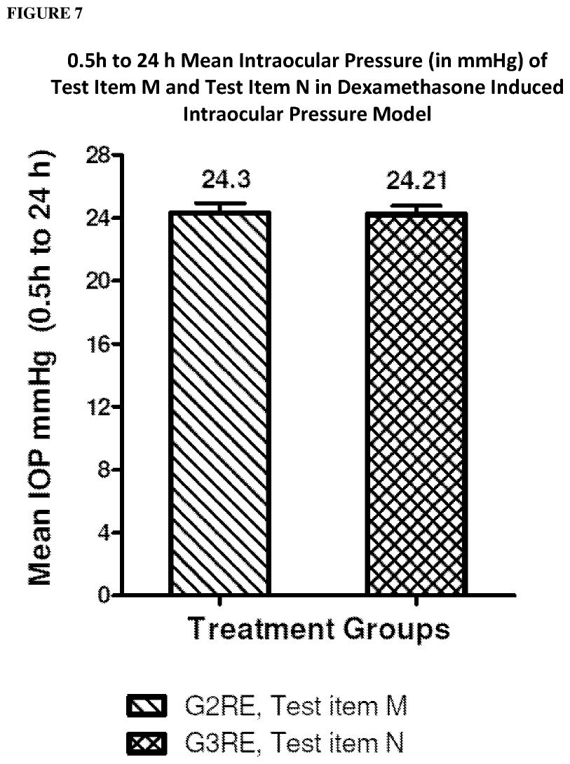

FIG. 7 shows the 0.5 to 24-hour mean intraocular pressure (mmHg) of Test Item M (Xalatan® 0.005%) and Test Item N (P0047-3-3 (1% Cremophor® RH40)) in the dexamethasone induced pressure model. Data is presented as Mean±SEM; n=18. No statistical significance in mean 0.5 to 24 hour IOP was observed between Test Item M and Test Item N.

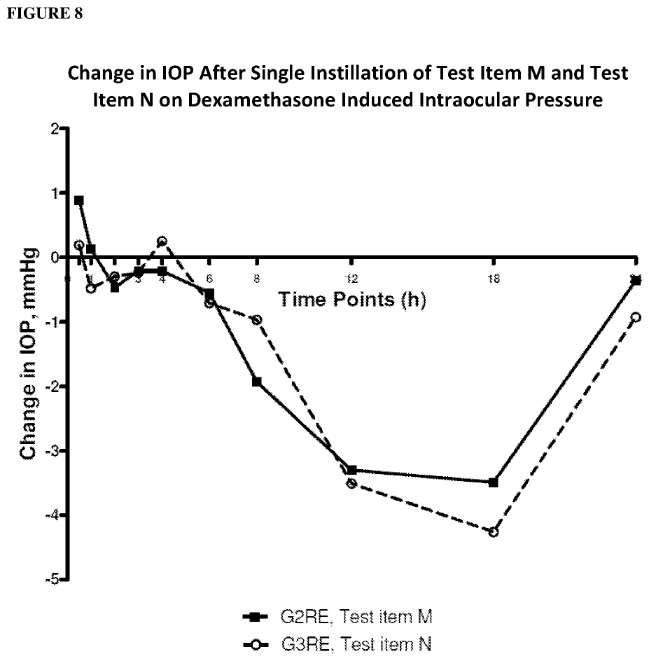

FIG. 8 represents the comparative effect of Test Item M (Xalatan® 0.005%) and Test Item N (P0047-3-3 (1% Cremophor® RH40)) as measured by average change in IOP (mmHg) at each time point 0.5 h to 24-hours. The graphs show no significant difference in average IOP was observed between Test Item M (Xalatan® 0.005%) and Test Item N (P0047-3-3 (1% Cremophor® RH40)) at any of the time points between 0.5 h to 24 hours.

Over the course of the study, rat eyes were observed for miosis. Light was focused by pen tourch on rat eye and pupil contraction was observed. No difference between saline normal control, saline, Test Item M (Xalatan® 0.005%), and Test Item N (P0047-3-3 (1% Cremophor® RH40)) was observed. Further, no conjunctival hyperemia was observed in any of the treatment groups.

### Summary

The intraocular pressure study data described here and provided in FIGS. 1-8 herein demonstrate that the P0047-3-3 (1% Cremophor® RH40) formulation is capable of reducing intraocular pressure in a manner comparable to that of the on-market Xalatan® 0.005% product and is capable of doing so by inclusion of polyoxyl 40 hydrogenated castor oil (Cremophor, e.g., Cremophor RH40) whereby the formulation is stable when stored under typical room temperature conditions.

1. A method for modulating one or more physiological conditions of the eye in a mammalian subject comprising administering a therapeutically effective amount of an ophthalmologically suitable composition comprising about 0.001% to about 0.01% of one or more ophthalmologically suitable latanoprost compounds and about 0.5% to about 2.25% of a polyoxyl castor oil composition, at least about 50% of the polyoxyl castor oil composition being composed of one or more hydrogenated polyoxyl castor oil compounds having 35-50 oxyethylene units, to one or more eyes of the mammalian subject, wherein the composition retains at least about 98% of the initial latanoprost compound content of the composition when the composition is stored at about 22° C. to about 25° C. and about 60% relative humidity for a period of at least about 3 months.

2. The method of claim 1, wherein the one or more ophthalmologically suitable latanoprost compounds is latanoprost.

3. The method of claim 2, wherein the composition comprises about 0.0025% to about 0.0075% latanoprost.

4. The method of claim 1 wherein the steps of the method are performed a sufficient number of times throughout a treatment period to effectively reduce intraocular pressure in a significant number of mammalian subjects in an adequate and well controlled clinical study of mammalian subjects as established by one or more clinical studies or through bioequivalence.

5. The method of claim 4, wherein the mammalian subject is a human patient diagnosed with open-angle glaucoma and the steps of the method are performed a sufficient number of times throughout the treatment period to treat open-angle glaucoma in a significant number of patients in an adequate and well controlled clinical study of mammalian subjects as established by one or more clinical studies or through bioequivalence.

6. The method of claim 1, wherein the method comprises administering one drop of an effective amount of the ophthalmologically suitable composition from a container adapted to administer eye drops.

7. The method of claim 6, wherein the container is subjected to gamma ray sterilization prior to the composition being stored in the container.

8. The method of claim 6, wherein each drop contains about 1.5 μg of one or more latanoprost compounds.

9. The method of claim 5, wherein the method comprises shipping the ophthalmologically suitable composition at temperatures above about 15° C.

10. The method of claim 6, wherein the method comprises administering the composition one or more times to the subject after an initial administration from the container adapted to administer eye drops and maintenance of the composition at a temperature of greater than about 20° C. fora period of at least about 8 weeks.

11. The method of claim 4, wherein the method comprises administering the ophthalmologically suitable composition to the mammalian subject for a period of at least about six months, wherein the mammalian subject experiences an average decrease in intraocular pressure of at least about 15% during the treatment period, at the end of the treatment period, or following the end of the treatment period.

12. The method of claim 4, wherein a significant number of mammalian subjects in a population of mammalian subjects in an adequate and well controlled clinical study exhibit a decrease of IOP of at least about 20% during the treatment period, throughout the treatment period, or following the treatment period.

13. The method of claim 5, wherein the method comprises administering to the human patient an amount of one or more secondary antiglaucoma agents, wherein the amount of the one or more secondary antiglaucoma agents is effective, alone or in combination with the one or more latanoprost compounds of the composition, to significantly reduce one or more symptoms of open-angle glaucoma in a statistically significant number of patients in one or more adequate and well controlled clinical studies as established by such studies or bioequivalence.

14. The method of claim 13, wherein at least one of the one or more secondary antiglaucoma agents is administered to the human patient with the one or more latanoprost compounds as a combination composition.

15. The method of claim 13, wherein at least some of the one or more secondary antiglaucoma agents is administered to the human patient separately from the composition.

16. The method of claim 4, wherein the mammalian subject is a subject diagnosed as suffering from an increased intraocular pressure, and wherein performing the method results in a 0.5-hour post administration to 24-hour post administration percent reduction in intraocular pressure in a statistically significant number of mammalian subjects in one or more adequate and well controlled clinical studies which is at least statistically similar to or detectably greater than that the reduction in intraocular pressure achieved by treating similar subjects for the same period of time with a substantially similar amount of the composition approved by the United States Food and Drug Administration under NDA number 020597 as determined by one or more clinical studies or bioequivalence.

17. The method of claim 16, wherein the method results in a detectably greater reduction in intraocular pressure in a significant number of patients, as determined by bioequivalence or one or more clinical studies, as compared to the composition approved by the United States Food and Drug Administration under NDA number 020597 12-24 hours after administration.

18. The method of claim 17, wherein the method results in a detectably greater reduction in intraocular pressure in a significant number of patients, as determined by bioequivalence or one or more clinical studies, as compared to the composition approved by the United States Food and Drug Administration under NDA number 020597 18-24 hours after administration.

19. The method of claim 4, wherein the composition comprises less than about 0.001% of any polyacrylic acid polymer polyvinyl alcohol, castor oil, boric acid, EDTA, glycerin, propylene glycol, sorbic acid, hydroxypropyl cellulose, hydroxypropyl methylcellulose polyvinylpyrrolidone, tartaric acid, hydroxystearate, macrogolglycerol, aminocaproic acid, cyclodextrin, trehalose, polyethylene glycol sorbitol, mannitol, polyethylene glycol hydroxy stearate, macrogol compounds, propylene glycol, or a combination of any thereof.

20. The method of claim 19, wherein the composition comprises benzalkonium chloride in a concentration of less than about 0.2 mg/mL.
